SEQUENCE LISTING (70 sequences)

SEQ ID NO: 1 - DNA, 10823 bp, Artificial sequence
  Synthetic
gtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcttgcgccgttccacttcgatgcgtcagtgaagcgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgctaatacgactcactatagggagaagcttaatgatacggcgaccaccgacactgctgaggacactctttccctacacgacgctcttccgatctccacgtgcatgctggatccatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattagcccgcctaatgagcgggcttttttttggatccagcatgcacgtggagatcggaagagcggttcagcaggaatgccgagaccgatcctcagcagtgtcgtatgccgtcttctgcttgagatcctatagtgtcacctaaatcgtatgcggccgcccgggccgtcgaccaattctcatgtttgacagcttatcatcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtaggctcgccgcagtcgagcgacggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgaggctttttggcctctgtcgtttcctttctctgtttttgtccgtggaatgaacaatggaagtcctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagac

SEQ ID NO: 2 - DNA, 9905 bp, Artificial sequence
  Synthetic
gtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcttgcgccgttccacttcgatgcgtcagtgaagcgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgctaatacgactcactatagggagaagcttaatgatacggcgaccaccgacactgctgaggacactctttccctacacgacgctcttccgatctccacgtgcatgctggatccagcatgcacgtggagatcggaagagcggttcagcaggaatgccgagaccgatcctcagcagtgtcgtatgccgtcttctgcttgagatcctatagtgtcacctaaatcgtatgcggccgcccgggccgtcgaccaattctcatgtttgacagcttatcatcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtaggctcgccgcagtcgagcgacggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgaggctttttggcctctgtcgtttcctttctctgtttttgtccgtggaatgaacaatggaagtcctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagac

SEQ ID NO: 3 - DNA, 7562 bp, Artificial sequence
  Synthetic
gcggccgcaaggggttcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcacactctttccctacacgacgctcttccgatctcacgtgagatcggaagagcggttcagcaggaatgccgagggatcctctagagtcgacctgcaggcatgcaagcttgagtattctatagtctcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcga

SEQ ID NO: 4 - DNA, 7571 bp, Artificial sequence
  Synthetic
  misc_feature (411)..(418): n is a, c, g, or t
gcggccgcaaggggttcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcacactctttccctacacgacgctcttccgatctcacgtgagatcggaagagcacacgtctgaactccagtcacnnnnnnnnggatcctctagagtcgacctgcaggcatgcaagcttgagtattctatagtctcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcga

SEQ ID NO: 5 - DNA, 1097 bp, Artificial sequence
  Synthetic
aagcttaatgatacggcgaccaccgacactgctgaggacactctttccctacacgacgctcttccgatctccacgtgcatgctggatccatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattagcccgcctaatgagcgggcttttttttggatccagcatgcacgtggagatcggaagagcggttcagcaggaatgccgagaccgatcctcagcagtgtcgtatgccgtcttctgcttgagatct

SEQ ID NO: 6-11 - skipped (6 entries)

SEQ ID NO: 12 - DNA, 58 bp, Artificial sequence
  Synthetic
aatgatacggcgaccaccgagatctacactctttccctacacgacgctcttccgatct

SEQ ID NO: 13 - DNA, 55 bp, Artificial sequence
  Synthetic
caagcagaagacggcatacgagattaagcatggtgactggagttcagacgtgtgc

SEQ ID NO: 14 - DNA, 55 bp, Artificial sequence
  Synthetic
caagcagaagacggcatacgagatgaattgctgtgactggagttcagacgtgtgc

SEQ ID NO: 15 - DNA, 55 bp, Artificial sequence
  Synthetic
caagcagaagacggcatacgagatcctggtaggtgactggagttcagacgtgtgc

SEQ ID NO: 16 - DNA, 55 bp, Artificial sequence
  Synthetic
caagcagaagacggcatacgagataagcaactgtgactggagttcagacgtgtgc

SEQ ID NO: 17 - DNA, 55 bp, Artificial sequence
  Synthetic
caagcagaagacggcatacgagatgtcgagcagtgactggagttcagacgtgtgc

SEQ ID NO: 18 - DNA, 55 bp, Artificial sequence
  Synthetic
caagcagaagacggcatacgagatagatgtgcgtgactggagttcagacgtgtgc

SEQ ID NO: 19 - DNA, 55 bp, Artificial sequence
  Synthetic
caagcagaagacggcatacgagataggctcaagtgactggagttcagacgtgtgc

SEQ ID NO: 20 - DNA, 55 bp, Artificial sequence
  Synthetic
caagcagaagacggcatacgagatctaactgggtgactggagttcagacgtgtgc

SEQ ID NO: 21 - DNA, 35 bp, Artificial sequence
  Synthetic
gctgaggacactctttccctacacgacgctcttcc

SEQ ID NO: 22 - DNA, 38 bp, Artificial sequence
  Synthetic
atcggaagagcgtcgtgtagggaaagagtgtcctcagc

SEQ ID NO: 23 - DNA, 44 bp, Artificial sequence
  Synthetic
atcggaagagcggttcagcaggaatgccgagaccgatcctcagc

SEQ ID NO: 24 - DNA, 41 bp, Artificial sequence
  Synthetic
gctgaggatcggtctcggcattcctgctgaaccgctcttcc

SEQ ID NO: 25 - DNA, 13 bp, Artificial sequence
  Synthetic
gatctcatgctta

SEQ ID NO: 26 - DNA, 10 bp, Artificial sequence
  Synthetic
taagcatgag

SEQ ID NO: 27 - DNA, 10 bp, Artificial sequence
  Synthetic
taagcatgag

SEQ ID NO: 28 - DNA, 13 bp, Artificial sequence
  Synthetic
gatctcatgctta

SEQ ID NO: 29 - DNA, 47 bp, Artificial sequence
  Synthetic
gctgaggacactctttccctacacgacgtcttccgatctcatgctta

SEQ ID NO: 30 - DNA, 54 bp, Artificial sequence
  Synthetic
taagcatgagatcggaagagcggttcagcaggaatgccgagaccgatcctcagc

SEQ ID NO: 31 - DNA, 48 bp, Artificial sequence
  Synthetic
taagcatgagatcggaagagcgtcgtgtagggaaagagtgtcctcagc

SEQ ID NO: 32 - DNA, 54 bp, Artificial sequence
  Synthetic
gctgaggatcggtctcggcattcctgctgaaccgctcttccgatctcatgctta

SEQ ID NO: 33 - DNA, 13 bp, Artificial sequence
  Synthetic
gatctcatgctta

SEQ ID NO: 34 - DNA, 10 bp, Artificial sequence
  Synthetic
taagcatgag

SEQ ID NO: 35 - DNA, 13 bp, Artificial sequence
  Synthetic
gatctagcaattc

SEQ ID NO: 36 - DNA, 10 bp, Artificial sequence
  Synthetic
gaattgctag

SEQ ID NO: 37 - DNA, 13 bp, Artificial sequence
  Synthetic
gatctctaccagg

SEQ ID NO: 38 - DNA, 10 bp, Artificial sequence
  Synthetic
cctggtagag

SEQ ID NO: 39 - DNA, 13 bp, Artificial sequence
  Synthetic
gatctagttgctt

SEQ ID NO: 40 - DNA, 10 bp, Artificial sequence
  Synthetic
aagcaactag

SEQ ID NO: 41 - DNA, 13 bp, Artificial sequence
  Synthetic
gatcttgctcgac

SEQ ID NO: 42 - DNA, 10 bp, Artificial sequence
  Synthetic
gtcgagcaag

SEQ ID NO: 43 - DNA, 13 bp, Artificial sequence
  Synthetic
gatctgcacatct

SEQ ID NO: 44 - DNA, 10 bp, Artificial sequence
  Synthetic
agatgtgcag

SEQ ID NO: 45 - DNA, 13 bp, Artificial sequence
  Synthetic
gatctttgagcct

SEQ ID NO: 46 - DNA, 10 bp, Artificial sequence
  Synthetic
aggctcaaag

SEQ ID NO: 47 - DNA, 13 bp, Artificial sequence
  Synthetic
gatctccagttag

SEQ ID NO: 48 - DNA, 10 bp, Artificial sequence
  Synthetic
ctaactggag

SEQ ID NO: 49 - DNA, 20 bp, Artificial sequence
  Synthetic
  misc_feature (1)..(1): Immobilized on flow cell surface 54-PSTTTTTTTTTT-( diol)3
aatgatacggcgaccaccga

SEQ ID NO: 50 - DNA, 31 bp, Artificial sequence
  Synthetic
  misc_feature (1)..(1): Immobilized on flow cell surface 54-PS
ttttttttttcaagcagaagacggcatacga

SEQ ID NO: 51 - DNA, 39 bp, Artificial sequence
  Synthetic
  misc_feature (1)..(1): Immobilized on flow cell surface 54-PS
  misc_feature (33)..(33): N = 2-deoxyuridine
ttttttttttaatgatacggcgaccaccgaganctacac

SEQ ID NO: 52 - DNA, 34 bp, Artificial sequence
  Synthetic
  misc_feature (1)..(1): Immobilized on flow cell surface 54-PS
  misc_feature (32)..(32): N = 8-oxoguanine
ttttttttttcaagcagaagacggcatacganat

SEQ ID NO: 53 - DNA, 33 bp, Artificial sequence
  Synthetic
acactctttccctacacgacgctcttccgatct

SEQ ID NO: 54 - DNA, 37 bp, Artificial sequence
  Synthetic
cggtctcggcattcctgctgaaccgctcttccgatct

SEQ ID NO: 55 - DNA, 50 bp, Artificial sequence
  Synthetic
acactctttccctacacgacgctcttccgatctgtacccggggatcccac

SEQ ID NO: 56 - DNA, 53 bp, Artificial sequence
  Synthetic
ctcggcattcctgctgaaccgctcttccgatcttcgactctagaggatcccac

SEQ ID NO: 57 - DNA, 25 bp, Artificial sequence
  Synthetic
ataaaaaactcagaacggcttcgta

SEQ ID NO: 58 - DNA, 13 bp, Artificial sequence
  Synthetic
  misc_feature (1)..(1): 5′ Phosphorylated
gatctcatgctta

SEQ ID NO: 59 - DNA, 10 bp, Artificial sequence
  Synthetic
  misc_feature (1)..(1): 5′ Phosphorylated
taagcatgag

SEQ ID NO: 60 - DNA, 13 bp, Artificial sequence
  Synthetic
  misc_feature (1)..(1): 5′ Phosphorylated
gatctcatgctta

SEQ ID NO: 61 - DNA, 10 bp, Artificial sequence
  Synthetic
  misc_feature (1)..(1): 5′ Phosphorylated
taagcatgag

SEQ ID NO: 62 - DNA, 13 bp, Artificial sequence
  Synthetic
  misc_feature (1)..(1): 5′ Phosphorylated
gatctcatgctta

SEQ ID NO: 63 - DNA, 10 bp, Artificial sequence
  Synthetic
taagcatgag

SEQ ID NO: 64 - DNA, 10 bp, Artificial sequence
  Synthetic
taagcatgag

SEQ ID NO: 65 - DNA, 14 bp, Artificial sequence
  Synthetic
  misc_feature (1)..(1): 5′ Phosphorylated
gatctcatgcttat

SEQ ID NO: 66 - DNA, 11 bp, Artificial sequence
  Synthetic
  misc_feature (2)..(2): 5′ Phosphorylated
ataagcatgag

SEQ ID NO: 67 - DNA, 11 bp, Artificial sequence
  Synthetic
  misc_feature (1)..(1): 5′ Phosphorylated
ataagcatgag

SEQ ID NO: 68 - DNA, 14 bp, Artificial sequence
  Synthetic
  misc_feature (1)..(1): 5′ Phosphorylated
gatctcatgcttat

SEQ ID NO: 69 - DNA, 9902 bp, Artificial sequence
  Synthetic
gtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcttgcgccgttccacttcgatgcgtcagtgaagcgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgctaatacgactcactatagggagaagcttaatgatacggcgaccaccgacactgctgaggacactctttccctacacgacgctcttccgatctccacgtgcatgctggatccagcatgcacgtggagatcggaagagcacacgtctgaactccagtcacatcctcagcagtgtcgtatgccgtcttctgcttgagatcctatagtgtcacctaaatcgtatgcggccgcccgggccgtcgaccaattctcatgtttgacagcttatcatcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtaggctcgccgcagtcgagcgacggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgaggctttttggcctctgtcgtttcctttctctgtttttgtccgtggaatgaacaatggaagtcctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagac

SEQ ID NO: 70 - DNA, 9863 bp, Artificial sequence
  Synthetic
gtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaggagcgcccaatacgcaaaccgcctctgtttaaacgttggccgattcattaatgcagcttgcgccgttccacttcgatgcgtcagtgaagcgacatgaggcgcgcccgtattcagtgtcgctgatttgtattgtctgaagttgtttttacgttaagttgaagcagatcaattaatacgatacctgcgtcataattgattatttgacgtggtttgatggcctccacgcacgttgtgatatgtagaagataatcattatcactttacgggtcctttccggtgatccgacaggttacggggcggcgacctcgcgggttttcgctatttatgaaaattttccggtttaaggcgtttccgttcttcttcgtcataacttaatgaatttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgctaatacgactcactatagggagaagcttgctgaggacactctttccctacacgacgctcttccgatcttcatccacgtgcatgctggatcctacctcattcgtgatacagatcggaagagcggttcagcaggaatgccgagaccgatcctcagcgatcctatagtgtcacctaaatcgtatgcggccgcccgggccgtcgaccaattctcatgtttgacagcttatcatcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtaggctcgccgcagtcgagcgacggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcctcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgagttggttatacaattgaacaaattaatcatatgcgtgatgtgtttggtacgcgattgcgacgtgctgaagacgtatttccaccggtgatcggggttgctgctcataaaggtggcgtttacaaaacctcagtttctgttcatcttgctcaggatctggctctgaaggggctacgagttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcacctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcctcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgaggctttttggcctctgtcgtttcctttctctgtttttgtccgtggaatgaacaatggaagtcctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagac

(19) United States
(12) Patent Application Publication
(10) Pub. No.: US 2014/0228223 A1
(43) Pub. Date: Aug. 14, 2014

(54) HIGH THROUGHPUT PAIRED-END SEQUENCING OF LARGE-INSERT CLONE LIBRARIES

(71) Applicants: Andreas Gnirke, Wellesley, MA (US); Robert Nicol, Cambridge, MA (US); Louise Williams, Reading, MA (US); Maura T. Costello, Malden, MA (US); Scott Steelman, Woburn, MA (US)

(72) Inventors: Andreas Gnirke, Wellesley, MA (US); Robert Nicol, Cambridge, MA (US); Louise Williams, Reading, MA (US); Maura T. Costello, Malden, MA (US); Scott Steelman, Woburn, MA (US)

(21) Appl. No.: 13/697,108

(22) PCT Filed: May 10, 2011
(86) PCT No.: PCT/US11/35940

§ 371 Date: Mar. 25, 2014

Related U.S. Application Data
(60) Provisional application No. 61/333,127, filed on May 10, 2010.
(60) Provisional application No. 61/426,735, filed on Dec. 23, 2010.

(51) Int. Cl. C12Q 1/68

(52) U.S. Cl. CPC C12Q 1/6874
USPC 506/2; 435/320.1; 506/26; 506/14

(57) ABSTRACT

The present invention is related to genomic nucleotide sequencing. In particular, the invention describes a paired end sequencing method that improves the yield of long-distance genomic read pairs by constructing long-insert clone libraries (i.e., for example, a fosIll library or a fosCN library) and converting the long-insert clone library using inverse polymerase chain reaction amplification or shearing and recircularization of shortened fragments into a library of co-ligated clone-insert ends. The resultant jumping libraries are compatible with massively parallel sequencing techniques. The compositions and methods disclosed herein contemplate sequencing complex genomes as well as detecting chromosomal structural rearrangements.

### STATEMENT OF GOVERNMENT SUPPORT

This invention was made with government support under HG03067-05 awarded by the National Human Genome Research Institute. The government has certain rights in the invention.

### FIELD OF THE INVENTION

The present invention is related to genomic nucleotide sequencing. In particular, the invention describes a paired end sequencing methods that yields unique read pairs by co-localizing both both ends of a genomic DNA fragment that has been inserted into a cloning vector and propagated in a microbial host on a single polymerase chain reaction product. The methods may use customized cloning vector that contains primer pairs that are compatible with massively parallel sequencing techniques. The compositions and methods disclosed herein contemplate sequencing complex genomes as well as detecting chromosomal structural rearrangements.

### BACKGROUND

Recent advances in sequencing technology have rapidly driven down the cost of DNA sequence data and yield an unrivalled resource of genetic information. Individual genomes can be characterized, while genetic variation may be studied in populations and disease. Until recently, the scope of sequencing projects was limited by the cost and throughput of Sanger sequencing. The raw data for the three billion base (3 gigabase (Gb)) human genome sequence was generated over several years for ˜$300 million using several hundred capillary sequencers. International Human Genome Sequencing Consortium, “Finishing the euchromatic sequence of the human genome” Nature 431:931-945 (2004). More recently, an individual human genome sequence has been determined for ˜$10 million by capillary sequencing. Levy et al., “The diploid genome sequence of an individual human” PLoS Biol. 5:e254 (2007). Several new approaches at varying stages of development aim to increase sequencing throughput and reduce cost. Margulies et al., “Genome sequencing in microfabricated high-density picolitre reactors” Nature 437:376-380 (2005); Shendure et al., “Accurate multiplex polony sequencing of an evolved bacterial genome” Science 309:1728-1732 (2005); Harris et al., “Single-molecule DNA sequencing of a viral genome” Science 320:106-109 (2008); and Lundquist et al., “Parallel confocal detection of single molecules in real time” Opt. Lett. 33:1026-1028 (2008). These techniques increase parallelization markedly by imaging many DNA molecules simultaneously. One instrument run produces typically thousands or millions of sequences that are shorter than capillary reads. Another human genome sequence was recently determined using one of these approaches. Wheeler et al., “The complete genome of an individual by massively parallel DNA sequencing” Nature 452:872-876 (2008). Moreover, an international consortium is currently in the process of determining the genome sequence of at least a thousand different human individuals (1000genomes.org/page.php?page=home). These human genome sequences are typically based on the pre-existing human reference sequence and are not assembled de novo (i.e., without prior knowledge of the reference sequence)

However, further improvements are necessary to improve the efficiency of these massively parallel sequencing systems to enable routine sequencing and assembly of complex genomes de novo (i.e., without a pre-existing reference sequence). Essentially all methods for assembling genomes de novo require pairs of sequencing reads that have an a priori defined orientation and spacing in the underlying genome. Long-distance (i.e., for example 30-45 kb) read pairs are particularly important to provide long-range contiguity of genome assemblies. Without such long-distance read pairs, genome assemblies remain highly fragmented. Approaches that improve the yield of long-distance read pairs by massively-parallel sequencing and thus the quality of genome assemblies would greatly facilitate biological and medical research.

The advent of next generation sequencing technologies has vastly increased the number of bases sequenced each year while drastically reducing the cost. Such technologies as the Illumina GAIIx platform enable efficient paired end sequencing of short fragments from 150-500 bp. While this size of insert reads have shown great utility for a variety of applications, de novo genome assembly needs to generate data with larger inserts (i.e., for example, ˜40 kb).

### SUMMARY

The present invention is related to genomic nucleotide sequencing. In particular, the invention describes a paired end sequencing method that improves the yield of unique read pairs that are far (i.e., for example, 1-1000 kb) apart in the genome. The method may use an inverse polymerase amplification, or shearing in combination with re-circularization, to convert a large-insert clone library (i.e., for example, a fosmid library) representing the genome to a plurality of linear amplification products (read pair jumping library) that are compatible with massively parallel sequencing techniques. The compositions and methods disclosed herein contemplate sequencing complex genomes as well as detecting chromosomal structural rearrangements.

In one embodiment, the present invention contemplates a composition comprising a library of large-insert microbial clones. In one embodiment, the large-insert clones are compatible with whole-genome shotgun sequencing. In one embodiment, the library comprises a fosmid library. In one embodiment, the library comprises at least one nucleic acid sequence comprising a universal forward primer recognition site and a universal reverse primer recognition site, wherein the forward and the reverse primer sites are separated by approximately 1 kb-1000 kb, but more preferably between approximately 30-45 kb. In one embodiment, the primer sites are separated by a cloned genome fragment. Although it is not necessary to understand the mechanism of an invention, it is believed that the large-insert clone library supports paired end sequencing of ˜40 kb read-pairs, thereby providing long-range contiguity of sequence assemblies. It is further believed that a fosmid library approach for generating read pairs spanning ˜40 kb can support de novo next-generation sequencing of complex genomes, as well as the detection of chromosomal structural rearrangements such as translocations or inversions.

In one embodiment, the present invention contemplates a composition comprising a first nucleic acid sequence comprising a cloning site, wherein the cloning site is flanked by a universal primer sequence pair and an endonuclease site pair. In one embodiment, the cloning site comprises at least two restriction enzyme recognition site clusters. In one embodiment, the at least two restriction enzyme recognition site clusters are identical. In one embodiment, the restriction enzyme recognition site clusters comprise polylinkers. In one embodiment, the composition further comprises a second nucleic acid sequence ligated to the polylinkers, wherein the second nucleic acid sequence is inserted between the polylinkers. In one embodiment, the second nucleic acid sequence ranges between approximately 10-200 kb. In one embodiment, the second nucleic acid sequence is approximately 40 kb. In one embodiment, the first nucleic acid sequence comprises an F-plasmid derived vector sequence. In one embodiment, the universal primer sequence pair comprises a bridge amplification primer pair (i.e., for example, Illumina). In one embodiment, the universal primer sequence pair comprises an emulsion amplification primer pair (i.e., for example, SOLiD). In one embodiment, the endonuclease site pair comprises a Nb/BbvC1 nicking endonuclease site pair.

In one embodiment, the present invention contemplates a composition comprising a circular first nucleic acid sequence comprising a cloning site, wherein the cloning site is flanked by a sequencing primer pair binding site. In one embodiment, the cloning site is flanked by a first genomic sequence and a second genomic sequence. In one embodiment, the cloning site is further flanked by a polymerase chain reaction (PCR) enrichment primer binding site. In one embodiment, the first genomic sequence is approximately 100 base pairs. In one embodiment, the second genomic sequence is approximately 100 base pairs. In one embodiment, the first nucleic acid comprises a vector sequence. In one embodiment, the vector sequence comprises a barcode adapter sequence. In one embodiment, the first nucleic acid sequence comprises a pFosCN-1 vector sequence. In one embodiment, the pFosCN-1 vector sequence comprises a pEpiFos-5 vector sequence. In one embodiment, the first nucleic acid sequence comprises a pFosCN-2 vector sequence. In one embodiment, the first nucleic acid sequence comprises a barcode. In one embodiment, the barcode is genome specific. In one embodiment, the genome specific barcode is a species specific barcode. In one embodiment, the composition further comprises a second nucleic acid sequence ranging between approximately 2-200 kb. In one embodiment, the second nucleic acid sequence is approximately 40 kb. In one embodiment, the second nucleic acid sequence comprises genomic deoxyribonucleic acid. In one embodiment, the genomic deoxyribonucleic acid comprises mammalian deoxyribonucleic acid. In one embodiment, the mammalian deoxyribonucleic acid is selected from the group consisting of human deoxyribonucleic acid, mouse deoxyribonucleic acid, or Three-spine Stickleback deoxyribonucleic acid. In one embodiment, the composition comprises a ShaRc fosmid fragment.

In one embodiment, the present invention contemplates a composition comprising a plurality of microbial clones, wherein each of the clones comprise a first nucleic acid sequence comprising a cloning site, wherein said cloning site comprises at least two restriction enzyme recognition site clusters and is flanked by a universal primer sequence pair and a nicking endonuclease site pair. In one embodiment, the composition further comprises a second nucleic acid sequence of approximately 10-1000 kb, but more preferably between 0.30-45 kb, in length inserted into the cloning site. In one embodiment, the recognition site clusters comprise polylinkers. In one embodiment, the recognition site cluster pairs are identical. In one embodiment, the microbial clone comprises an E. coli clone. In one embodiment, each of the second nucleic acid sequences are derived from the same genome, thereby creating a genomic library. In one embodiment, the first nucleic acid sequence comprises an F-plasmid-derived vector sequence. In one embodiment, the universal primer sequence pair comprises a bridge amplification primer sequence. (i.e., for example, Illumina). In one embodiment, the universal primer sequence pair comprises an emulsion amplification primer sequence (i.e., for example, SOLiD. In one embodiment, the endonuclease site pair comprises a Nb/BbvC1 endonuclease site pair.

In one embodiment, the present invention contemplates a composition comprising a plurality of microbial clones, wherein each of the clones comprise a circular first nucleic acid sequence comprising a cloning site, wherein said cloning site is flanked by a sequencing primer pair binding site. In one embodiment, the cloning site is further flanked by a polymerase chain reaction (PCR) primer binding site. In one embodiment, the composition further comprises a second nucleic acid sequence of approximately 2-1000 kb, but more preferably between 30-45 kb, in length inserted into the cloning site. In one embodiment, the microbial clone comprises an E. coli clone. In one embodiment, the second nucleic acid sequence is a deoxyribonucleic acid sequence. In one embodiment, the second nucleic acid sequence is a genomic nucleic acid sequence. In one embodiment, the second nucleic acid sequence in each clone is derived from the same deoxyribnucleic acid sequence sample, thereby creating a deoxyribonucleic acid sample library. In one embodiment, the second nucleic acid sequence in each clone is derived from the same genome, thereby creating a genomic library. In one embodiment, the first nucleic acid sequence comprises a vector sequence. In one embodiment, the vector sequence comprises a barcode adapter sequence. In one embodiment, the vector sequence comprises a fosmid vector sequence. In one embodiment, the fosmid vector sequence comprises a pFosCN-1 vector sequence. In one embodiment, the pFos-CN-1 vector sequence comprises a pEpiFos-5 vector sequence. In one embodiment, the fosmid vector sequence comprises a pFosCN-2 vector sequence. In one embodiment, the first nucleic acid sequence comprises a barcode. In one embodiment, the barcode is deoxyribonucleic acid sample specific. In one embodiment, the barcode is genome specific.

In one embodiment, the present invention contemplates a method comprising: a) providing; i) an F-plasmid-derived cloning vector comprising a cloning site, wherein the cloning site comprises at least two polylinkers and is flanked by a universal primer sequence pair and a a nicking endonuclease site pair; ii) a genomic nucleic acid insert ranging between approximately 10-1000 kb, but more preferably between approximately 30-45 kb; and iii) a microbe capable of being transfected by the F-plasmid-derived vector; b) incorporating the genomic nucleic acid insert into the cloning site of the F-plasmid-derived vector, thereby creating a fosmid; c) transfecting the fosmid into the microbe, thereby forming a fosmid clone library; d) amplifying the fosmid library in vivo by growing the library of transfected microbes in a suitable culture medium, e) extracting and purifying the circular fosmids; e) cleaving the cloned inserts to create a first portion and a second portion, wherein the first portion is less than the second portion, and the first portion remains attached to the F-plasmid-derived cloning vector, and the second portion is released from the cloning vector; f) recircularizing the cloning vector by co-ligating the terminal ends of the first portion; g) amplifying the first portion by inverse polymerase chain reaction using the universal primer pair, thereby creating a plurality of linear amplicons configured for at least one next-generation sequencing platform. In one embodiment, the first portion is <1 kb. In one embodiment, the second portion is >39 kb. In one embodiment, the fosmid cloning vector comprises a modified pFOS1 vector. In another embodiment, the fosmid cloning vector comprise other suitably modified plasmid, cosmid, fosmid or Bacterial Artificial Chromosome (BAC) cloning vector. In one embodiment, the method further comprises nicking the endonuclease site under conditions such that the nick can be moved (“translated”) by nick translation several hundred base pairs into the cloned insert. In one embodiment, the endonuclease site comprises a Nb/BbvC1 endonuclease site. In one embodiment, double-strand breaks at the translated nicks are generated by S1 nuclease. In one embodiment, the fosmid cloning library comprises a fosIll cloning library. In one embodiment, the universal primer sequence comprises a bridge amplification primer sequence. In one embodiment, the at least one next-generation sequencing platform comprises a bridge amplification sequencing platform. In one embodiment, the universal primer sequence comprises an emulsion amplification primer sequence. In one embodiment, the at least one next-generation platform comprises an emulsion amplification platform.

In one embodiment, the present invention contemplates a method comprising: a) providing; i) a vector comprising a cloning site, wherein the cloning site is flanked by an Illumina adapter sequence pair binding site and a polymerase chain reaction (PCR) enrichment primer binding site; ii) a genomic nucleic acid insert ranging between approximately 2-1000 kb, but more preferably between approximately 30-45 kb; iii) a microbe capable of being transfected by the vector; and vi) a primer specific for the PCR enrichment primer binding site; b) incorporating the genomic nucleic acid insert into the cloning site of the vector, thereby creating a plurality of clones; c) transfecting the plurality of clones into the microbe, thereby forming a clone genomic library; d) amplifying the genomic library in vivo by growing the library of transfected microbes in a suitable culture medium, e) extracting and purifying the amplified clone deoxyribonucleic acid; e) hydroshearing the amplified clone deoxyribonucleic acid to create a first portion and a second portion, wherein the first portion comprises the vector flanked by a first genomic nucleic acid sequence and second genomic nucleic acid sequence and the second portion is released from the cloning vector; f) re-circularizing the first portion by co-ligating the terminal ends of the first genomic nucleic acid sequence and second genomic nucleic acid sequence, thereby creating a circularized first portion; g) amplifying the circularized first portion with the enrichment primer, thereby creating a plurality of linear amplicons comprising a mate read pair configured for at least one next-generation sequencing platform. In one embodiment, the vector comprises a barcode adapter sequence. In one embodiment, the mate read pair comprises a read one and a read two. In one embodiment, the read one is approximately 100 base pairs. In one embodiment, the read two is approximately 100 base pairs. In one embodiment, the first portion ranges between approximately 8-10 kb. In one embodiment, the first portion comprises the vector flanked by a genomic insert ranging between approximately 250-1250 base pairs. In one embodiment, the second portion is approximately 39 kb. In one embodiment, the vector comprises a fosmid vector. In one embodiment, the fosmid vector comprises a modified pFosCN-1 vector. In one embodiment, the modified pFosCN-1 vector comprises a pEpiFos-5 vector sequence. In one embodiment, the fosmid cloning library comprises a fosIll cloning library. In one embodiment, the circularized first portion is a ShaRc fosmid fragment.

In one embodiment, the present invention contemplates a method comprising: a) providing; i) a ShaRc fosmid fragment library, wherein the library comprises short read genomic inserts and a fosmid plasmid derived cloning vector sequence; ii) a next-generation sequencing platform; and iii) a microprocessor comprising a genome assembly algorithm; b) sequencing the short read genomic inserts with the next-generation sequencing platform; and c) assembling the short read genomic inserts into a complete genome using the genome assembly algorithm. In one embodiment, the short read genomic inserts are approximately 100 base pairs. In one embodiment, the genome assembly algorithm comprises a step for trimming the fosmid plasmid derived cloning vector sequence from the short read genomic inserts. In one embodiment, the complete genome comprises a microbial genome. In one embodiment, the complete genome comprises a mammalian genome. In one embodiment, the ShaRc fosmid fragment library is a pooled ShaRc fosmid fragment library. In one embodiment, the pooled ShaRc fosmid fragment library comprises a first ShaRc fragment library and a second ShaRc fragment library. In one embodiment, the first and second ShaRc fragment library are derived from the same genome. In one embodiment, the short read genomic inserts comprise a barcode nucleic acid sequence. In one embodiment, the barcode nucleic acid designates a specific genome. In one embodiment, the first and second ShaRc fragment library are derived from different genomes.

In one embodiment, the present invention contemplates a kit comprising: a) a first container comprising a fosmid cloning vector comprising a cloning site wherein said cloning site is flanked by a universal primer sequence and a nicking endonuclease sites. b) a second container comprising an endonuclease capable of nicking the endonuclease site; and c) a third container comprising enzymes and reagents for performing nick translation; and d) a fourth container comprising enzymes and reagents for performing an inverse polymerase chain reaction. In one embodiment, the kit further comprises instructions for incorporating a genomic nucleic acid sequence into the fosmid cloning vector to create a fosmid library. In one embodiment, the fosmid library is a fosmid jumping library. In one embodiment, the kit further comprises instructions for using the fosmid library with next-generation sequencing platforms.

In one embodiment, the present invention contemplates a kit comprising: a) a first container comprising a vector comprising a cloning site wherein said cloning site is flanked by a sequencing primer pair binding site and a polymerase chain reaction (PCR) enrichment primer binding site; b) a second container comprising a deoxyribonucleic acid ligase capable of ligating genomic deoxyribonucleic acid; and c) a third container comprising a plasmid-safe deoxyribonucleic acid exonuclease; and d) a fourth container comprising a primer specific for the PCR enrichment binding site and enzymes and reagents for performing polymerase chain reaction. In one embodiment, the kit further comprises instructions for incorporating a genomic nucleic acid sequence into the vector to create a sub-clone library. In one embodiment, the subclone library is a jumping sub-clone library. In one embodiment, the kit further comprises instructions for using the sub-clone library with next-generation sequencing platforms. In one embodiment, the kit further comprises instructions for using the sub-clone library sequencing data to reconstruct a genome using a genomic assembly program. In one embodiment, the vector comprises a barcode adapter sequence. In one embodiment, the vector is a fosmid vector.

### DEFINITIONS

The term “clone library”, as used herein, refers to any population of organisms, each of which carries a DNA molecule inserted into a cloning vector, or alternatively, to a collection of all of the cloned vector molecules representing a specific genome.

The term “vector”, as used herein refers to any plasmid or bacteriophage that has been used to infect a microorganisms, comprising at least one nucleotide sequence of interest that is preserved as an insert. For example, the vector may include, but is not limited to, a fosmid vector. The methods described herein are not limited to any particular type of vector and may be successfully practiced with any of a number of vector and/or plasmid types including but not limited to fosmids, cosmids, plasmids, Bacterial Artificial Chromosomes (BACs), P1-derived artificial chromosome (PACs), and any circular plasmids or circular chromosome that can be propagated in eukaryotic microbes including but not limited to circular yeast artificial chromosomes (e.g., circular YACs). A “vector” as used herein, may also refer to a polynucleotide sequence comprising an integrated gene sequence that is capable of expression. For example, a gene sequence desired for replication may be inserted into copies of a vector containing genes that make cells resistant to particular antibiotics, for inserting a multiple cloning site (MCS), and/or a polylinker site. An MCS comprises a short region containing several commonly used restriction sites allowing the easy insertion of DNA fragments at this location. Vectors may be inserted into a microorganism (i.e., for example, a bacteria including, but not limited to, E. coli) by transformation.

A “plasmid” as used herein, refers to any DNA molecule that is separate from, and can replicate independently of, the chromosomal DNA. Plasmid DNA may be double stranded and in many cases, spontaneously circularizes. Plasmids usually occur naturally in bacteria, but are sometimes found in eukaryotic organisms (e.g., a 2-micrometer-ring in Saccharomyces cerevisiae). Plasmid size may vary from 1 to over 1,000 kilobase pairs (kbp).

The term “fosmid” as used herein, refers to any plasmid or vector comprising a Fertility, or F-plasmid-derived sequences.

The term “stuffer sequence” as used herein, refers to any ancillary nucleic acid sequence that is added to a vector to modify and/or improve its biological characteristics. For example, a stuffer sequence may include, but is not limited to, a kanamycin sequence, primer sequences, and/or sticky end sequences.

The term “barcode adapter sequence” as used herein, refers to any ancillary nucleic acid sequence that is added to an insert nucleic acid sequence comprising at least one terminal overhang to which a barcode sequence may hybridize.

The term “library”, as used herein refers to a clone library, or alternatively, a library of genome-derived sequences carrying vector sequences. The library may also have sequences allowing amplification of the “library” by the polymerase chain reaction or other in vitro amplification methods well known to those skilled in the art. The library may also have sequences that are compatible with next-generation high throughput sequencers including but not limited to Illumina adapter pair sequences. For example, a “ShaRc fosmid fragment library” refers to a collection of fosmid vector sequences comprising genomic nucleic acid sequences that has been created by the hydroshearing and recircularization method described herein.

The term “short read” as used herein refers to any nucleic acid sequence of ranging between approximately 25-500 base pairs, but preferably ranging between 50-300 base pairs, but even more preferably ranging between approximately 75-150 base pairs, but most preferably approximately 100 base pairs that is compatible with a high throughput sequencer.

The term “next-generation sequencing platform” as used herein, refers to any nucleic acid sequencing device that utilizes massively parallel technology. For example, such a platform may include, but is not limited to, Illumina sequencing platforms.

The term “high throughput sequencer adapter pair” refers to a specific nucleic acid pair that provides compatibility with a massively parallel sequencing platform (i.e., for example, Illumina sequencer adapter pairs).

The term “microprocessor” as used herein refers to any integrated circuit (i.e., a single or multiple circuits) that is capable of executing an algorithm. A website is contemplated herein as encompassed by the term ‘microprocessor’. For example, the micropressor may be configured to execute a genome assembly algorithm (i.e., for example, ALLPATHS-LG). A microprocessor may be integrated into a computer that also contains other microprocessors for a variety of purposes.

The term “genome assembly algorithm” as used herein, refers to any method capable of aligning short reads with reference sequences under conditions that a complete sequence of the genome may be determined.

The term “genome” as used herein, refers to the entirety of an organism's hereditary information that is encoded in its primary DNA sequence. The genome includes both the genes and the non-coding sequences. For example, the genome may represent a microbial genome or a mammalian genome.

The term “barcode” as used herein, refers to any unique, non-naturally occurring, nucleic acid sequence that may be used to identify the originating genome of a nucleic acid fragment.

The term “hydroshearing” as used herein, refers to any method or device capable of processing fosmid genomic library nucleic acid sequences into fragments ranging in size between approximately 2-100 kb, more preferably ranging between approximately 4-50 kb, but more preferably ranging between 6-25 kb, but most preferably ranging between approximately 8-10 kb.

The term “coverage” as used herein, refers to an average number of reads representing a given nucleotide in the reconstructed sequence. It can be calculated from the length of the original genome (G), the number of reads(N), and the average read length(L) as NL/G. For example, a hypothetical genome with 2,000 base pairs reconstructed from 8 reads with an average length of 500 nucleotides will have 2× redundancy. This parameter also enables one to estimate other quantities, such as the percentage of the genome covered by reads (the coverage). A high coverage in shotgun sequencing is desired because it can overcome errors in base calling and assembly. The subject of DNA sequencing theory addresses the relationships of such quantities. Alternatively, the term “coverage” may refer to the average number of genome fragments present in a library covering a given nucleotide in the underlying genome.

The term “S1 nuclease” as used herein, refers to any endonuclease that is active against single-stranded DNA and RNA molecules. Usually, an S1 nuclease demonstrates a preference for DNA molecules, wherein the reaction products comprise oligonucleotides or single nucleotides with 5′ phosphoryl groups. Usually, the endonuclease acts upon a single-stranded substrate but can also occasionally introduce single-stranded breaks in double-stranded DNA or RNA, or DNA-RNA hybrids. For example, the endonuclease may be used to remove single stranded tails from a DNA molecule to create blunt ended molecules and/or opening hairpin loops.

The term “nick” as used herein, refers to any discontinuity in a double stranded DNA molecule where there is no phosphodiester bond between adjacent nucleotides of one strand, typically through the action of a nuclease enzyme.

The term “chain termination” as used herein, refers to any chemical reaction leading to the destruction of a reactive intermediate in a chain propagation step in the course of a polymerization, effectively bringing it to a halt. For example, chain termination may be used in the sequencing of nucleic acid polymers.

The term “bridge amplification” as used herein refers to any polymerase chain reaction that allows the generation of in situ copies of a specific DNA molecule on an oligo-decorated solid support. For example, bridge amplification is performed to produce DNA molecules that are compatible with an Illumina sequencing techniques.

The term “DNA sequencing” as used herein, refers to any methods for determining the order of the nucleotide bases—adenine, guanine, cytosine, and thymine—in a molecule of DNA.

The term “derived from” as used herein, refers to the source of a compound or sequence. In one respect, a compound or sequence may be derived from an organism or particular species. In another respect, a compound or sequence may be derived from a larger complex or sequence.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and is, in a preferred embodiment, free of other genomic nucleic acid).

The term “portion” when used in reference to a nucleotide sequence refers to fragments of that nucleotide sequence. The fragments may range in size from 5 nucleotide residues to the entire nucleotide sequence minus one nucleic acid residue.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” is complementary to the sequence “G-T-C-A.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which is partially complementary, i.e., “substantially homologous,” to a nucleic acid sequence is one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

An oligonucleotide sequence which is a “homolog” is defined herein as an oligonucleotide sequence which exhibits greater than or equal to 50% identity to a sequence, when sequences having a length of 100 bp or larger are compared.

Low stringency conditions comprise conditions equivalent to binding or hybridization at 42° C. in a solution consisting of 5×SSPE (43.8 g/1 NaCl, 6.9 g/l NaH₂PO₄.H₂O and 1.85 g/l EDTA, pH adjusted to 7.4 with NaOH), 0.1% SDS, 5×Denhardt's reagent {50×Denhardt's contains per 500 ml: 5 g Ficoll (Type 400, Pharmacia), 5 g BSA (Fraction V; Sigma)} and 100 μg/ml denatured salmon sperm DNA followed by washing in a solution comprising 5×SSPE, 0.1% SDS at 42° C. when a probe of about 500 nucleotides in length. is employed. Numerous equivalent conditions may also be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol), as well as components of the hybridization solution may be varied to generate conditions of low stringency hybridization different from, but equivalent to, the above listed conditions. In addition, conditions which promote hybridization under conditions of high stringency (e.g., increasing the temperature of the hybridization and/or wash steps, the use of formamide in the hybridization solution, etc.) may also be used.

As used herein, the term “hybridization” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C₀ t or R₀ t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, the term “Tₘ” is used in reference to the “melting temperature.” The melting temperature is the temperature at which a population of double-stranded nucleic acid molecules becomes half dissociated into single strands. As indicated by standard references, a simple estimate of the Tₘ value may be calculated by the equation: Tₘ=81.5+0.41 (% G+C), when a nucleic acid is in aqueous solution at 1M NaCl. Anderson et al., “Quantitative Filter Hybridization” In: Nucleic Acid Hybridization (1985). More sophisticated computations take structural, as well as sequence characteristics, into account for the calculation of Tₘ.

As used herein the term “stringency” is used in reference to the conditions of temperature, ionic strength, and the presence of other compounds such as organic solvents, under which nucleic acid hybridizations are conducted. “Stringency” typically occurs in a range from about Tₘ to about 20° C. to 25° C. below Tₘ. A “stringent hybridization” can be used to identify or detect identical polynucleotide sequences or to identify or detect similar or related polynucleotide sequences. For example, when fragments are employed in hybridization reactions under stringent conditions the hybridization of fragments which contain unique sequences (i.e., regions which are either non-homologous to or which contain less than about 50% homology or complementarity) are favored. Alternatively, when conditions of “weak” or “low” stringency are used hybridization may occur with nucleic acids that are derived from organisms that are genetically diverse (i.e., for example, the frequency of complementary sequences is usually low between such organisms).

As used herein, the term “amplifiable nucleic acid” is used in reference to nucleic acids which may be amplified by any amplification method. It is contemplated that “amplifiable nucleic acid” will usually comprise “sample template.”

As used herein, the term “sample template” refers to nucleic acid originating from a sample which is analyzed for the presence of a target sequence of interest. In contrast, “background template” is used in reference to nucleic acid other than sample template which may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

“Amplification” is defined as the production of additional copies of a nucleic acid sequence and is generally carried out either in vivo, i.e., for example by growing E. coli cells harboring recombinant (insert-containing) plasmid or fosmid vectors, or in vitro, i.e. for example using polymerase chain reaction. Dieffenbach C. W. and G. S. Dveksler (1995) In: PCR Primer, a Laboratory Manual, Cold Spring Harbor Press, Plainview, N.Y.

As used herein, the term “polymerase chain reaction” (“PCR”) refers to the method of K. B. Mullis U.S. Pat. Nos. 4,683,195 and 4,683,202, herein incorporated by reference, which describe a method for increasing the concentration of a segment of a target sequence in a mixture of genomic DNA without cloning or purification. The length of the amplified segment of the desired target sequence is determined by the relative positions of two oligonucleotide primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications. With PCR, it is also possible to amplify a complex mixture (library) of linear DNA molecules, provided they carry suitable universal sequences on either end such that universal PCR primers bind outside of the DNA molecules that are to be amplified.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxy-ribonucleotide. The primer must be sufficiently long to prime the synthesis of extension products in the presence of the inducing agent. The exact lengths of the primers will depend on many factors, including temperature, source of primer and the use of the method.

As used herein, the terms “restriction endonucleases” and “restriction enzymes” refer to bacterial enzymes, each of which cut double-stranded DNA at or near a specific nucleotide sequence.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

The term “in operable combination” as used herein, refers to any linkage of nucleic acid sequences in such a manner that the nucleic acid molecules are capable of performed a coordinated function.

The term “transfection” or “transfected” refers to the introduction of foreign DNA into a cell.

As used herein, the term “gene” means the deoxyribonucleotide sequences comprising the coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both amplified and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “polymerase chain reaction (PCR)” as used herein refers to a technique for amplifying a single or few copies of a nucleic acid sequence across several orders of magnitude. PCR relies on thermal cycling, comprising cycles of repeated heating and cooling of the reaction for nucleic acid melting and enzymatic replication of the DNA. Primers (short nucleic acid fragments) contain sequences complementary to the target region along with a polymerase enzyme (i.e., for example, Taq polymerase) As PCR progresses, the DNA generated is itself used as a template for replication, setting in motion a chain reaction in which the DNA template is exponentially amplified. PCR can be extensively modified to perform a wide array of genetic manipulations. U.S. Pat. No. 4,683,195 and U.S. Pat. No. 4,683,202, both of which are incorporated herein by reference.

The term “inverse polymerase chain reaction” as used herein refers to the amplification of a nucleic acid with only one known sequence. For example, when a DNA insert is circularized containing a known sequence, two primers may be designed to support PCR of the unknown sequence. See, FIG. 9. In contrast, conventional PCR requires two primers of known sequence that are complementary to both termini of the target DNA, but inverse PCR allows amplification to be carried out even if only one sequence is available from which primers may be designed.

The term “sequencing” as used herein refers to sequencing methods for determining the order of the nucleotide bases—adenine, guanine, cytosine, and thymine—in a nucleic acid molecule (e.g., a DNA nucleic acid molecule). Recent advances in the speed of sequencing (i.e., for example, high throughput sequencing) attained with modem DNA sequencing technology has been instrumental in the sequencing of the human genome, in the Human Genome Project.

### BRIEF DESCRIPTION OF THE FIGURES

The file of this patent contains at least one drawing executed in color. Copies of this patent with color drawings will be provided by the Patent and Trademark Office upon request and payment of the necessary fee.

FIG. 1 presents an illustrative schema for the construction of clonal sequencing libraries. Genomic DNA is fragmented into random pieces and cloned as a bacterial library. DNA from individual bacterial clones is sequenced and the sequence is assembled by using overlapping DNA regions.

FIG. 2 presents an exemplary illustration of a Sanger chain termination nucleic acid sequence ladder (gel electrophoresis) as compared to their representative fluorescent peaks.

FIG. 3 presents an illustrative description of the use of clonal libraries of DNA molecules that are compatible with an Illumina sequencing technology.

FIG. 4a illustrates a sequencing-by-synthesis technique used by an Illumina Genome Analyzer. Cluster strands created by bridge amplification are primed and all four fluorescently labeled, 3′-OH blocked nucleotides are added to the flow cell with DNA polymerase. The cluster strands are extended by one nucleotide.

FIG. 4b illustrates data collection from an Illumina Genome Analyzer following the incorporation step shown in FIG. 4a. The unused nucleotides and DNA polymerase molecules are washed away and a scan buffer is added to the flow cell. The optics system scans each lane of the flow cell by imaging units called tiles. Once imaging is completed, chemicals that effect cleavage of the fluorescent labels and the 3′-OH blocking groups are added to the flow cell, which prepares the cluster strands for another round of fluorescent nucleotide incorporation.

FIG. 5 presents one embodiment of a ligase-mediated sequencing approach compatible with a SOLiD sequencer (Applied Biosystems). DNA fragments are amplified on the surfaces of 1-μm magnetic beads to provide sufficient signal during the sequencing reactions, and are then deposited onto a flow cell slide. Ligase-mediated sequencing begins by annealing a primer to the shared adapter sequences on each amplified fragment, and then DNA ligase is provided along with specific fluorescent labeled 8mers, whose 4th and 5th bases are encoded by the attached fluorescent group. Each ligation step is followed by fluorescence detection, after which a regeneration step removes bases from the ligated 8mer (including the fluorescent group) and concomitantly prepares the extended primer for another round of ligation.

FIG. 6 presents representative principles of two-base encoding as performed in SOLiD sequencing. Because each fluorescent group on a ligated 8mer identifies a two-base combination, the resulting sequence reads can be screened for base-calling errors versus true polymorphisms versus single base deletions by aligning the individual reads to a known high-quality reference sequence.

FIG. 7 presents one embodiment for the creation of a pFosIll vector suitable for transfecting a microorganism for the generation of a large insert fosIll genomic library.

FIG. 8 presents one embodiment for the creation of a circularized jumping library comprising read pairs of <1 kb compatible with bridge amplification and/or emulsion amplification sequencing techniques.

FIG. 9 presents one embodiment of an inverse polymerase chain reaction.

FIG. 10 presents an embodiment of a modified F-plasmid-derived vector on the right (pFosIll-1, a fosmid cloning vector designed for generating ˜40-kb Illumina read pairs) along with the original fosmid cloning vector (pFos1; Kim et al., Nucleic Acids Res. 20:1083-1085 (1992); left vector).

FIG. 11 presents embodiments of a restriction map for a modified F-plasmid-derived vector (i.e., for example, pFosIll-1).

FIG. 11A: With a kanamycin stuffer sequence.

FIG. 11B: Without a kanamycin stuffer sequence.

FIG. 12 presents a close-up presentation of the polylinkers and nicking restriction sites in one embodiment of a modified F-plasmid (i.e., for example, pFossIll-1). In particular, a pair of useful restriction end nicking enzyme site comprises Nb.BbvCI, and flanks the Kanamycin stuffer sequence.

FIG. 13 presents one embodiment of excision the bulk of the cloned insert from a vector followed by re-circularization and co-ligation of the end-portions of the cloned insert, and inverse polymerase chain reaction that generates linear amplicons configured for bridge amplification high throughput sequencing.

FIG. 14 presents one embodiment showing the construction of pFOS1. Two NotI sites that directly flank the BamHI/HindIll cloning sites are not shown. Amp^{r} and CM^{r} indicate resistances to ampicillin and chloramphenicol, respectively.

FIG. 15 an embodiment of a modified F-plasmid-derived vector on the right (pFosIll-2, a fosmid cloning vector designed for generating ˜40-kb Illumina read pairs) along with the original fosmid cloning vector.

FIG. 16 presents one embodiment for the preparation of samples compatible with an Illumina sequencing platform.

FIG. 16A: DNA fragments are generated, for example, by random shearing and joined to a pair of oligonucleotides in a forked adaptor configuration. The ligated products are amplified using two oligonucleotide primers, resulting in double-stranded blunt-ended material with a different adaptor sequence on either end. The PCR product has the same structure and universal sequence attachments as the product of the inverse PCR shown in FIG. 13.

FIG. 16B: Formation of clonal single-molecule array. DNA fragments prepared as in FIG. 16A are denatured and single strands are annealed to complementary oligonucleotides on the flow cell surface (hatched). A new strand (dotted) is copied from the original strand in an extension reaction that is primed from the 3′ end of the surface bound oligonucleotide; the original strand is then removed by denaturation. The adaptor sequence at the 3′ end of each copied strand is annealed to a new surface-bound complementary oligonucleotide, forming a bridge and generating a new site for synthesis of a second strand (dotted). Multiple cycles of annealing, extension and denaturation in isothermal conditions result in growth of clusters, each,1 mm in physical diameter. Fedurco et al., “BTA, a novel reagent for DNA attachment on glass and efficient generation of solid-phase amplified DNA colonies” Nucleic Acids Res. 34: e22 (2006).

FIG. 16C: The DNA in each cluster is linearized by cleavage within one adaptor sequence (gap marked by an asterisk) and denatured, generating single-stranded template for sequencing by synthesis to obtain a sequence read (read 1; the sequencing product is dotted). To perform paired-read sequencing, the products of read 1 are removed by denaturation, the template is used to generate a bridge, the second strand is re-synthesized (shown dotted), and the opposite strand is then cleaved (gap marked by an asterisk) to provide the template for the second read (read 2).

FIG. 16D: Long-range paired-end sample preparation. To sequence the ends of a long (for example, 1 kb) DNA fragment, the ends of each fragment are tagged by incorporation of biotinylated (B) nucleotide and then circularized, forming a junction between the two ends. Circularized DNA is randomly fragmented and the biotinylated junction fragments are recovered and used as starting material in the standard sample preparation procedure illustrated in FIG. 16A. The orientation of the sequence reads relative to the DNA fragment is shown (magenta arrows). When aligned to the reference sequence, these reads are oriented with their 5′ ends towards each other (in contrast to the short insert paired reads produced as shown in FIGS. 16A-16C. Turquoise and blue lines represent oligonucleotides and red lines represent genomic DNA. All surface-bound oligonucleotides are attached to the flow cell by their 59 ends. Dotted lines indicate newly synthesized strands during cluster formation or sequencing.

FIG. 17 demonstrates the opposite primer pair configuration between sequencing large DNA inserts and short DNA inserts using an illustrative homozygous 3.6 kb deletion in NA18507 relative to the reference, supported by anomalously spaced long and short insert read pairs (orange) compared to regularly spaced read pairs (green). Note that the correct read orientation for long insert read pairs is different from that of the short insert read pairs and is denoted by the arrows which point in a 5′ to 3′ direction. See FIG. 16 for explanation. Note that this display is a composite of screen shots of the same window, overlapped for display purposes in this figure.

FIG. 18A presents exemplary data showing Illumina sequencing read pair results from an E. coli ˜40 kb fosIll jump library.

FIG. 18B presents exemplary data showing Illumina sequencing read pair results from an R. sphaeroides ˜40 kb fosIll jump library.

FIG. 19 presents an illustration of one embodiment of the construction of a fosIll jumping library.

FIG. 20 presents exemplary data showing a gel electrophoresis separation of pFosill(ΔKan) digests using Syber Green.

FIG. 21 presents one embodiment for the preparation of pFosill(ΔKan) vector arms.

FIG. 22 presents exemplary data showing a pulse field gel with 33 kb to 48 kb and 48 kb and 55 kb gel slices.

FIG. 23 presents exemplary data showing gel electrophoresis of Trial PCR fosIll fragments stained using SYBR Green I (10 ul in 100 mls water) for 10 min and viewed on a gel imager.

FIG. 24 presents an illustration of an ALLPATHS-LG algorithm process of fragment pair filling and artifacts.

FIG. 24A: The algorithm tries to close the black pair but finds another pair (red) that perfectly overlaps the black pair and closes its gap. Sequence from the red pair is inserted into the gap in the black pair, thus closing it.

FIG. 24B: The algorithm tries to close the black pair, but this time there is a SNP (A or T) between its gap. Two red pairs both overlap the black pair perfectly, providing two separate solutions to its closure, both of which are retained.

FIG. 25 illustrates the elimination of artifacts associated with sheared jumping libraries following, for example, the Illumina protocol. International Human Genome Sequencing Consortium, “Finishing the euchromatic sequence of the human genome” Nature 431:931-945 (2004).

FIG. 25A: DNA is sheared and size selected, yielding linear fragments.

FIG. 25B: The ends of these fragments are biotinylated and then the fragments are circularized and sheared.

FIG. 25C: Fragments of the circles are then enriched for those containing biotin where two reads enter from opposite sides but do not read the junction (arrows).

FIG. 25D: One of the reads passes through the junction point, creating a “chimeric” read.

FIG. 25E: The ends of a fragment that do not contain a junction point are read, yielding a read pair in opposite orientation to that show in FIG. 25C and whose true separation on the genome is small.

FIG. 26 presents one embodiment of a ShaRc fosmid sample preparation method.

FIG. 27 presents one example of post ShARC PCR Agilent electropherogram using a Three Spine Sticklebackfosmid genomic clone as source material.

FIG. 28 illustrate one embodiment of gap patching as executed by an ALLPATHS-LG genomic assembly algorithm.

FIG. 28A: Steps i-iv define a pool of oriented reads that might land in a given gap.

FIG. 28B: Steps v and vi define a stack of reads that align to a given read (top); the dotted line shows a column of the stack that “votes” to determine if the corresponding base on the given read is to be changed.

FIG. 28C: Step vii: All 16-mers that could be party to a bridge across the gap are found; dotted portions of reads are 16-mers that are excluded and then trimmed off the reads.

FIG. 28D: Steps viii-x: Closures of the gap are found by walking across the gap using perfect overlaps between the reads.

FIG. 29 presents exemplary data showing length distribution of genomic distance spanned by paired end sequences. Shown are smoothed histograms for the distribution of the spacing between unique read pairs from genomic DNA derived from Schizosaccharomyces pombe (FIG. 29A), Human K-562 cells (FIG. 29B), and Mouse C57Bl/6J cells (FIG. 29C). The y-axis is the percent of all unique read pairs that fall in the 1-kb bin indicated on the x-axis. The percentages of unique read pairs spanning 0 to 1 kb bin and 30 to 50 kb are indicated.

FIG. 30 compares three exemplary embodiments for introducing barcodes (e.g., barcode 375) into a fosIll library. Red P: Oligonucleotide phosphorylation. Grey P: Insert DNA phosphorylation.

FIG. 31 presents an illustration of one embodiment for converting a fosmid library into a jumping library that can be sequenced by Illumina. (a) the vector backbone with the two BbvCI sites and the binding sites for Illumina paired end sequencing primers ILMN-F and ILMN-R (blue and green arrows) is shown in black. The line representing the cloned insert is red on one end and blue on the other. At the end of the conversion process (f), the two ends of the insert are joined and flanked by four universal vector-derived bases (black), Illumina primers including the sequences required for bridge-amplification on the Illumina flow cell. The intermediates (b-e) steps are explained in the main text.

FIG. 32 illustrates an exemplary pfosIll cloning vector map. The restriction sites suitable for inserting the genomic DNA fragments (Eco72I, XcmI or BamHI) are flanked by forward and reverse Illumina-primer sequences (ILMN-F and ILMN-R) and two recognition sites for the nicking endonuclease Nb.BbvCI. The nicks are 5′ of the cloning site. The pUC-derived portion between the two cos sites is not present in the final circularized Fosmids which replicate under the control of oriS and the F-factor functions repE and sopA-C that ensure proper partition of the Fosmid among the two daughter cells.

FIG. 33 presents exemplary data showing the fosIll jump size distribution from mouse C57BL/6J cell template DNA.

FIG. 34 presents one embodiment of a pfosIll-3 sequence.

FIG. 35 presents one embodiment of a pfosIll-4 sequence.

### DETAILED DESCRIPTION OF THE INVENTION

The present invention is related to genomic nucleotide sequencing. In particular, the invention describes a paired end sequencing method that improves the yield of unique read pairs that are far (i.e., for example, ˜30-45 kb) apart in the genome. The method may use an inverse polymerase amplification, or shearing in combination with re-circularization, to convert a large-insert clone library (i.e., for example, a fosmid library) representing the genome to a plurality of linear amplification products (read pair jumping library) that are compatible with massively parallel sequencing techniques. The compositions and methods disclosed herein contemplate sequencing complex genomes as well as detecting chromosomal structural rearrangements.

Next-generation sequencing platforms such as the Illumina Genome Analyzer produces millions of short sequence reads. These reads can be pair wise, but construct size limitations restrict spacing of conventional read pairs to a few hundred bases. While there are protocols for in vitro construction of “jumping” libraries to enable read pairing over approximately 4-10 kb distances, it has been technically difficult to extend the range of in vitro jumps far beyond 10 kb. For traditional clone-based Sanger whole-genome sequencing, end-sequences of ˜40 kb fosmid or even-200 kb BAC clones provide the jumps necessary for providing long-range contiguity of genome assemblies.

However, none of them works well with DNA molecules much longer than 1 kb. Consequently, analyzing DNA fragments >1 kb by these technologies requires modern versions of the venerable “jumping” library whereby the ends of size-selected genomic DNA fragment are brought together by circularization, the bulk of the intervening DNA is excised and the junction fragment is isolated and sequenced. Collins et al., “Directional cloning of DNA fragments at a large distance from an initial probe: a circularization method” Proc Natl Acad Sci USA 1984, 81(21):6812-6816; Poustka et al., “Construction and use of human chromosome jumping libraries from NotI-digested DNA” Nature 1987, 325(6102):353-355.

Suitable protocols exist for all three 2nd generation sequencing platforms to convert DNA fragments into jumping libraries and to generate read pairs that span several kb of genomic distance. Such jumps, in combination with paired-end reads from fragment libraries, have enabled high-quality draft assemblies of microbial genomes from massively parallel sequencing data. Maccallum et al., “ALLPATHS 2: small genomes assembled accurately and with high continuity from short paired reads” Genome Biol 2009, 10(10):R103. However, recent assemblies of mammalian end human genomes turned out highly fragmented—despite jumps up to ˜12 kb in length. Li et al., “The sequence and de novo assembly of the giant panda genome” Nature 2010, 463(7279):311-317; Li et al., “De novo assembly of human genomes with massively parallel short read sequencing” Genome Res 2010, 20(2):265-272; and Schuster et al., “Complete Khoisan and Bantu genomes from southern Africa” Nature 2010, 463(7283):943-947. Without the equivalent of fosmid or BAC end sequences, the N50 scaffold lengths were one or two orders of magnitude below that of traditional clone-based draft assemblies of mammalian genomes. Lindblad-Toh et al., “Genome sequence, comparative analysis and haplotype structure of the domestic dog” Nature 2005, 438(7069):803-819; and Mikkelsen et al., “Genome of the marsupial Monodelphis domestica reveals innovation in non-coding sequences” Nature 2007, 447(7141):167-177.

The data presented herein demonstrate the successful next-generation pair wise end-sequencing of one embodiment of fosmid libraries (i.e., for example, fosmid libraries cloned in a specially modified pFosill vector) by an Illumina platform. Fosmid libraries in pFosill vectors, like other fosmid libraries, may be packaged in bacteriophage X heads, circularized and amplified in E. coli. Unlike other jumping libraries, the present invention contemplates that clonal DNA insert size selection and circularization, the most challenging steps of making long-distance jump libraries, can be carried out in vivo. Although it is not necessary to understand the mechanism of an invention, it is believed that carrying out the size selection and circularization of the genomic DNA fragment in vivo library improves efficiency and maximizes the yield of productive and unique read pairs. For example, to convert a cloned circular molecule containing a 40 kb genomic DNA fragment into an Illumina-sequenceable amplicon, E. coli DNA polymerase I may be used to translate two endonuclease nicks (introduced at predefined sites at the respective vector termini) to regions on either side of the cloned insert. Subsequent digest with nuclease S1 at the translated nicks releases the bulk of the cloned insert leaving only the terminal portions (typically less than 1 kb each) of the fragments attached to the cloning vector. A subsequent second circularization reaction ligates the terminal insert portions remaining attached to the vector. The co-ligated terminal-portions are then selectively amplified using inverse inverse polymerase chain reaction, resulting in the production of linear insert amplicons compatible with bridge and/or emulsion amplification sequencing techniques.

### I. Conventional Jumping Libraries

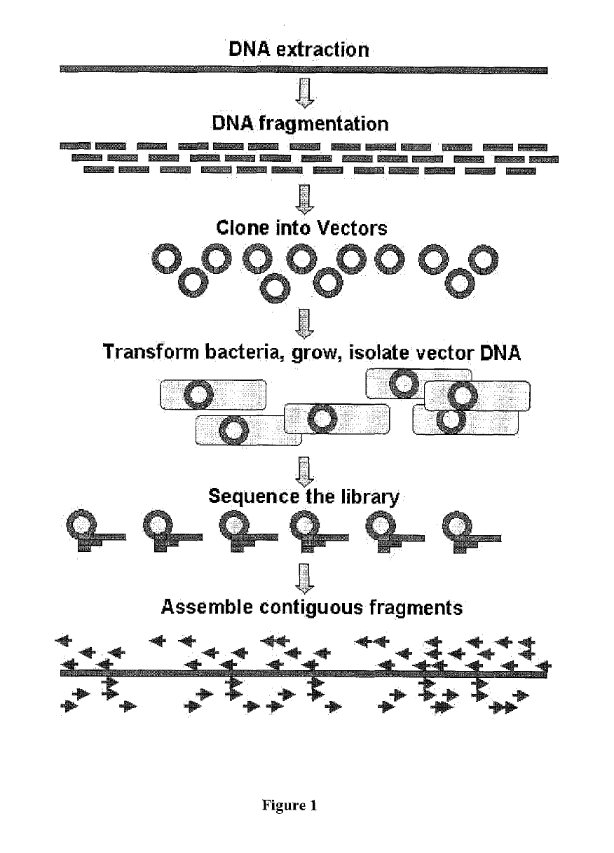

In molecular biology, a library is generally understood as a collection of DNA fragments that is stored and propagated in a population of microorganisms (i.e., for example, E. coli) through the process of molecular cloning. Several different types of DNA libraries have been reported, including, but not limited to, amplified libraries that are formed from reverse-transcribed RNA and genomic libraries that formed from fragmented genomic DNA. DNA library technology has been developed for many different applications depending upon the source of the original DNA fragments. Further, there are differences in cloning vectors and techniques used in library preparation but, in general, each DNA fragment is uniquely inserted into a cloning vector, wherein a pool of recombinant DNA molecules are then transferred into a population of microorganisms. On average, each microorganism contains one nucleotide construct (i.e., for example, a vector comprising a nucleotide fragment insert). As the population of microorganisms is grown in culture, the DNA inserts are replicated as the microorganisms propagate (i.e., for example, cloned). See, FIG. 1.

To support next-generation sequencing, various in vitro techniques were introduced that generated libraries often referred to as a “jumping”, “large-insert gap” or “mate-pair” libraries. Typically, genome fragments are circularized wherein both ends of an original genome fragment are brought together, and the ligation junction marked by a biotin tag. These labeled fragments were then subjected to either random shearing, restriction digestion or other fragmentation methods. The labeled amplicons were then isolated using streptavidin, ligated to universal adapters and PCR amplified. The PCR product (“jumping library”) then undergoes either paired-end sequencing or complete single end sequencing.

Conventional jump techniques performed in vitro have distinct disadvantages because they become increasingly difficult and inefficient as the insert (“jump”) size increases. In particular, these cloning techniques encounter a significant loss in genetic material during the library-construction process which requires multiple lossy steps such as electrophoretic size selection and a circularization by a dilute ligation reaction, capture of biotinylated junction fragments and adapter ligation. In particular, in vitro jumping has not been reported to work well for jump sizes approaching 40 kb. Even moderately long (i.e., for example, 15-25 kb) in vitro jumping libraries tend to incur heavy nucleotide loss during in vitro manipulations such as size-selection and circularization before the first amplification step (the PCR). It has been estimated that for 10-kb jumping libraries the loss of genetic material before the final PCR amplification step is in the order of 10⁴ fold. The loss is even higher for longer-distance jumping libraries. Attempts to generate ˜20-kb read pairs in this manner have often resulted in less than 1 million unique read pairs. Because source genomic DNA for genome sequencing is usually limited (i.e., for example to less than 100 μg of genetic material) losses of this magnitude result in a reduction of library complexity, such that the subsequent sequencing process cannot generate a sufficient number of unique independent read pairs and sequencing depth required for the assembly process.

### II. FosIll Jumping Libraries

Constructing a jumping library entails numerous physical and enzymatic DNA manipulations. Several steps, particularly size selection and circularization of genomic DNA fragments, become increasingly difficult and inefficient as the desired jump length goes up. The cumulative losses during the process ultimately limit the number of unique jumps in the library. Thus, to our knowledge, no jumping library constructed to date solely in vitro matched the average span and complexity of a traditional fosmid library.

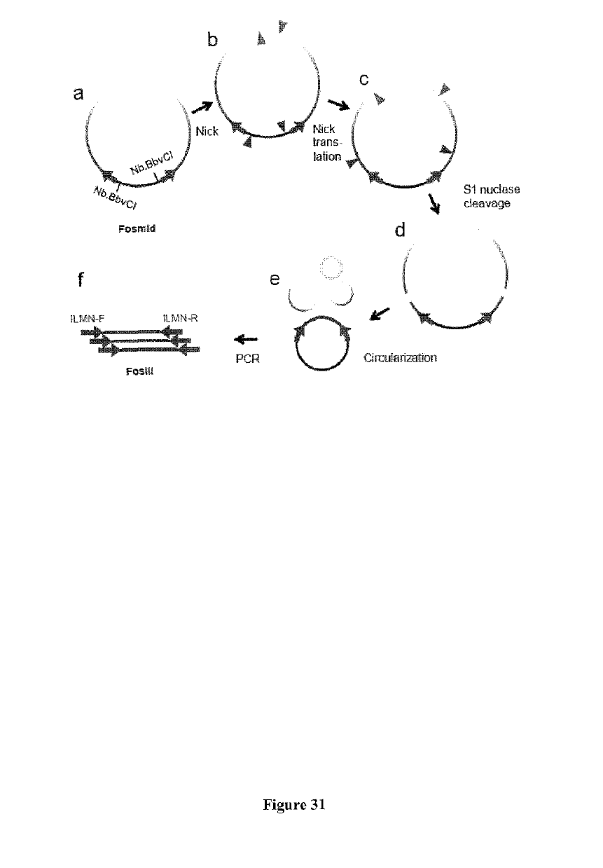

Avoiding bacterial cloning steps has been believed to improve the efficiency of massively parallel sequencing technologies. However, until embodiments of the present invention were disclosed, technical challenges prevented the production of equivalents to Fosmid or BAC-end sequences that were known to provide long-range linking information for assembling and analyzing complex genomes during the Sanger-based sequencing era. In some embodiments, the present invention contemplates the construction of “next generation” Fosmid-end sequences. In one embodiment, a method comprises converting Fosmid libraries into Illumina-compatible “fosIll” libraries. See. FIG. 31.

For example, the 2 nicks are propagated into the insert via nick translation (step a). A Nb.BbvCI enzyme then introduces the 2 nicks in the vector (step b). An S1 nuclease digestion excises the bulk of the insert (step c). The vector with the genomic insert termini attached is re-circularized (step d). Inverse PCR with Illumina enrichment primers renders the sequencing-ready fosIll library (step e). The fosmid library is amplified in liquid culture and DNA is prepared (step f).

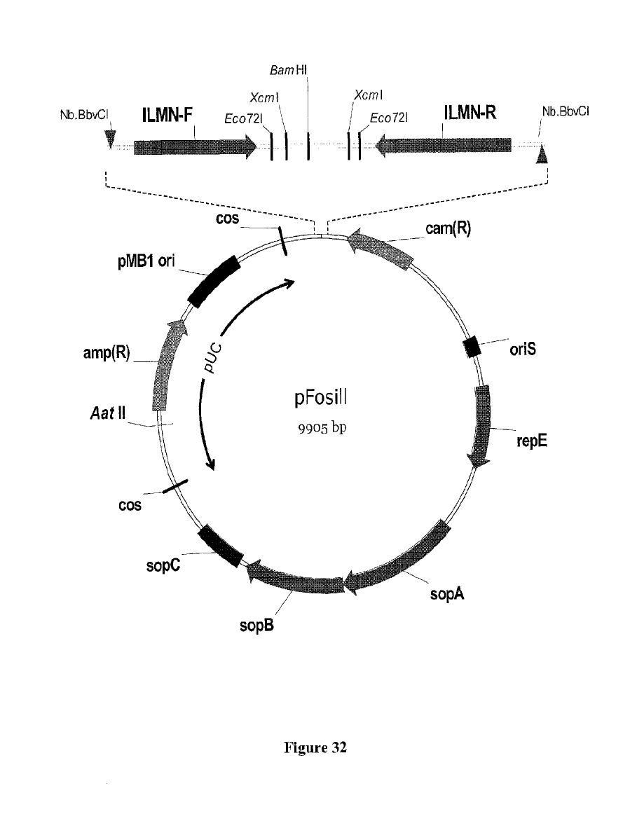

Briefly, to generate “next generation” fosmid-end sequences, some embodiments described herein convert fosmid libraries into Illumina-compatible fosIll libraries. For example, Illumina sequencing primer sites were inserted next to a cloning site of pFos1 plasmid. To facilitate the conversion of fosmid libraries to Illumina-compatible fosIll jumping libraries, Illumina forward and reverse sequencing primers sites were incorporated into the fosmid vector pFos1. Kim et al., “Stable propagation of cosmid sized human DNA inserts in an F factor based vector” Nucleic Acids Res 1992, 20(5):1083-1085. A modified cloning site comprises blunt-end fragments produced by random shearing and end-repair can be inserted between two Eco72I sites, four bases downstream of each sequencing primer. See, FIG. 32. Alternatively, fragments with a single 3′-dG overhang can be inserted between the two XcmI sites. Godiska et al., “Linear plasmid vector for cloning of repetitive or unstable sequences in Escherichia coli” Nucleic Acids Res 2010, 38(6):e88. BamHI sites for cloning fragments were generated by traditional partial digestion with MboI. The cloning site is flanked by two recognition sites for Nb.BbvCI, a modified restriction enzyme that cleaves only one strand at its recognition site. The modified vector, called pfosIll, retains the salient features of pFOS1 such as dual cos sites and a pUC-derived pMB1 origin of replication which facilitates the production of large amounts of pFosill plasmid. Evans et al., “High efficiency vectors for cosmid microcloning and genomic analysis” Gene 1989, 79(1):9-20. To prepare the vector for blunt-end cloning, the plasmid is digested with AatII and Eco72I. The resulting vector arms are dephosphorylated and ligated to blunt-end phosphorylated fragments that have been size-selected to ˜35 to 45 kb. In vitro packaging removes the pUC-derived portion of the plasmid, thereby rendering a single-copy amplicon under the control of the F-factor origin of replication oriS.

fosmid libraries were then constructed and amplified in liquid culture. To excise the bulk of each cloned ˜40-kb fragment, a nicking restriction endonuclease was used to introduce two single-strand nicks flanking the cloned inserts. The nicks were translated a few hundred by into the inserts where they were fully cleaved by S1 nuclease. Re-circularization of the vector with the genomic insert termini attached followed by inverse PCR with standard Illumina enrichment primers renders the sequencing-ready fosIll library. Starting with 30 μg of DNA, this method can generate more than 5 million unique fosIll jumps that span 38 kb on average (s.d. 3.5 kb).

fosIll's providing ˜80-fold physical coverage of the mouse genome improved the long-range connectivity of Illumina-based ALLPATH-LG draft de novo assemblies (infra). The N50 scaffold length was 17.4 Mb, rivaling the scaffold length (16.9 Mb) of the capillary-sequencing-based draft assembly. fosIll's are also used to identify and map a variety of gross chromosomal structural abnormalities in the human K562 chronic myelogenous leukemia cell line. For example, a t(9;22) translocation that gives rise to the BCR-ABL1 fusion protein was bracketed by more than 800 distinct supporting read pairs. The exact breakpoint was pinpointed by multiple split read alignments crossing the 9;22 junction. Two additional rearrangements detected by fosIll jumps corroborated gene fusions previously identified by RNA-seq.

The two BbvCI sites in the vector are oriented such that digestion with Nb.BbvCI introduces two single-strand nicks, each located 5′ of the cloning site. See, FIG. 31(b) and FIG. 32. DNA polymerase I, in the presence of dNTPs, therefore translates the two nicks towards and into the cloned insert. where it can be fully cleaved by S1 nuclease to produce a double-strand break within a few hundred by from the cloning vector. See, FIG. 31(c) and FIG. 31(d), respectively. Fragment ends are polished to facilitate subsequent blunt-end ligation. This series of steps is analogous to the nick-translation-directed cleavage used to construct jumping libraries for SOLiD sequencing. McKernan et al., “Sequence and structural variation in a human genome uncovered by short-read, massively parallel ligation sequencing using two-base encoding” Genome Res 2009, 19(9):1527-1541. Of note, BbvCI sites in the cloned genome fragment will be nicked as well and thus give rise to genomic DNA fragments that are no longer attached to the vector.

After blunt-end ligation dilute conditions favor circularization wherein the “jumps” (i.e., for example, the co-ligated ends of the cloned insert) are flanked by Illumina forward and reverse sequencing primer binding sites and can be amplified by PCR using standard full-length Illumina paired-end enrichment primers that include the sequences necessary for cluster amplification. See, FIG. 31(e) and FIG. 31(t), respectively. The size range of the products is wide but can be controlled to some degree by varying the duration of the nick-translation. A final size selection on a preparative gel renders the sequence-ready fosIll library.

In one embodiment, the present invention contemplates a method comprising constructing a fosmid library utilizing a modified pFOS1 cloning vector compatible with next-generation paired-end sequencing platforms (i.e., for example, a fosmid in a modified pfosIll cloning vector). Although it is not necessary to understand the mechanism of an invention, it is believed that pFOS1 is a bireplicon plasmid, and exists in high copy number in microorganisms (i.e., for example, E. coli) due to the pUC replication origin. However, the pUC-derived portion of the plasmid is removed during in vitro packaging after ligating the arms to insert DNA, rendering the vector with a DNA insert under the control of F factor replication origin.

In one embodiment, the present invention contemplates a method for generating 10⁶ to 10⁷ independent fosmid clones from less than 100 μg of input genomic DNA. In one embodiment, the method further comprises amplifying the primary fosmid library by overnight liquid culture and maxi-prepping, thereby propogating the microbial clones and the incorporated vectors. Once the primary fosmid library is propogated, DNA losses do not necessarily affect the complexity of the jumping library when subsequently amplified by polymerase chain reaction. Thus, it is possible to generate 10⁶ to 10⁷ independent unique long-distance read pairs (i.e., for example, 30-45 kb inserts) from less than 100 μg of input genomic DNA. Although it is not necessary to understand the mechanism of an invention, it is believed that high yield long-distance read pairs provide linking information that may be useful for de novo genome assembly and/or to scan a large and complex genome such as the human genome for gross structural rearrangement such as translocations, inversion, deletions, insertions.

In one embodiment, the present invention contemplates a method for converting a conventional bacterial large-insert clone library into a sequence-ready jumping library that is compatible with massively parallel sequencing technology. In one embodiment, this conversion may comprise modifying a bacterial cloning vector incorporated with a nucleic acid sequence insert. In one embodiment, the cloning vector comprises a cloning site comprising at least two restriction enzyme recognition site clusters, flanked by a universal primer pair and an endonuclease site pair. In other embodiments, the clonal library conversion further comprises; i) nicking the endonuclease site pair; ii) translating the two nicks several hundred bases into the cloned insert followed by S1 nuclease digestion to generate a double-strand break at the site of the translated nick; iii) re-circularization and inverse PCR amplification of the co-ligated end portions of the cloned insert; and iv) sequencing on next-generation sequencing platforms (i.e., for example, Illumina).

Unlike conventional in vitro jumping library sequencing techniques, the present invention contemplates recircularizing and amplifying a sequenceable DNA insert fragment using in such a manner that affinity isolation using biotin capture on streptavidin coated solid phases to select for a junction fragment is not necessary. In particular, the primers used for the inverse PCR reaction are the same primers that are used for a standard (non-jumping) fragment library for next-generation sequencing. Thus, the product of the inverse PCR reaction can be used directly for (i.e., for example, is compatible with) a bridge amplification sequencing technique (i.e., for example, Illumina) or an emulsion amplification sequence technique (i.e., for example, SOLiD).

In some embodiments, the present invention contemplates a composition comprising an F-plasmid-derived fosmid cloning vector having terminal next-generation sequencing primer pairs flanking the cloning site. In one embodiment, the fosmid carries a large DNA insert. In one embodiment, the primer pairs are bridge amplification primer pairs (i.e., for example, Illumina). In one embodiment, the primer pairs are emulsion amplification primer pairs (i.e., for example, SOLiD). In one embodiment, the cloning site is flanked by a nicking endonuclease site pair. In one embodiment, the large DNA insert ranges between approximately 10-1,000 kb, preferably between 20-500 kb, and more preferably between approximately 25-200 kb, and most preferably between approximately 30-45 kb. In one embodiment, the large DNA insert is approximately 40 kb.

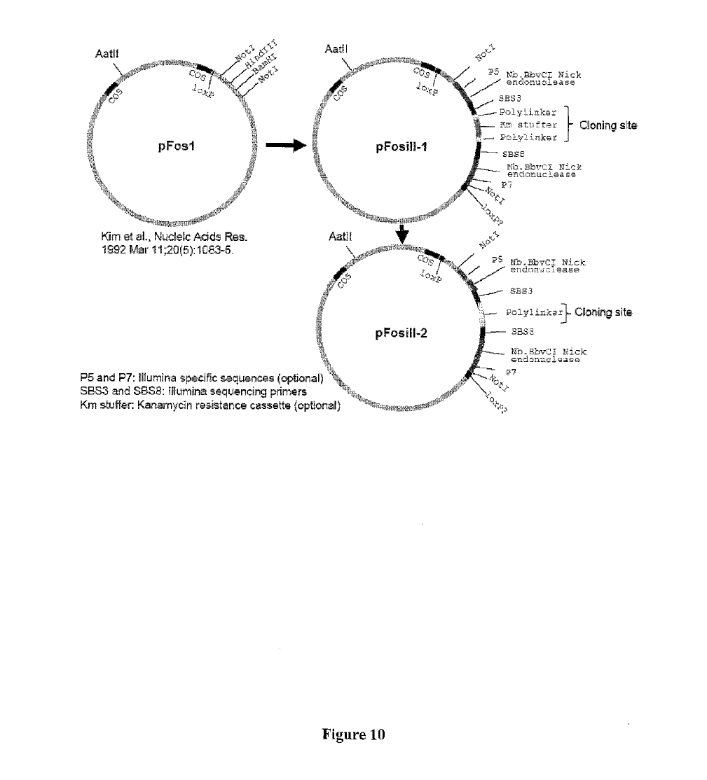

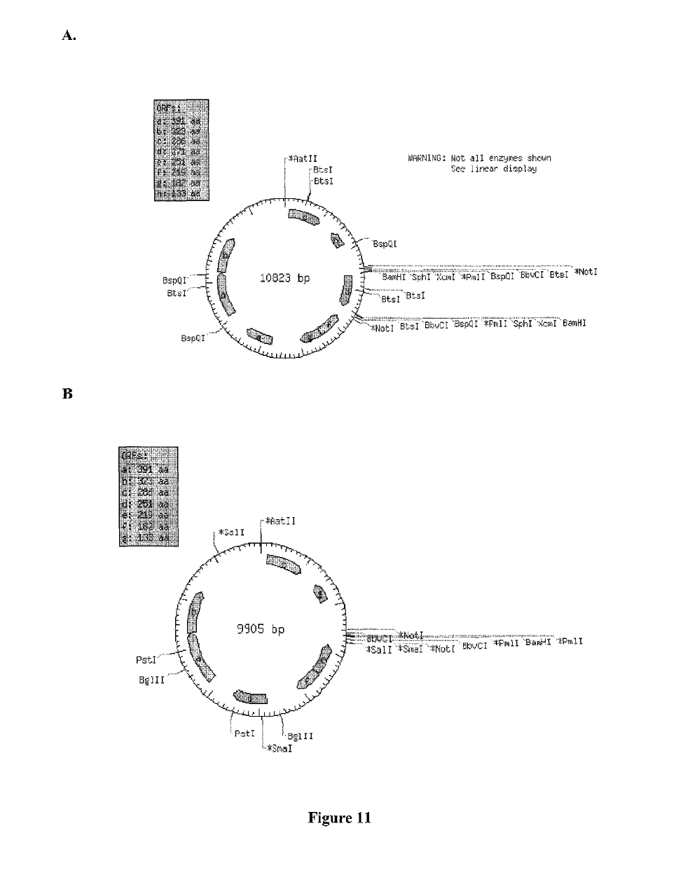

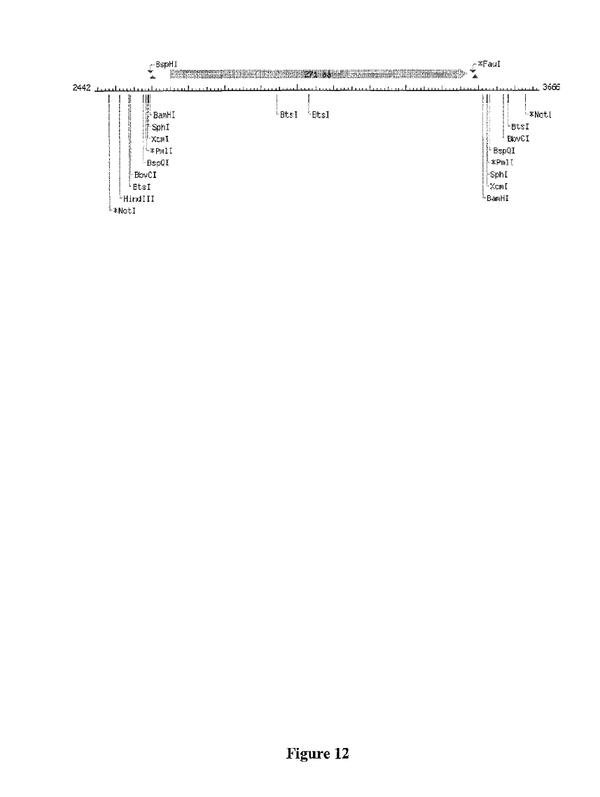

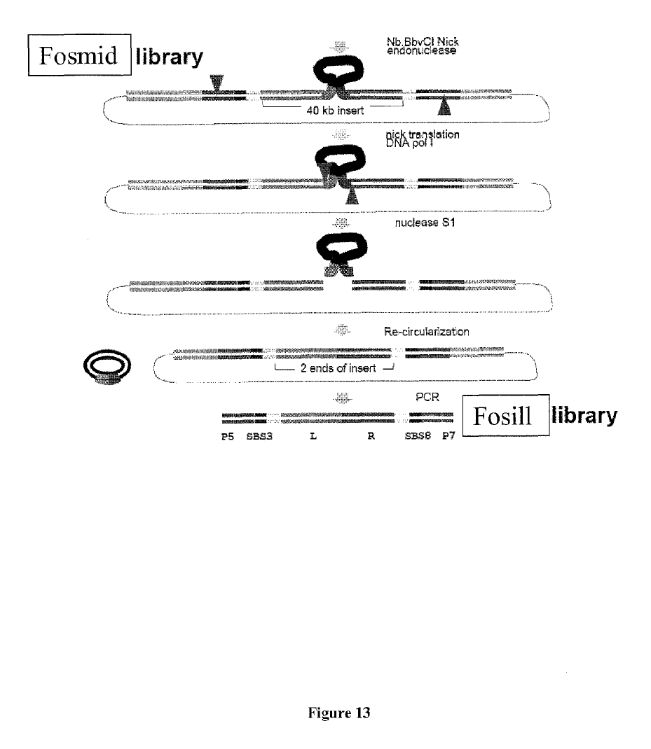

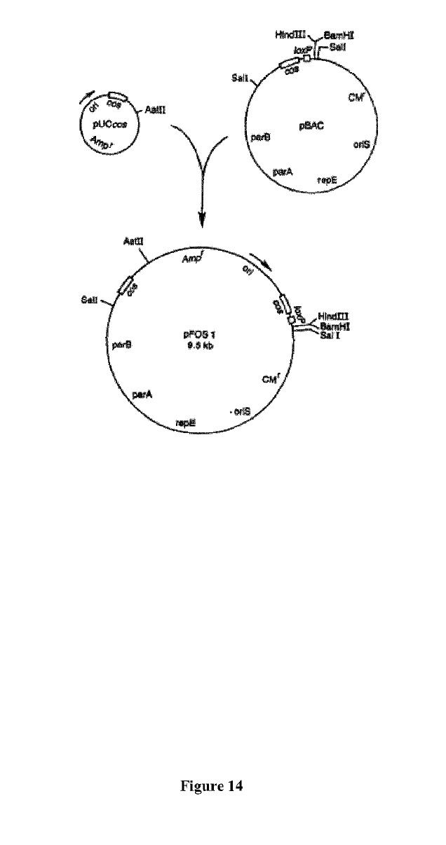

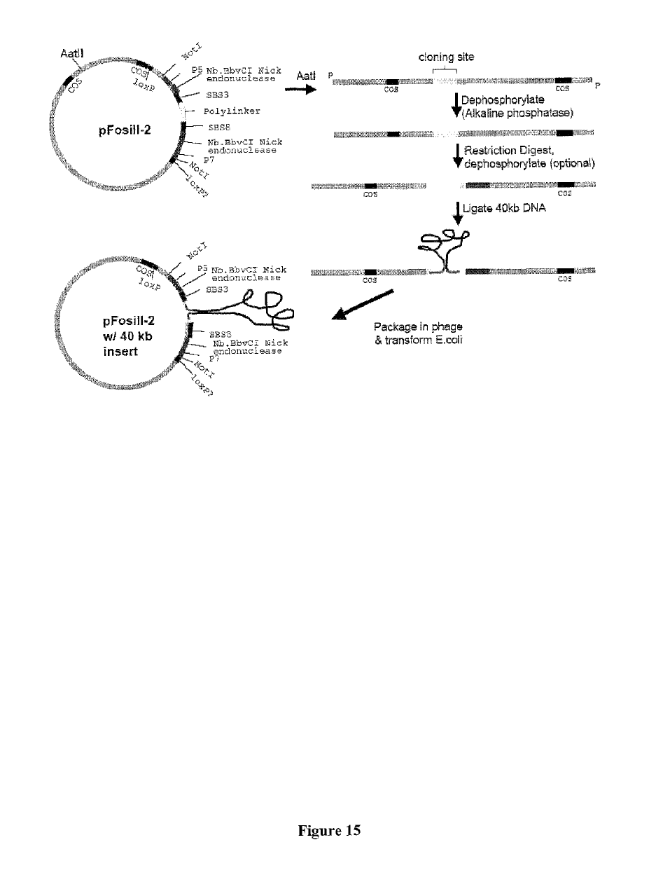

In one embodiment, the fosmid is incorporated into a microorganism (i.e., for example, E. coli), thereby creating a fosmid library. In one embodiment, the F-plasmid comprises a modified pFOS1 cloning vector having at least two polylinkers that flank a kanamycin resistance gene cassette as a stuffer fragment. In one embodiment, the polylinkers are identical. See, FIG. 10. In one embodiment, the vector does not have a kanamycin resistance gene cassette. See, FIG. 11B. In one embodiment, the polylinker comprises a DNA duplex having the sequences of:

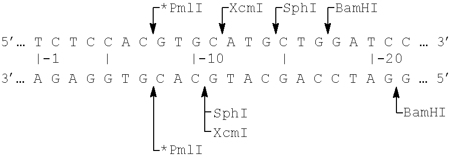

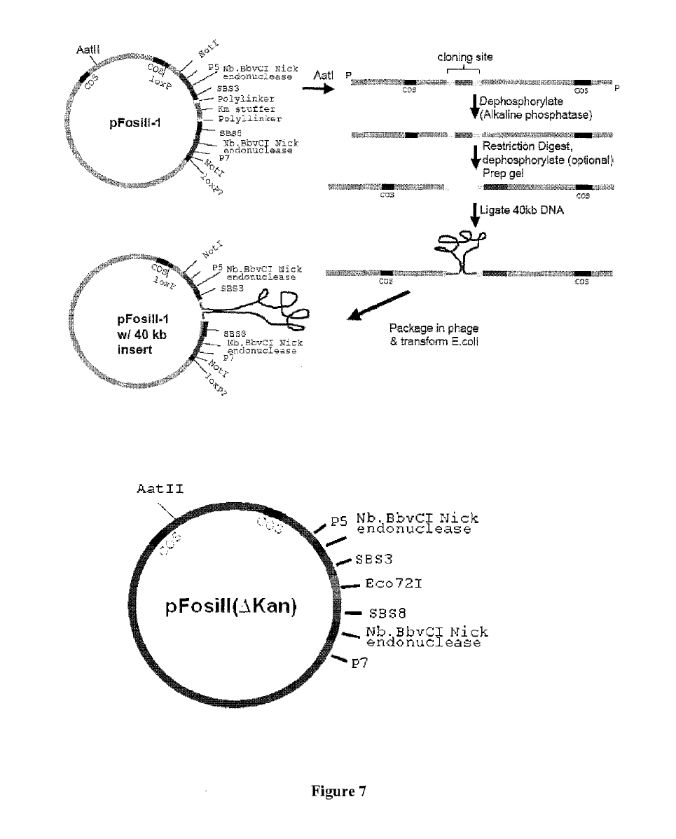

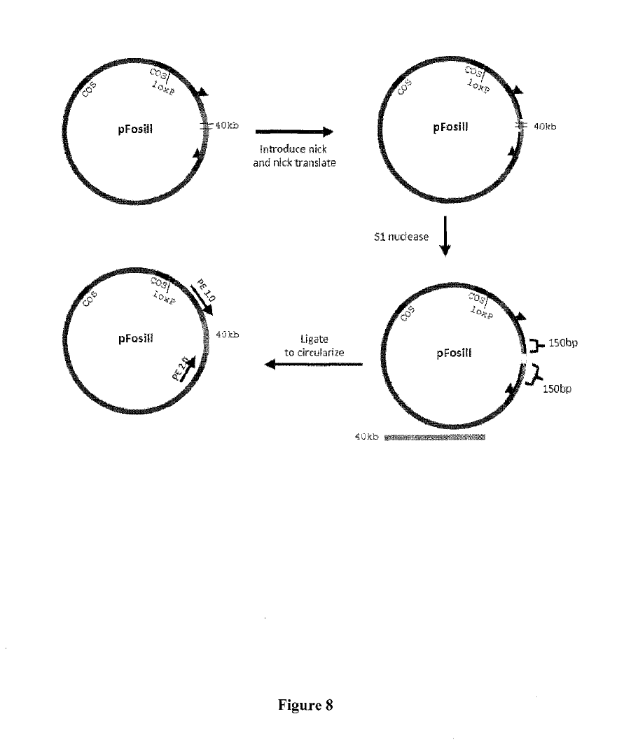

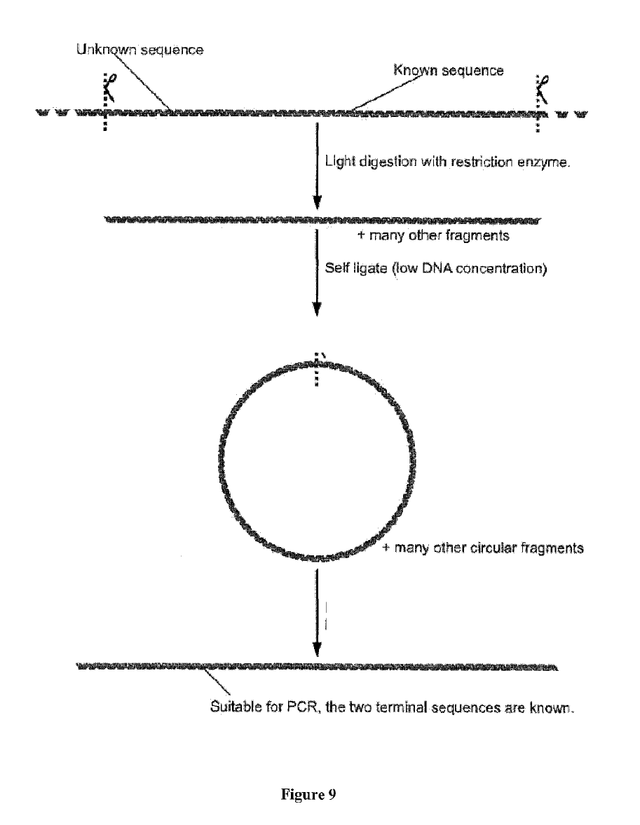

In one embodiment, the cloning sites comprise a PmII restriction site for cloning blunt end fragments. In one embodiment, the PmII restriction site is cleaved with Eco72I. In one embodiment, the cloning sites comprise a XcmI restriction site for cloning fragments with 3′ G overhang. In one embodiment, the cloning sites comprise a SphI restriction site for cloning partially digested NlaIII fragments with 4 bp 3′ overhang. In one embodiment, the cloning sites comprise a BamHI for cloning partially digested MboI fragments with 4 bp 5′ overhang. In one embodiment, the cloning site is flanked by SBS3 and SBS8 sequences, the Illumina sequencing primer sequences for the forward and reverse read, respectively, and two Nb.BbvC1 nicking endonuclease sites, thereby creating a pfosIll cloning vector (i.e., for example, a fosmid cloning vector that contains primer sequences useful in Illumina sequencing). See, FIG. 7. In one embodiment, the modified pFOS1 cloning vector comprises a pfosIll-1 vector having the nucleotide sequence of:

(SEQ ID NO: 1)
GTCAGGTGGCACTTTTCGGGGAAATGTGCGCGGAACCCCTATTTGTTTAT
TTTTCTAAATACATTCAAATATGTATCCGCTCATGAGACAATAACCCTGA
TAAATGCTTCAATAATATTGAAAAAGGAAGAGTATGAGTATTCAACATTT
CCGTGTCGCCCTTATTCCCTTTTTTGCGGCATTTTGCCTTCCTGTTTTTG
CTCACCCAGAAACGCTGGTGAAAGTAAAAGATGCTGAAGATCAGTTGGGT
GCACGAGTGGGTTACATCGAACTGGATCTCAACAGCGGTAAGATCCTTGA
GAGTTTTCGCCCCGAAGAACGTTTTCCAATGATGAGCACTTTTAAAGTTC
TGCTATGTGGCGCGGTATTATCCCGTATTGACGCCGGGCAAGAGCAACTC
GGTCGCCGCATACACTATTCTCAGAATGACTTGGTTGAGTACTCACCAGT
CACAGAAAAGCATCTTACGGATGGCATGACAGTAAGAGAATTATGCAGTG
CTGCCATAACCATGAGTGATAACACTGCGGCCAACTTACTTCTGACAACG
ATCGGAGGACCGAAGGAGCTAACCGCTTTTTTGCACAACATGGGGGATCA
TGTAACTCGCCTTGATCGTTGGGAACCGGAGCTGAATGAAGCCATACCAA
ACGACGAGCGTGACACCACGATGCCTGTAGCAATGGCAACAACGTTGCGC
AAACTATTAACTGGCGAACTACTTACTCTAGCTTCCCGGCAACAATTAAT
AGACTGGATGGAGGCGGATAAAGTTGCAGGACCACTTCTGCGCTCGGCCC
TTCCGGCTGGCTGGTTTATTGCTGATAAATCTGGAGCCGGTGAGCGTGGG
TCTCGCGGTATCATTGCAGCACTGGGGCCAGATGGTAAGCCCTCCCGTAT
CGTAGTTATCTACACGACGGGGAGTCAGGCAACTATGGATGAACGAAATA
GACAGATCGCTGAGATAGGTGCCTCACTGATTAAGCATTGGTAACTGTCA
GACCAAGTTTACTCATATATACTTTAGATTGATTTAAAACTTCATTTTTA
ATTTAAAAGGATCTAGGTGAAGATCCTTTTTGATAATCTCATGACCAAAA
TCCCTTAACGTGAGTTTTCGTTCCACTGAGCGTCAGACCCCGTAGAAAAG
ATCAAAGGATCTTCTTGAGATCCTTTTTTTCTGCGCGTAATCTGCTGCTT
GCAAACAAAAAAACCACCGCTACCAGCGGTGGTTTGTTTGCCGGATCAAG
AGCTACCAACTCTTTTTCCGAAGGTAACTGGCTTCAGCAGAGCGCAGATA
CCAAATACTGTCCTTCTAGTGTAGCCGTAGTTAGGCCACCACTTCAAGAA
CTCTGTAGCACCGCCTACATACCTCGCTCTGCTAATCCTGTTACCAGTGG
CTGCTGCCAGTGGCGATAAGTCGTGTCTTACCGGGTTGGACTCAAGACGA
TAGTTACCGGATAAGGCGCAGCGGTCGGGCTGAACGGGGGGTTCGTGCAC
ACAGCCCAGCTTGGAGCGAACGACCTACACCGAACTGAGATACCTACAGC
GTGAGCATTGAGAAAGCGCCACGCTTCCCGAAGGGAGAAAGGCGGACAGG
TATCCGGTAAGCGGCAGGGTCGGAACAGGAGAGCGCACGAGGGAGCTTCC
AGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGG
AAAAACGCCAGCAACGCGGCCTTTTTACGGTTCCTGGCCTTTTGCTGGCC
TTTTGCTCACATGTTCTTTCCTGCGTTATCCCCTGATTCTGTGGATAACC
GTATTACCGCCTTTGAGTGAGCTGATACCGCTCGCCGCAGCCGAACGACC
GAGCGCAGCGAGTCAGTGAGCGAGGAAGCGGAAGAGCGCCCAATACGCAA
ACCGCCTCTCCCCGCGCGTTGGCCGATTCATTAATGCAGCTTGCGCCGTT
CCACTTCGATGCGTCAGTGAAGCGACATGAGGTTGCCCCGTATTCAGTGT
CGCTGATTTGTATTGTCTGAAGTTGTTTTTACGTTAAGTTGATGCAGATC
AATTAATACGATACCTGCGTCATAATTGATTATTTGACGTGGTTTGATGG
CCTCCACGCACGTTGTGATATGTAGATGATAATCATTATCACTTTACGGG
TCCTTTCCGGTGATCCGACAGGTTACGGGGCGGCGACCTCGCGGGTTTTC
GCTATTTATGAAAATTTTCCGGTTTAAGGCGTTTCCGTTCTTCTTCGTCA
TAACTTAATGTTTTTATTTAAAATACCCTCTGAAAAGAAAGGAAACGACA
GGTGCTGAAAGCGAGCTTTTTGGCCTCTGTCGTTTCCTTTCTCTGTTTTT
GTCCGTGGAATGAACAATGGAAGTCCGAGCTCATCGCTAATAACTTCGTA
TAGCATACATTATACGAAGTTATATTCGATGCGGCCGCTAATACGACTCA
CTATAGGGAGAAGCTTAATGATACGGCGACCACCGACACTGCTGAGGACA
CTCTTTCCCTACACGACGCTCTTCCGATCTCCACGTGCATGCTGGATCCA
TCATGAACAATAAAACTGTCTGCTTACATAAACAGTAATACAAGGGGTGT
TATGAGCCATATTCAACGGGAAACGTCTTGCTCGAGGCCGCGATTAAATT
CCAACATGGATGCTGATTTATATGGGTATAAATGGGCTCGCGATAATGTC
GGGCAATCAGGTGCGACAATCTATCGATTGTATGGGAAGCCCGATGCGCC
AGAGTTGTTTCTGAAACATGGCAAAGGTAGCGTTGCCAATGATGTTACAG
ATGAGATGGTCAGACTAAACTGGCTGACGGAATTTATGCCTCTTCCGACC
ATCAAGCATTTTATCCGTACTCCTGATGATGCATGGTTACTCACCACTGC
GATCCCCGGGAAAACAGCATTCCAGGTATTAGAAGAATATCCTGATTCAG
GTGAAAATATTGTTGATGCGCTGGCAGTGTTCCTGCGCCGGTTGCATTCG
ATTCCTGTTTGTAATTGTCCTTTTAACAGCGATCGCGTATTTCGTCTCGC
TCAGGCGCAATCACGAATGAATAACGGTTTGGTTGATGCGAGTGATTTTG
ATGACGAGCGTAATGGCTGGCCTGTTGAACAAGTCTGGAAAGAAATGCAT
AAACTTTTGCCATTCTCACCGGATTCAGTCGTCACTCATGGTGATTTCTC
ACTTGATAACCTTATTTTTGACGAGGGGAAATTAATAGGTTGTATTGATG
TTGGACGAGTCGGAATCGCAGACCGATACCAGGATCTTGCCATCCTATGG
AACTGCCTCGGTGAGTTTTCTCCTTCATTACAGAAACGGCTTTTTCAAAA
ATATGGTATTGATAATCCTGATATGAATAAATTGCAGTTTCATTTGATGC
TCGATGAGTTTTTCTAATCAGAATTGGTTAATTAGCCCGCCTAATGAGCG
GGCTTTTTTTTGGATCCAGCATGCACGTGGAGATCGGAAGAGCGGTTCAG
CAGGAATGCCGAGACCGATCCTCAGCAGTGTCGTATGCCGTCTTCTGCTT
GAGATCCTATAGTGTCACCTAAATCGTATGCGGCCGCCCGGGCCGTCGAC
CAATTCTCATGTTTGACAGCTTATCATCGAATTTCTGCCATTCATCCGCT
TATTATCACTTATTCAGGCGTAGCACCAGGCGTTTAAGGGCACCAATAAC
TGCCTTAAAAAAATTACGCCCCGCCCTGCCACTCATCGCAGTACTGTTGT
AATTCATTAAGCATTCTGCCGACATGGAAGCCATCACAGACGGCATGATG
AACCTGAATCGCCAGCGGCATCAGCACCTTGTCGCCTTGCGTATAATATT
TGCCCATGGTGAAAACGGGGGCGAAGAAGTTGTCCATATTGGCCACGTTT
AAATCAAAACTGGTGAAACTCACCCAGGGATTGGCTGAGACGAAAAACAT
ATTCTCAATAAACCCTTTAGGGAAATAGGCCAGGTTTTCACCGTAACACG
CCACATCTTGCGAATATATGTGTAGAAACTGCCGGAAATCGTCGTGGTAT
TCACTCCAGAGCGATGAAAACGTTTCAGTTTGCTCATGGAAAACGGTGTA
ACAAGGGTGAACACTATCCCATATCACCAGCTCACCGTCTTTCATTGCCA
TACGGAATTCCGGATGAGCATTCATCAGGCGGGCAAGAATGTGAATAAAG
GCCGGATAAAACTTGTGCTTATTTTTCTTTACGGTCTTTAAAAAGGCCGT
AATATCCAGCTGAACGGTCTGGTTATAGGTACATTGAGCAACTGACTGAA
ATGCCTCAAAATGTTCTTTACGATGCCATTGGGATATATCAACGGTGGTA
TATCCAGTGATTTTTTTCTCCATTTTAGCTTCCTTAGCTCCTGAAAATCT
CGATAACTCAAAAAATACGCCCGGTAGTGATCTTATTTCATTATGGTGAA
AGTTGGAACCTCTTACGTGCCGATCAACGTCTCATTTTCGCCAAAAGTTG
GCCCAGGGCTTCCCGGTATCAACAGGGACACCAGGATTTATTTATTCTGC
GAAGTGATCTTCCGTCACAGGTATTTATTCGCGATAAGCTCATGGAGCGG
CGTAACCGTCGCACAGGAAGGACAGAGAAAGCGCGGATCTGGGAAGTGAC
GGACAGAACGGTCAGGACCTGGATTGGGGAGGCGOTTGCCGCCGCTGCTG
CTGACGGTGTGACGTTCTCTGTTCCGGTCACACCACATACGTTCCGCCAT
TCCTATGCGATGCACATGCTGTATGCCGGTATACCGCTGAAAGTTCTGCA
AAGCCTGATGGGACATAAGTCCATCAGTTCAACGGAAGTCTACACGAAGG
TTTTTGCGCTGGATGTGGCTGCCCGGCACCGGGTGCAGTTTGCGATGCCG
GAGTCTGATGCGGTTGCGATGCTGAAACAATTATCCTGAGAATAAATGCC
TTGGCCTTTATATGGAAATGTGGAACTGAGTGGATATGCTGTTTTTGTCT
GTTAAACAGAGAAGCTGGCTGTTATCCACTGAGAAGCGAACGAAACAGTC
GGGAAAATCTCCCATTATCGTAGAGATCCGCATTATTAATCTCAGGAGCC
TGTGTAGCGTTTATAGGAAGTAGTGTTCTGTCATGATGCCTGCAAGCGGT
AACGAAAACGATTTGAATATGCCTTCAGGAACAATAGAAATCTTCGTGCG
GTGTTACGTTGAAGTGGAGCGGATTATGTCAGCAATGGACAGAACAACCT
AATGAACACAGAACCATGATGTGGTCTGTCCTTTTACAGCCAGTAGGCTC
GCCGCAGTCGAGCGACGGCGAAGCCCTCGAGTGAGCGAGGAAGCACCAGG
GAACAGCACTTATATATTCTGCTTACACACGATGCCTGAAAAAACTTCCC
TTGGGGTTATCCACTTATCCACGGGGATATTTTTATAATTATTTTTTTTA
TAGTTTTTAGATCTTCTTTTTTAGAGCGCCTTGTAGGCCTTTATCCATGC
TGGTTCTAGAGAAGGTGTTGTGACAAATTGCCCTTTCAGTOTGACAAATC
ACCCTCAAATGACAGTCCTGTCTGTGACAAATTGCCCTTAACCCTGTGAC
AAATTGCCCTCAGAAGAAGCTGTTTTTTCACAAAGTTATCCCTGCTTATT
GACTCTTTTTTATTTAGTGTGACAATCTAAAAACTTGTCACACTTCACAT
GGATCTGTCATGGCGGAAACAGCGGTTATCAATCACAAGAAACGTAAAAA
TAGCCCGCGAATCGTCCAGTCAAACGACCTCACTGAGGCGGCATATAGTC
TCTCCCGGGATCAAAAACGTATGCTGTATCTGTTCGTTGACCAGATCAGA
AAATCTGATGGCACCCTACAGGAACATGACGGTATCTGCGAGATCCATGT
TGCTAAATATGCTGAAATATTCGGATTGACCTCTGCGGAAGCCAGTAAGG
ATATACGGCAGGCATTGAAGAGTTTCGCGGGGAAGGAAGTGGTTTTTTAT
CGCCCTGAAGAGGATGCCGGCGATGAAAAAGGCTATGAATCTTTTCCTTG
GTTTATCAAACGTGCGCACAGTCCATCCAGAGGGCTTTACAGTGTACATA
TCAACCCATATCTCATTCCCTTCTTTATCGGGTTACAGAACCGGTTTACG
CAGTTTCGGCTTAGTGAAACAAAAGAAATCACCAATCCGTATGCCATGCG
TTTATACGAATCCCTGTGTCAGTATCGTAAGCCGGATGGCTCAGGCATCG
TCTCTCTGAAAATCGACTGGATCATAGAGCGTTACCAGCTGCCTCAAAGT
TACCAGCGTATGCCTGACTTCCGCCGCCGCTTCCTGCAGGTCTGTGTTAA
TGAGATCAACAGCAGAACTCCAATGCGCCTCTCATACATTGAGAAAAAGA
AAGGCCGCCAGACGACTCATATCGTATTTTCCTTCCGCGATATCACTTCC
ATGACGACAGGATAGTCTGAGGGTTATCTGTCACAGATTTGAGGGTGGTT
CGTCACATTTGTTCTGACCTACTGAGGGTAATTTGTCACAGTTTTGCTGT
TTCCTTCAGCCTGCATGGATTTTCTCATACTTTTTGAACTGTAATTTTTA
AGGAAGCCAAATTTGAGGGCAGTTTGTCACAGTTGATTTCCTTCTCTTTC
CCTTCGTCATGTGACCTGATATCGGGGGTTAGTTCGTCATCATTGATGAG
GGTTGATTATCACAGTTTATTACTCTGAATTGGCTATCCGCGTGTGTACC
TCTACCTGGAGTTTTTCCCACGGTGGATATTTCTTCTTGCGCTGAGCGTA
AGAGCTATCTGACAGAACAGTTCTTCTTTGCTTCCTCGCCAGTTCGCTCG
CTATGCTCGGTTACACGGCTGCGGCGAGCGCTAGTGATAATAAGTGACTG
AGGTATGTGCTCTTCTTATCTCCTTTTGTAGTGTTGCTCTTATTTTAAAC
AACTTTGCGGTTTTTTGATGACTTTGCGATTTTGTTGTTGCTTTGCAGTA
AATTGCAAGATTTAATAAAAAAACGCAAAGCAATGATTAAAGGATGTTCA
GAATGAAACTCATGGAAACACTTAACCAGTGCATAAACGCTGGTCATGAA
ATGACGAAGGCTATCGCCATTGCACAGTTTAATGATGACAGCCCGGAAGC
GAGGAAAATAACCCGGCGCTGGAGAATAGGTGAAGCAGCGGATTTAGTTG
GGGTTTCTTCTCAGGCTATCAGAGATGCCGAGAAAGCAGGGCGACTACCG
CACCCGGATATGGAAATTCGAGGACGGGTTGAGCAACGTGTTGGTTATAC
AATTGAACAAATTAATCATATGCGTGATGTGTTTGGTACGCGATTGCGAC
GTGCTGAAGACGTATTTCCACCGGTGATCGGGGTTGCTGCCCATAAAGGT
GGCGTTTACAAAACCTCAGTTTCTGTTCATCTTGCTCAGGATCTGGCTCT
GAAGGGGCTACGTGTTTTGCTCGTGGAAGGTAACGACCCCCAGGGAACAG
CCTCAATGTATCACGGATGGGTACCAGATCTTCATATTCATGCAGAAGAC
ACTCTCCTGCCTTTCTATCTTGGGGAAAAGGACGATGTCACTTATGCAAT
AAAGCCCACTTGCTGGCCGGGGCTTGACATTATTCCTTCCTGTCTGGCTC
TGCACCGTATTGAAACTGAGTTAATGGGCAAATTTGATGAAGGTAAACTG
CCCACCGATCCACACCTGATGCTCCGACTGGCCATTGAAACTGTTGCTCA
TGACTATGATGTCATAGTTATTGACAGCGCGCCTAACCTGGGTATCGGCA
CGATTAATGTCGTATGTGCTGCTGATGTGCTGATTGTTCCCACGCCTGCT
GAGTTGTTTGACTACACCTCCGCACTGCAGTTTTTCGATATGCTTCGTGA
TCTGCTCAAGAACGTTGATCTTAAAGGGTTCGAGCCTGATGTACGTATTT
TGCTTACCAAATACAGCAATAGTAATGGCTCTCAGTCCCCGTGGATGGAG
GAGCAAATTCGGGATGCCTGGGGAAGCATGGTTCTAAAAAATGTTGTACG
TGAAACGGATGAAGTTGGTAAAGGTCAGATCCGGATGAGAACTGTTTTTG
AACAGGCCATTGATCAACGCTCTTCAACTGGTGCCTGGAGAAATGCTCTT
TCTATTTGGGAACCTGTCTGCAATGAAATTTTCGATCGTCTGATTAAACC
ACGCTGGGAGATTAGATAATGAAGCGTGCGCCTGTTATTCCAAAACATAC
GCTCAATACTCAACCGGTTGAAGATACTTCGTTATCGACACCAGCTGCCC
CGATGGTGGATTCGTTAATTGCGCGCGTAGGAGTAATGGCTCGCGGTAAT
GCCATTACTTTGCCTGTATGTGGTCGGGATGTGAAGTTTACTCTTGAAGT
GCTCCGGGGTGATAGTGTTGAGAAGACCTCTCGGGTATGGTCAGGTAATG
AACGTGACCAGGAGCTGCTTACTGAGGACGCACTGGATGATCTCATCCCT
TCTTTTCTACTGACTGGTCAACAGACACCGGCGTTCGGTCGAAGAGTATC
TGGTGTCATAGAAATTGCCGATGGGAGTCGCCGTCGTAAAGCTGCTGCAC
TTACCGAAAGTGATTATCGTGTTCTGGTTGGCGAGCTGGATGATGAGCAG
ATGGCTGCATTATCCAGATTGGGTAACGATTATCGCCCAACAAGTGCTTA
TGAACGTGGTCAGCGTTATGCAAGCCGATTGCAGAATGAATTTGCTGGAA
ATATTTCTGCGCTGGCTGATGCGGAAAATATTTCACGTAAGATTATTACC
CGCTGTATCAACACCGCCAAATTGCCTAAATCAGTTGTTGCTCTTTTTTC
TCACCCCGGTGAACTATCTGCCCGGTCAGGTGATGCACTTCAAAAAGCCT
TTACAGATAAAGAGGAATTACTTAAGCAGCAGGCATCTAACCTTCATGAG
CAGAAAAAAGCTGGGGTGATATTTGAAGCTGAAGAAGTTATCACTCTTTT
AACTTCTGTGCTTAAAACGTCATCTGCATCAAGAACTAGTTTAAGCTCAC
GACATCAGTTTGCTCCTGGAGCGACAGTATTGTATAAGGGCGATAAAATG
GTGCTTAACCTGGACAGGTCTCGTGTTCCAACTGAGTGTATAGAGAAAAT
TGAGGCCATTCTTAAGGAACTTGAAAAGCCAGCACCCTGATGCGACCACG
TTTTAGTCTACGTTTATCTGTCTTTACTTAATGTCCTTTGTTACAGGCCA
GAAAGCATAACTGGCCTGAATATTCTCTCTGGGCCCACTGTTCCACTTGT
ATCGTCGGTCTGATAATCAGACTGGGACCACGGTCCCACTCGTATCGTCG
GTCTGATTATTAGTCTGGGACCACGGTCCCACTCGTATCGTCGGTCTGAT
TATTAGTCTGGGACCACGGTCCCACTCGTATCGTCGGTCTGATAATCAGA
CTGGGACCACGGTCCCACTCGTATCGTCGGTCTGATTATTAGTCTGGGAC
CATGGTCCCACTCGTATCGTCGGTCTGATTATTAGTCTGGGACCACGGTC
CCACTCGTATCGTCGGTCTGATTATTAGTCTGGAACCACGGTCCCACTCG
TATCGTCGGTCTGATTATTAGTCTGGGACCACGGTCCCACTCGTATCGTC
GGTCTGATTATTAGTCTGGGACCACGATCCCACTCGTGTTGTCGGTCTGA
TTATCGGTCTGGGACCACGGTCCCACTTGTATTGTCGATCAGACTATCAG
CGTGAGACTACGATTCCATCAATGCCTGTCAAGGGCAAGTATTGACATGT
CGTCGTAACCTGTAGAACGGAGTAACCTCGGTGTGCGGTTGTATGCCTGC
TGTGGATTGCTGCTGTGTCCTGCTTATCCACAACATTTTGCGCACGGTTA
TGTGGACAAAATACCTGGTTACCCAGGCCGTGCCGGCACGTTAACCGGGC
TGCATCCGATGCAAGTGTGTCGCTGTCGACGAGCTCGCGAGCTCGGACAT
GAGGTTGCCCCGTATTCAGTGTCGCTGATTTGTATTGTCTGAAGTTGTTT
TTACGTTAAGTTGATGCAGATCAATTAATACGATACCTGCGTCATAATTG
ATTATTTGACGTGGTTTGATGGCCTCCACGCACGTTGTGATATGTAGATG
ATAATCATTATCACTTTACGGGTCCTTTCCGGTGATCCGACAGGTTACGG
GGCGGCGACCTCGCGGGTTTTCGCTATTTATGAAAATTTTCCGGTTTAAG
GCGTTTCCGTTCTTCTTCGTCATAACTTAATGTTTTTATTTAAAATACCC
TCTGAAAAGAAAGGAAACGACAGGTGCTGAAAGCGAGGCTTTTTGGCCTC
TGTCGTTTCCTTTCTCTGTTTTTGTCCGTGGAATGAACAATGGAAGTCCT
GGCGTAATAGCGAAGAGGCCCGCACCGATCGCCCTTCCCAACAGTTGCGC
AGCCTGAATGGCGAATGGCGCCTGATGCGGTATTTTCTCCTTACGCATCT
GTGCGGTATTTCACACCGCATATGGTGCACTCTCAGTACAATCTGCTCTG
ATGCCGCATAGTTAAGCCAGCCCCGACACCCGCCAACACCCGCTGACGCG
CCCTGACGGGCTTGTCTGCTCCCGGCATCCGCTTACAGACAAGCTGTGAC
CGTCTCCGGGAGCTGCATGTGTCAGAGGTTTTCACCGTCATCACCGAAAC
GCGCGAGACGAAAGGGCCTCGTGATACGCCTATTTTTATAGGTTAATGTC
ATGATAATAATGGTTTCTTAGAC.

In one embodiment, the modified pFOS1 cloning vector comprises a pfosIll-2 vector having the nucleotide sequence of:

(SEQ ID NO: 2)
GTCAGGTGGCACTTTTCGGGGAAATGTGCGCGGAACCCCTATTTGTTTAT
TTTTCTAAATACATTCAAATATGTATCCGCTCATGAGACAATAACCCTGA
TAAATGCTTCAATAATATTGAAAAAGGAAGAGTATGAGTATTCAACATTT
CCGTGTCGCCCTTATTCCCTTTTTTGCGGCATTTTGCCTTCCTGTTTTTG
CTCACCCAGAAACGCTGGTGAAAGTAAAAGATGCTGAAGATCAGTTGGGT
GCACGAGTGGGTTACATCGAACTGGATCTCAACAGCGGTAAGATCCTTGA
GAGTTTTCGCCCCGAAGAACGTTTTCCAATGATGAGCACTTTTAAAGTTC
TGCTATGTGGCGCGGTATTATCCCGTATTGACGCCGGGCAAGAGCAACTC
GGTCGCCGCATACACTATTCTCAGAATGACTTGGTTGAGTACTCACCAGT
CACAGAAAAGCATCTTACGGATGGCATGACAGTAAGAGAATTATGCAGTG
CTGCCATAACCATGAGTGATAACACTGCGGCCAACTTACTTCTGACAACG
ATCGGAGGACCGAAGGAGCTAACCGCTTTTTTGCACAACATGGGGGATCA
TGTAACTCGCCTTGATCGTTGGGAACCGGAGCTGAATGAAGCCATACCAA
ACGACGAGCGTGACACCACGATGCCTGTAGCAATGGCAACAACGTTGCGC
AAACTATTAACTGGCGAACTACTTACTCTAGCTTCCCGGCAACAATTAAT
AGACTGGATGGAGGCGGATAAAGTTGCAGGACCACTTCTGCGCTCGGCCC
TTCCGGCTGGCTGGTTTATTGCTGATAAATCTGGAGCCGGTGAGCGTGGG
TCTCGCGGTATCATTGCAGCACTGGGGCCAGATGGTAAGCCCTCCCGTAT
CGTAGTTATCTACACGACGGGGAGTCAGGCAACTATGGATGAACGAAATA
GACAGATCGCTGAGATAGGTGCCTCACTGATTAAGCATTGGTAACTGTCA
GACCAAGTTTACTCATATATACTTTAGATTGATTTAAAACTTCATTTTTA
ATTTAAAAGGATCTAGGTGAAGATCCTTTTTGATAATCTCATGACCAAAA
TCCCTTAACGTGAGTTTTCGTTCCACTGAGCGTCAGACCCCGTAGAAAAG
ATCAAAGGATCTTCTTGAGATCCTTTTTTTCTGCGCGTAATCTGCTGCTT
GCAAACAAAAAAACCACCGCTACCAGCGGTGGTTTGTTTGCCGGATCAAG
AGCTACCAACTCTTTTTCCGAAGGTAACTGGCTTCAGCAGAGCGCAGATA
CCAAATACTGTCCTTCTAGTGTAGCCGTAGTTAGGCCACCACTTCAAGAA
CTCTGTAGCACCGCCTACATACCTCGCTCTGCTAATCCTGTTACCAGTGG
CTGCTGCCAGTGGCGATAAGTCGTGTCTTACCGGGTTGGACTCAAGACGA
TAGTTACCGGATAAGGCGCAGCGGTCGGGCTGAACGGGGGGTTCGTGCAC
ACAGCCCAGCTTGGAGCGAACGACCTACACCGAACTGAGATACCTACAGC
GTGAGCATTGAGAAAGCGCCACGCTTCCCGAAGGGAGAAAGGCGGACAGG
TATCCGGTAAGCGGCAGGGTCGGAACAGGAGAGCGCACGAGGGAGCTTCC
AGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGG
AAAAACGCCAGCAACGCGGCCTTTTTACGGTTCCTGGCCTTTTGCTGGCC
TTTTGCTCACATGTTCTTTCCTGCGTTATCCCCTGATTCTGTGGATAACC
GTATTACCGCCTTTGAGTGAGCTGATACCGCTCGCCGCAGCCGAACGACC
GAGCGCAGCGAGTCAGTGAGCGAGGAAGCGGAAGAGCGCCCAATACGCAA
ACCGCCTCTCCCCGCGCGTTGGCCGATTCATTAATGCAGCTTGCGCCGTT
CCACTTCGATGCGTCAGTGAAGCGACATGAGGTTGCCCCGTATTCAGTGT
CGCTGATTTGTATTGTCTGAAGTTGTTTTTACGTTAAGTTGATGCAGATC
AATTAATACGATACCTGCGTCATAATTGATTATTTGACGTGGTTTGATGG
CCTCCACGCACGTTGTGATATGTAGATGATAATCATTATCACTTTACGGG
TCCTTTCCGGTGATCCGACAGGTTACGGGGCGGCGACCTCGCGGGTTTTC
GCTATTTATGAAAATTTTCCGGTTTAAGGCGTTTCCGTTCTTCTTCGTCA
TAACTTAATGTTTTTATTTAAAATACCCTCTGAAAAGAAAGGAAACGACA
GGTGCTGAAAGCGAGCTTTTTGGCCTCTGTCGTTTCCTTTCTCTGTTTTT
GTCCGTGGAATGAACAATGGAAGTCCGAGCTCATCGCTAATAACTTCGTA
TAGCATACATTATACGAAGTTATATTCGATGCGGCCGCTAATACGACTCA
CTATAGGGAGAAGCTTAATGATACGGCGACCACCGACACTGCTGAGGACA
CTCTTTCCCTACACGACGCTCTTCCGATCTCCACGTGCATGCTGGATCCA
GCATGCACGTGGAGATCGGAAGAGCGGTTCAGCAGGAATGCCGAGACCGA
TCCTCAGCAGTGTCGTATGCCGTCTTCTGCTTGAGATCCTATAGTGTCAC
CTAAATCGTATGCGGCCGCCCGGGCCGTCGACCAATTCTCATGTTTGACA
GCTTATCATCGAATTTCTGCCATTCATCCGCTTATTATCACTTATTCAGG
CGTAGCACCAGGCGTTTAAGGGCACCAATAACTGCCTTAAAAAAATTACG
CCCCGCCCTGCCACTCATCGCAGTACTGTTGTAATTCATTAAGCATTCTG
CCGACATGGAAGCCATCACAGACGGCATGATGAACCTGAATCGCCAGCGG
CATCAGCACCTTGTCGCCTTGCGTATAATATTTGCCCATGGTGAAAACGG
GGGCGAAGAAGTTGTCCATATTGGCCACGTTTAAATCAAAACTGGTGAAA
CTCACCCAGGGATTGGCTGAGACGAAAAACATATTCTCAATAAACCCTTT
AGGGAAATAGGCCAGGTTTTCACCGTAACACGCCACATCTTGCGAATATA
TGTGTAGAAACTGCCGGAAATCGTCGTGGTATTCACTCCAGAGCGATGAA
AACGTTTCAGTTTGCTCATGGAAAACGGTGTAACAAGGGTGAACACTATC
CCATATCACCAGCTCACCGTCTTTCATTGCCATACGGAATTCCGGATGAG
CATTCATCAGGCGGGCAAGAATGTGAATAAAGGCCGGATAAAACTTGTGC
TTATTTTTCTTTACGGTCTTTAAAAAGGCCGTAATATCCAGCTGAACGGT
CTGGTTATAGGTACATTGAGCAACTGACTGAAATGCCTCAAAATGTTCTT
TACGATGCCATTGGGATATATCAACGGTGGTATATCCAGTGATTTTTTTC
TCCATTTTAGCTTCCTTAGCTCCTGAAAATCTCGATAACTCAAAAAATAC
GCCCGGTAGTGATCTTATTTCATTATGGTGAAAGTTGGAACCTCTTACGT
GCCGATCAACGTCTCATTTTCGCCAAAAGTTGGCCCAGGGCTTCCCGGTA
TCAACAGGGACACCAGGATTTATTTATTCTGCGAAGTGATCTTCCGTCAC
AGGTATTTATTCGCGATAAGCTCATGGAGCGGCGTAACCGTCGCACAGGA
AGGACAGAGAAAGCGCGGATCTGGGAAGTGACGGACAGAACGGTCAGGAC
CTGGATTGGGGAGGCGGTTGCCGCCGCTGCTGCTGACGGTGTGACGTTCT
CTGTTCCGGTCACACCACATACGTTCCGCCATTCCTATGCGATGCACATG
CTGTATGCCGGTATACCGCTGAAAGTTCTGCAAAGCCTGATGGGACATAA
GTCCATCAGTTCAACGGAAGTCTACACGAAGGTTTTTGCGCTGGATGTGG
CTGCCCGGCACCGGGTGCAGTTTGCGATGCCGGAGTCTGATGCGGTTGCG
ATGCTGAAACAATTATCCTGAGAATAAATGCCTTGGCCTTTATATGGAAA
TGTGGAACTGAGTGGATATGCTGTTTTTGTCTGTTAAACAGAGAAGCTGG
CTGTTATCCACTGAGAAGCGAACGAAACAGTCGGGAAAATCTCCCATTAT
CGTAGAGATCCGCATTATTAATCTCAGGAGCCTGTGTAGCGTTTATAGGA
AGTAGTGTTCTGTCATGATGCCTGCAAGCGGTAACGAAAACGATTTGAAT
ATGCCTTCAGGAACAATAGAAATCTTCGTGCGGTGTTACGTTGAAGTGGA
GCGGATTATGTCAGCAATGGACAGAACAACCTAATGAACACAGAACCATG
ATGTGGTCTGTCCTTTTACAGCCAGTAGGCTCGCCGCAGTCGAGCGACGG
CGAAGCCCTCGAGTGAGCGAGGAAGCACCAGGGAACAGCACTTATATATT
CTGCTTACACACGATGCCTGAAAAAACTTCCCTTGGGGTTATCCACTTAT
CCACGGGGATATTTTTATAATTATTTTTTTTATAGTTTTTAGATCTTCTT
TTTTAGAGCGCCTTGTAGGCCTTTATCCATGCTGGTTCTAGAGAAGGTGT
TGTGACAAATTGCCCTTTCAGTGTGACAAATCACCCTCAAATGACAGTCC
TGTCTGTGACAAATTGCCCTTAACCCTGTGACAAATTGCCCTCAGAAGAA
GCTGTTTTTTCACAAAGTTATCCCTGCTTATTGACTCTTTTTTATTTAGT
GTGACAATCTAAAAACTTGTCACACTTCACATGGATCTGTCATGGCGGAA
ACAGCGGTTATCAATCACAAGAAACGTAAAAATAGCCCGCGAATCGTCCA
GTCAAACGACCTCACTGAGGCGGCATATAGTCTCTCCCGGGATCAAAAAC
GTATGCTGTATCTGTTCGTTGACCAGATCAGAAAATCTGATGGCACCCTA
CAGGAACATGACGGTATCTGCGAGATCCATGTTGCTAAATATGCTGAAAT
ATTCGGATTGACCTCTGCGGAAGCCAGTAAGGATATACGGCAGGCATTGA
AGAGTTTCGCGGGGAAGGAAGTGGTTTTTTATCGCCCTGAAGAGGATGCC
GGCGATGAAAAAGGCTATGAATCTTTTCCTTGGTTTATCAAACGTGCGCA
CAGTCCATCCAGAGGGCTTTACAGTGTACATATCAACCCATATCTCATTC
CCTTCTTTATCGGGTTACAGAACCGGTTTACGCAGTTTCGGCTTAGTGAA
ACAAAAGAAATCACCAATCCGTATGCCATGCGTTTATACGAATCCCTGTG
TCAGTATCGTAAGCCGGATGGCTCAGGCATCGTCTCTCTGAAAATCGACT
GGATCATAGAGCGTTACCAGCTGCCTCAAAGTTACCAGCGTATGCCTGAC
TTCCGCCGCCGCTTCCTGCAGGTCTGTGTTAATGAGATCAACAGCAGAAC
TCCAATGCGCCTCTCATACATTGAGAAAAAGAAAGGCCGCCAGACGACTC
ATATCGTATTTTCCTTCCGCGATATCACTTCCATGACGACAGGATAGTCT
GAGGGTTATCTGTCACAGATTTGAGGGTGGTTCGTCACATTTGTTCTGAC
CTACTGAGGGTAATTTGTCACAGTTTTGCTGTTTCCTTCAGCCTGCATGG
ATTTTCTCATACTTTTTGAACTGTAATTTTTAAGGAAGCCAAATTTGAGG
GCAGTTTGTCACAGTTGATTTCCTTCTCTTTCCCTTCGTCATGTGACCTG
ATATCGGGGGTTAGTTCGTCATCATTGATGAGGGTTGATTATCACAGTTT
ATTACTCTGAATTGGCTATCCGCGTGTGTACCTCTACCTGGAGTTTTTCC
CACGGTGGATATTTCTTCTTGCGCTGAGCGTAAGAGCTATCTGACAGAAC
AGTTCTTCTTTGCTTCCTCGCCAGTTCGCTCGCTATGCTCGGTTACACGG
CTGCGGCGAGCGCTAGTGATAATAAGTGACTGAGGTATGTGCTCTTCTTA
TCTCCTTTTGTAGTGTTGCTCTTATTTTAAACAACTTTGCGGTTTTTTGA
TGACTTTGCGATTTTGTTGTTGCTTTGCAGTAAATTGCAAGATTTAATAA
AAAAACGCAAAGCAATGATTAAAGGATGTTCAGAATGAAACTCATGGAAA
CACTTAACCAGTGCATAAACGCTGGTCATGAAATGACGAAGGCTATCGCC
ATTGCACAGTTTAATGATGACAGCCCGGAAGCGAGGAAAATAACCCGGCG
CTGGAGAATAGGTGAAGCAGCGGATTTAGTTGGGGTTTCTTCTCAGGCTA
TCAGAGATGCCGAGAAAGCAGGGCGACTACCGCACCCGGATATGGAAATT
CGAGGACGGGTTGAGCAACGTGTTGGTTATACAATTGAACAAATTAATCA
TATGCGTGATGTGTTTGGTACGCGATTGCGACGTGCTGAAGACGTATTTC
CACCGGTGATCGGGGTTGCTGCCCATAAAGGTGGCGTTTACAAAACCTCA
GTTTCTGTTCATCTTGCTCAGGATCTGGCTCTGAAGGGGCTACGTGTTTT
GCTCGTGGAAGGTAACGACCCCCAGGGAACAGCCTCAATGTATCACGGAT
GGGTACCAGATCTTCATATTCATGCAGAAGACACTCTCCTGCCTTTCTAT
CTTGGGGAAAAGGACGATGTCACTTATGCAATAAAGCCCACTTGCTGGCC
GGGGCTTGACATTATTCCTTCCTGTCTGGCTCTGCACCGTATTGAAACTG
AGTTAATGGGCAAATTTGATGAAGGTAAACTGCCCACCGATCCACACCTG
ATGCTCCGACTGGCCATTGAAACTGTTGCTCATGACTATGATGTCATAGT
TATTGACAGCGCGCCTAACCTGGGTATCGGCACGATTAATGTCGTATGTG
CTGCTGATGTGCTGATTGTTCCCACGCCTGCTGAGTTGTTTGACTACACC
TCCGCACTGCAGTTTTTCGATATGCTTCGTGATCTGCTCAAGAACGTTGA
TCTTAAAGGGTTCGAGCCTGATGTACGTATTTTGCTTACCAAATACAGCA
ATAGTAATGGCTCTCAGTCCCCGTGGATGGAGGAGCAAATTCGGGATGCC
TGGGGAAGCATGGTTCTAAAAAATGTTGTACGTGAAACGGATGAAGTTGG
TAAAGGTCAGATCCGGATGAGAACTGTTTTTGAACAGGCCATTGATCAAC
GCTCTTCAACTGGTGCCTGGAGAAATGCTCTTTCTATTTGGGAACCTGTC
TGCAATGAAATTTTCGATCGTCTGATTAAACCACGCTGGGAGATTAGATA
ATGAAGCGTGCGCCTGTTATTCCAAAACATACGCTCAATACTCAACCGGT
TGAAGATACTTCGTTATCGACACCAGCTGCCCCGATGGTGGATTCGTTAA
TTGCGCGCGTAGGAGTAATGGCTCGCGGTAATGCCATTACTTTGCCTGTA
TGTGGTCGGGATGTGAAGTTTACTCTTGAAGTGCTCCGGGGTGATAGTGT
TGAGAAGACCTCTCGGGTATGGTCAGGTAATGAACGTGACCAGGAGCTGC
TTACTGAGGACGCACTGGATGATCTCATCCCTTCTTTTCTACTGACTGGT
CAACAGACACCGGCGTTCGGTCGAAGAGTATCTGGTGTCATAGAAATTGC
CGATGGGAGTCGCCGTCGTAAAGCTGCTGCACTTACCGAAAGTGATTATC
GTGTTCTGGTTGGCGAGCTGGATGATGAGCAGATGGCTGCATTATCCAGA
TTGGGTAACGATTATCGCCCAACAAGTGCTTATGAACGTGGTCAGCGTTA
TGCAAGCCGATTGCAGAATGAATTTGCTGGAAATATTTCTGCGCTGGCTG
ATGCGGAAAATATTTCACGTAAGATTATTACCCGCTGTATCAACACCGCC
AAATTGCCTAAATCAGTTGTTGCTCTTTTTTCTCACCCCGGTGAACTATC
TGCCCGGTCAGGTGATGCACTTCAAAAAGCCTTTACAGATAAAGAGGAAT
TACTTAAGCAGCAGGCATCTAACCTTCATGAGCAGAAAAAAGCTGGGGTG
ATATTTGAAGCTGAAGAAGTTATCACTCTTTTAACTTCTGTGCTTAAAAC
GTCATCTGCATCAAGAACTAGTTTAAGCTCACGACATCAGTTTGCTCCTG
GAGCGACAGTATTGTATAAGGGCGATAAAATGGTGCTTAACCTGGACAGG
TCTCGTGTTCCAACTGAGTGTATAGAGAAAATTGAGGCCATTCTTAAGGA
ACTTGAAAAGCCAGCACCCTGATGCGACCACGTTTTAGTCTACGTTTATC
TGTCTTTACTTAATGTCCTTTGTTACAGGCCAGAAAGCATAACTGGCCTG
AATATTCTCTCTGGGCCCACTGTTCCACTTGTATCGTCGGTCTGATAATC
AGACTGGGACCACGGTCCCACTCGTATCGTCGGTCTGATTATTAGTCTGG
GACCACGGTCCCACTCGTATCGTCGGTCTGATTATTAGTCTGGGACCACG
GTCCCACTCGTATCGTCGGTCTGATAATCAGACTGGGACCACGGTCCCAC
TCGTATCGTCGGTCTGATTATTAGTCTGGGACCATGGTCCCACTCGTATC
GTCGGTCTGATTATTAGTCTGGGACCACGGTCCCACTCGTATCGTCGGTC
TGATTATTAGTCTGGAACCACGGTCCCACTCGTATCGTCGGTCTGATTAT
TAGTCTGGGACCACGGTCCCACTCGTATCGTCGGTCTGATTATTAGTCTG
GGACCACGATCCCACTCGTGTTGTCGGTCTGATTATCGGTCTGGGACCAC
GGTCCCACTTGTATTGTCGATCAGACTATCAGCGTGAGACTACGATTCCA
TCAATGCCTGTCAAGGGCAAGTATTGACATGTCGTCGTAACCTGTAGAAC
GGAGTAACCTCGGTGTGCGGTTGTATGCCTGCTGTGGATTGCTGCTGTGT
CCTGCTTATCCACAACATTTTGCGCACGGTTATGTGGACAAAATACCTGG
TTACCCAGGCCGTGCCGGCACGTTAACCGGGCTGCATCCGATGCAAGTGT
GTCGCTGTCGACGAGCTCGCGAGCTCGGACATGAGGTTGCCCCGTATTCA
GTGTCGCTGATTTGTATTGTCTGAAGTTGTTTTTACGTTAAGTTGATGCA
GATCAATTAATACGATACCTGCGTCATAATTGATTATTTGACGTGGTTTG
ATGGCCTCCACGCACGTTGTGATATGTAGATGATAATCATTATCACTTTA
CGGGTCCTTTCCGGTGATCCGACAGGTTACGGGGCGGCGACCTCGCGGGT
TTTCGCTATTTATGAAAATTTTCCGGTTTAAGGCGTTTCCGTTCTTCTTC
GTCATAACTTAATGTTTTTATTTAAAATACCCTCTGAAAAGAAAGGAAAC
GACAGGTGCTGAAAGCGAGGCTTTTTGGCCTCTGTCGTTTCCTTTCTCTG
TTTTTGTCCGTGGAATGAACAATGGAAGTCCTGGCGTAATAGCGAAGAGG
CCCGCACCGATCGCCCTTCCCAACAGTTGCGCAGCCTGAATGGCGAATGG
CGCCTGATGCGGTATTTTCTCCTTACGCATCTGTGCGGTATTTCACACCG
CATATGGTGCACTCTCAGTACAATCTGCTCTGATGCCGCATAGTTAAGCC
AGCCCCGACACCCGCCAACACCCGCTGACGCGCCCTGACGGGCTTGTCTG
CTCCCGGCATCCGCTTACAGACAAGCTGTGACCGTCTCCGGGAGCTGCAT
GTGTCAGAGGTTTTCACCGTCATCACCGAAACGCGCGAGACGAAAGGGCC
TCGTGATACGCCTATTTTTATAGGTTAATGTCATGATAATAATGGTTTCT
TAGAC.

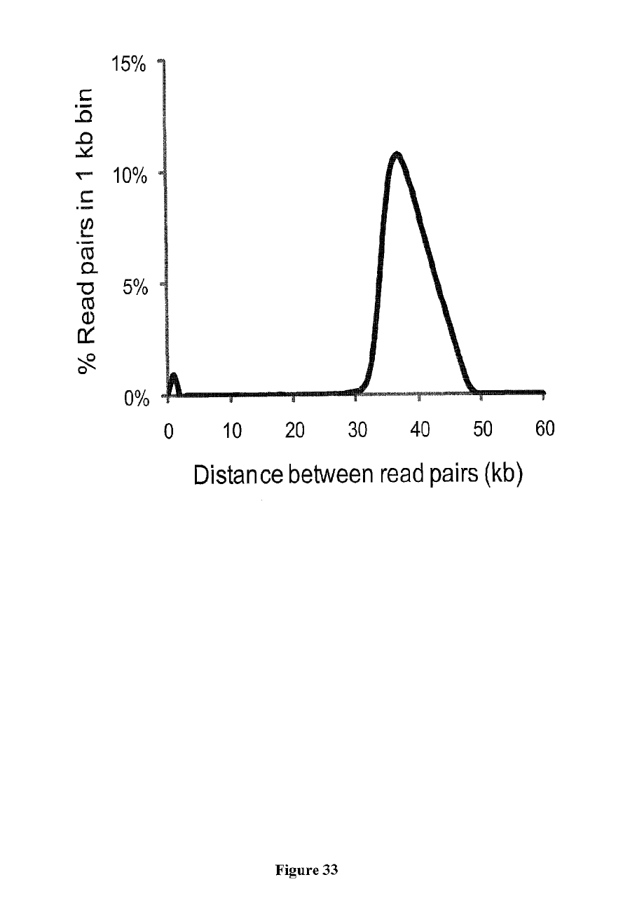

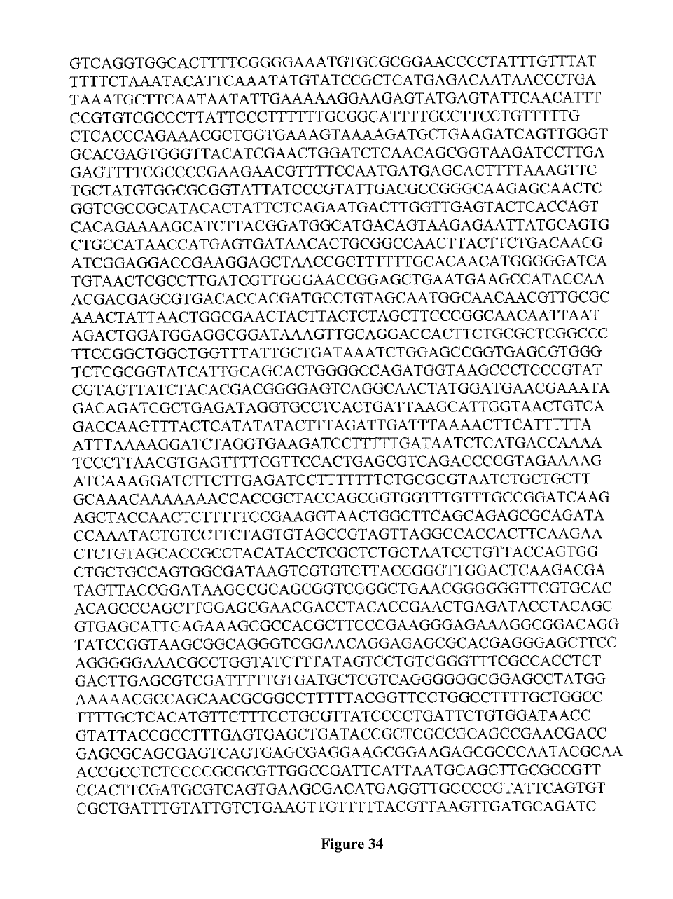

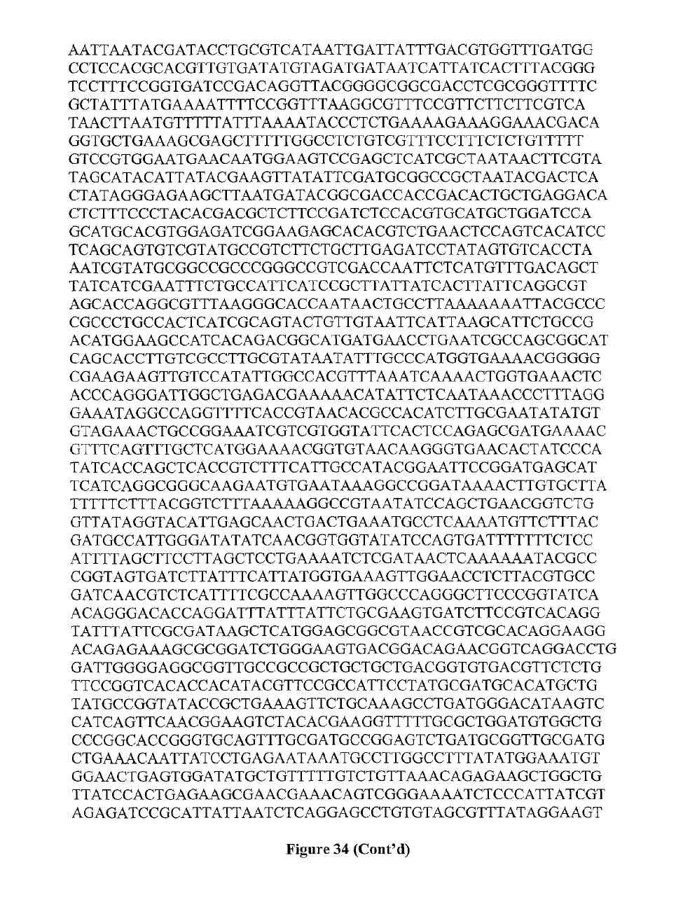

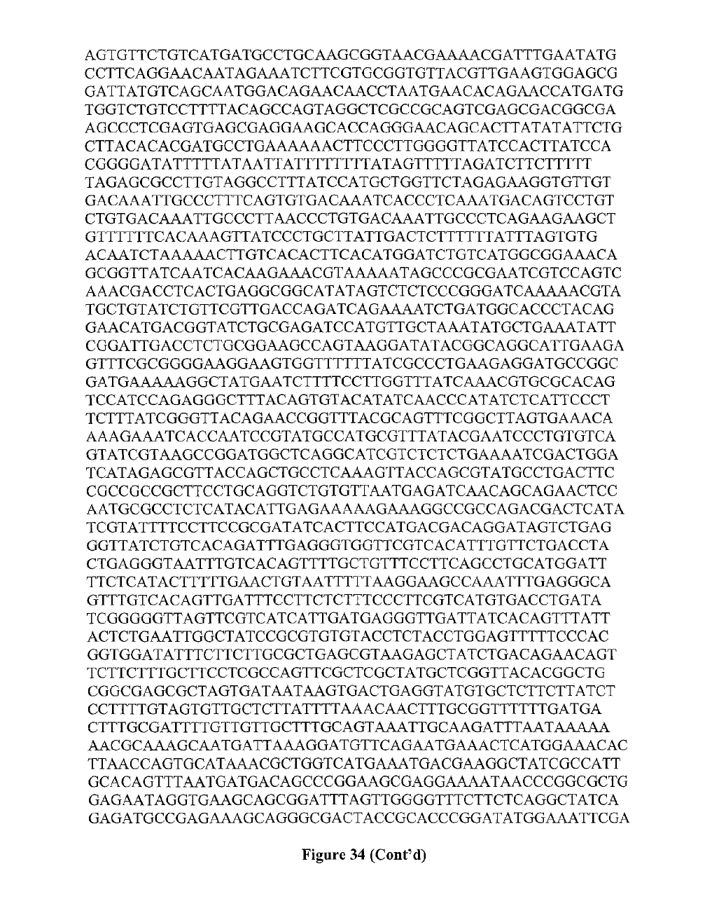

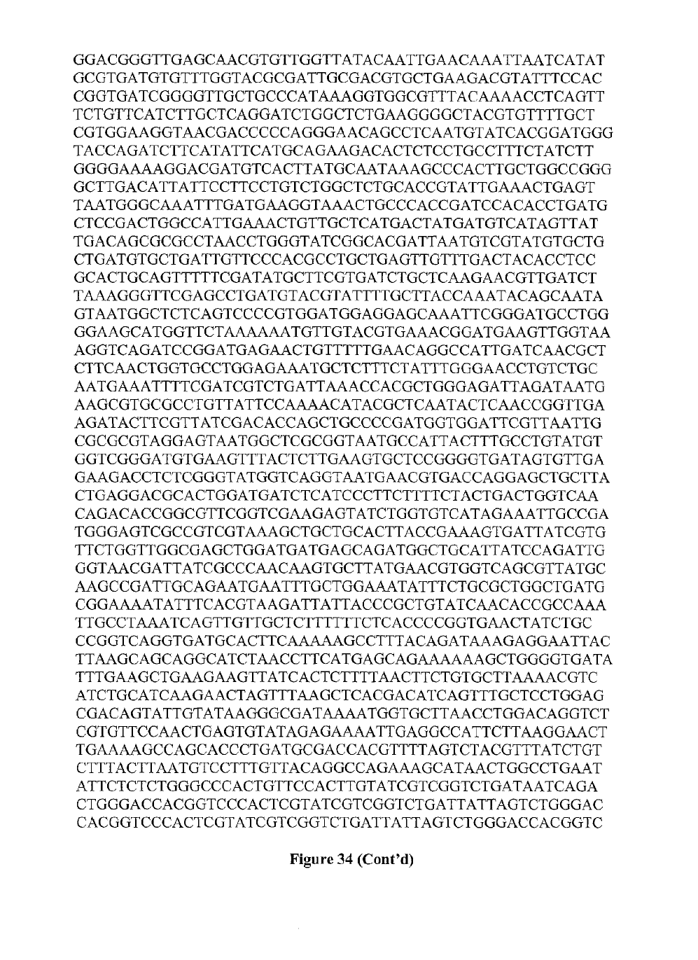

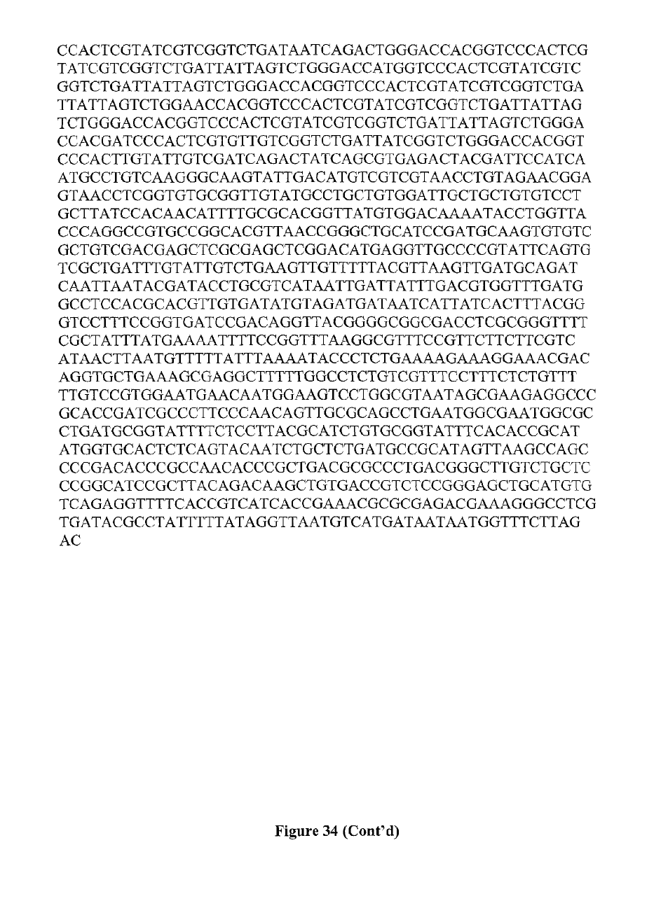

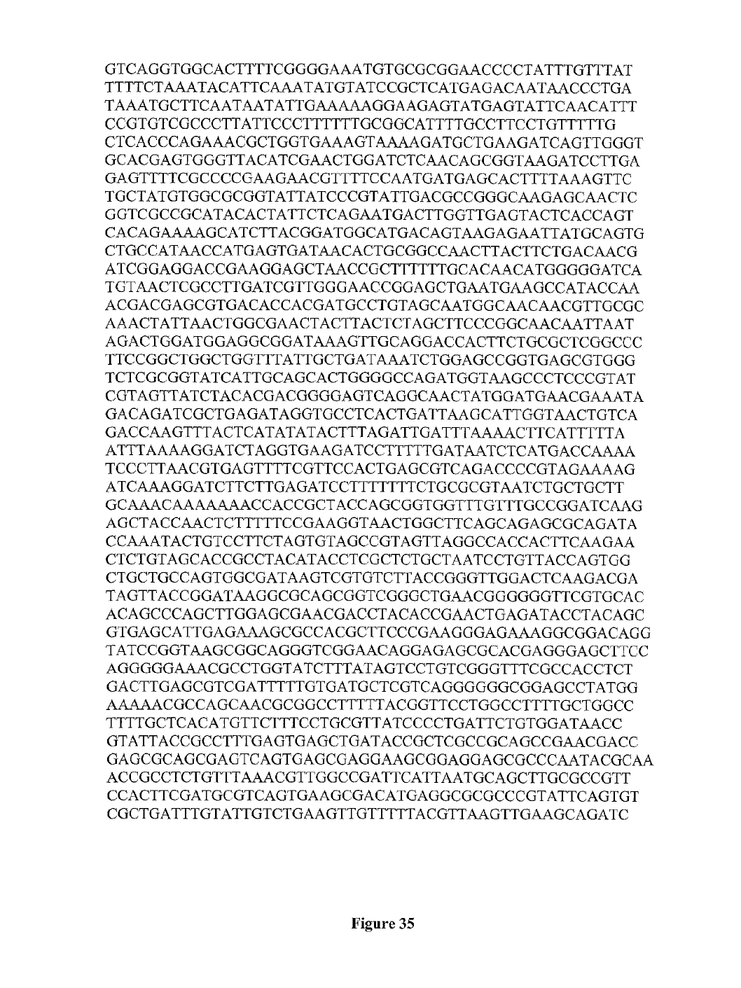

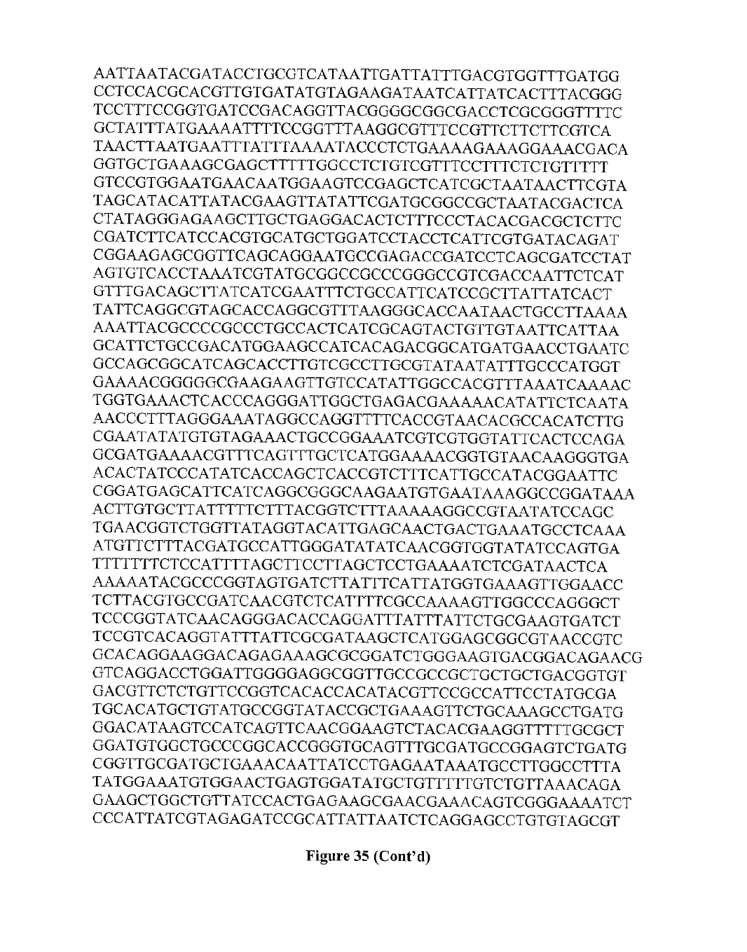

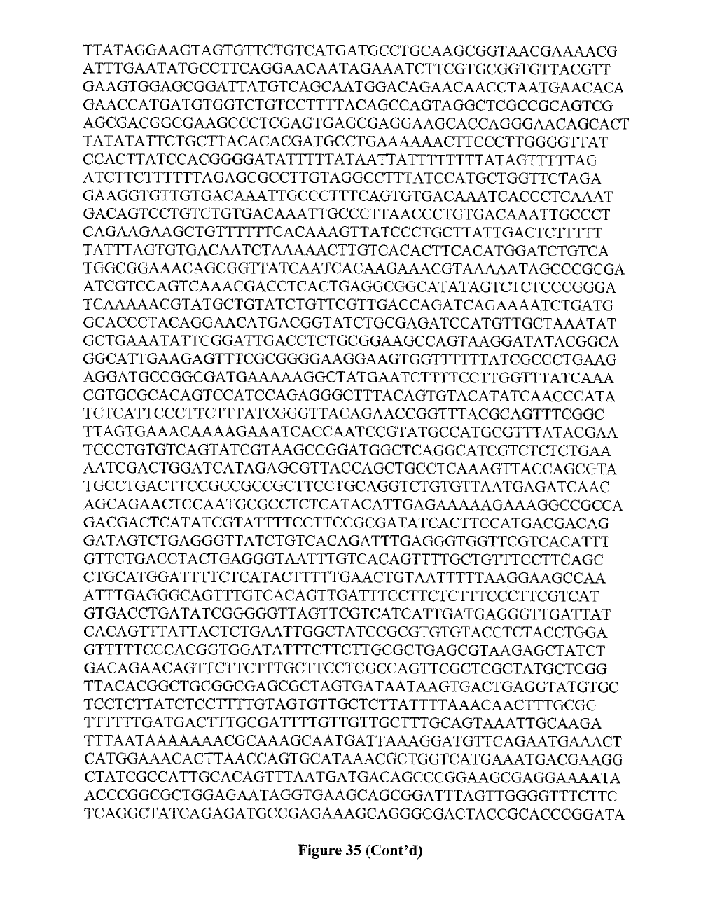

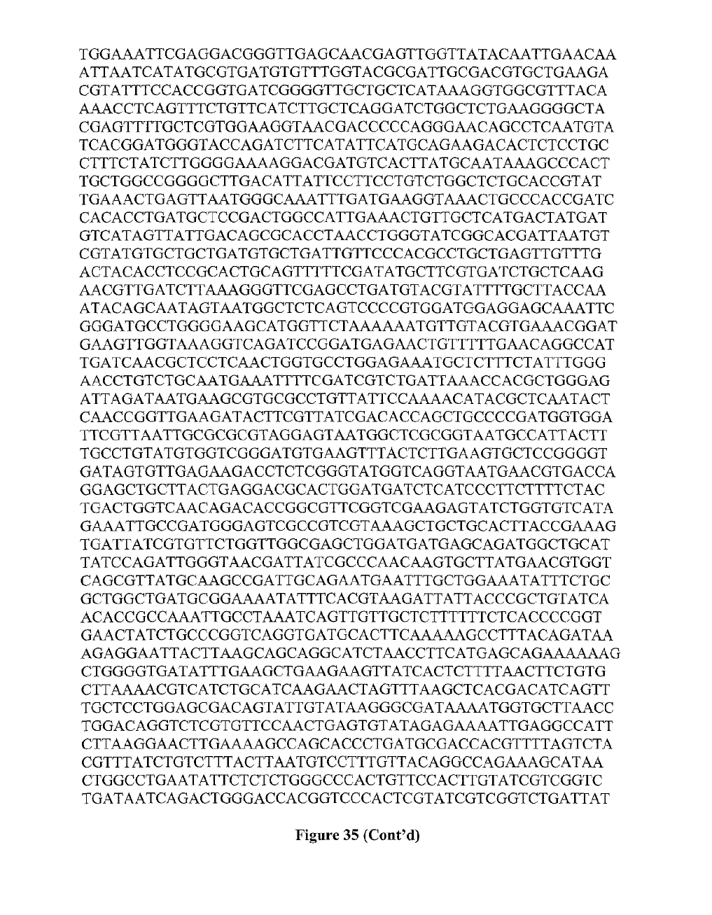

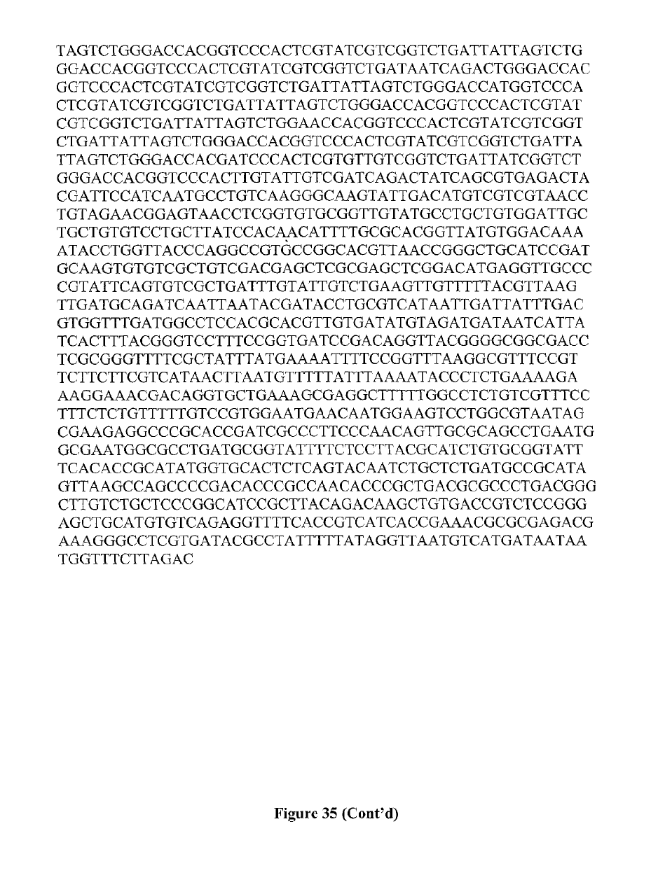

In one embodiment, the library comprises a fosmid library comprising large DNA inserts. In one embodiment, the DNA insert is cleaved into a first and second portions. In one embodiment, the first portion is retained within the fosmid vector and retains the two terminal portions of the large DNA insert. In one embodiment, the second portion comprises the DNA insert without the two original terminal portions. The fosmid vector is then circularized, and inverse PCR amplified, thereby creating an Illumina-sequencing-ready 30-45-kb fosIll jumping library. In one embodiment, the present invention contemplates a method comprising creating a plurality of short sequenceable amplicons (fosIll library) from a cloned fosmid library. In one embodiment, the fosIll library comprises a mouse fosIll library. In one embodiment, the mouse fosIll library comprises a C57B6J mouse library. In one embodiment, the C57B6J mouse library comprises a jump size distribution mean of approximately 38.4 kb. See, FIG. 33. In one embodiment, the C57B6J mouse library comprises a metric selected from the group consisting of a fosmid library size (cfu) of approximately 7.5 million sequences, approximately 18.7 million mapped read pairs, approximately 18.4 million correct 30-50 kb jumps, approximately 0.18 million (˜1%) chimeric jumps, approximately 5.6 million unique correct jumps, and/or an approximate 80-fold genomic coverage.

In typical next-generation high throughput sequencing based upon jumping libraries constructed inverse PCR, if the in vitro cloning process is initiated with approximately 30 μg of starting genomic material then one would expect: i) <10⁸ unique read pairs when using 3 kb jump inserts; ii) <10⁷ unique read pairs when using 10 kb jump inserts; iii) <10⁶ unique read pairs when using a 15 kb jump inserts; or iv) <10⁵ unique read pairs when using 25 kb jump inserts; and even fewer unique productive long-distance read pairs when using jump inserts approaching 40 kb.

In contrast, when using an in vivo cloning techniques, as described herein, approximately 30 μg of starting genomic material yields approximately 10⁶ or more unique long-distance read pairs when using 30-45 kb jump inserts. This suggests an increased yield of ranging between approximately 2-5 orders of magnitude.

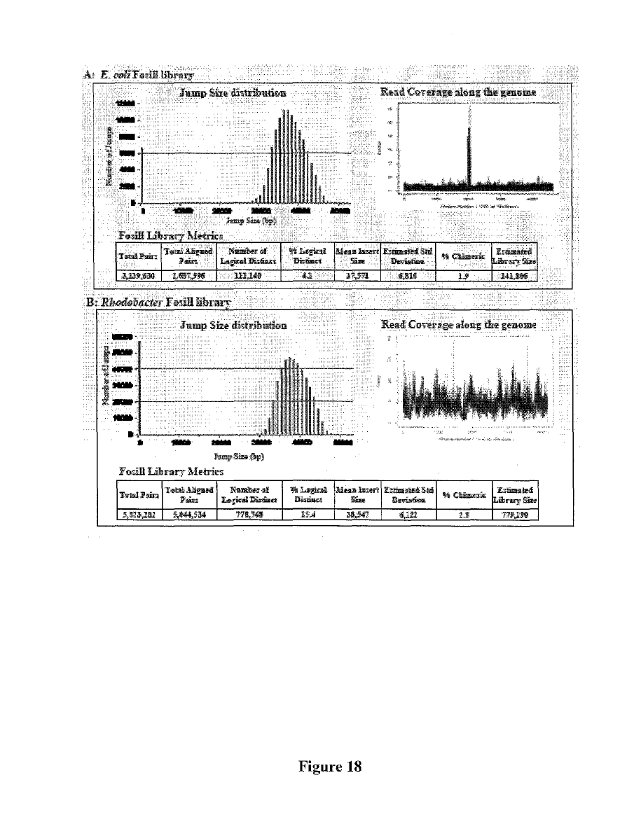

The data presented herein show that the paired-end sequencing reads generated from E. coli and R. sphaeroides fosIll jump libraries had the gap-size distribution expected for fosmid end sequences and a low rate of chimerism (˜2%). See, FIG. 18A & FIG. 18B. E. coli and Rhodobacter fosmid libraries were processed and the data identifies the fosill jump size distribution along with the library's associated metrics and genome coverage. Both libraries show the expected jump size for a fosmid-type library as well as a low number of chimeric molecules. Coverage of the Escherichia coli genome is biased with a large number of sequences around genomic position 2,800,000. This may be due to bias introduced by cloning and propagating an E. coli library within E. coli. The coverage of the R. sphaeroides genome is more even along the genome when propagated within E. coli.

In one embodiment, the present invention contemplates a method comprising sequencing a fosmid clonal library. In one embodiment, the fosmid clonal library comprises an E. coli clone transformed with a lambda bacteriophage, wherein the bacteriophage comprises a fosmid cloning vector carrying an approximate 40 kb DNA insert, wherein the cloning vector comprises a pair of endonuclease sites. In one embodiment, the endonuclease sites are recognized by the nicking enzyme Nb.BbvCI. In one embodiment, the method further comprises nicking the endonuclease sites. In one embodiment, the method further comprises moving the nick by nick translation for several hundred base pairs along the fosmid, thereby creating two nicks within the cloned insert thereby creating two cleavable sites within less than 1 kb of the vector on either side of the vector.

Nick translation has been reported to facilitate methods that are typically used for labeling of DNA. For example, nick translation has also been used in combination with S1 nuclease digestion for removing the bulk of a circularized insert as part of constructing a SOLiD mate-pair jumping library method. McKernan et al., Genome Res. 19:1527-1541 (2009). Although it is not necessary to understand the mechanism of an invention, it is believed that when circularization of a nicked DNA insert is facilitated by dephosphorylation, the nicks may be bidirectionally extended along the DNA insert using a timed nick translation reaction and subsequently cleaved with an S1 nuclease. In the present invention, the nicks are introduced by digestion with Nb.BbvCI.

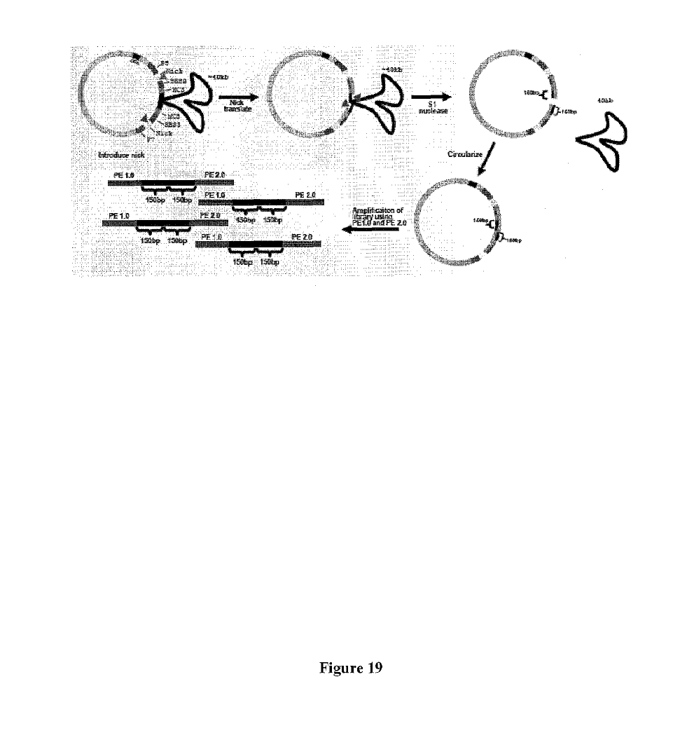

In one embodiment, the method further comprises generating a double-strand break at the site of the translated nicks by cleaving with an S1 nuclease, thereby releasing a first porton and a second portion of the DNA insert fragment. In one embodiment, the cleaving retains the second portion of the DNA insert fragment within the vector, wherein the second portion comprises two terminal vector-adjacent portions that are proximal to the translated nicks. In one embodiment, the second portion is ˜39.5 kb and is released from the vector. In one embodiment, the fosmid vector containing the first portion (i.e., for example, the vector-adjacent insert end portions) on either end is recircularized, followed by inverse PCR of the co-ligated first portion. In one embodiment, the cleaving creates a fosmid vector comprising the two 0.5 kb terminal ends of the DNA insert. In one embodiment, the fosmid vector is used to construct a fosIll jumping library. In one embodiment, the processed fosmid library undergoes an inverse polymerase chain reaction, thereby generating a plurality of linear amplicons (i.e., for example, ˜1M) comprising a plurality of next-generation long-distance read-pairs. In one embodiment, the read pairs are approximately between 50-900 base pairs (i.e., for example, <1 kb), preferably between 75-650 base pairs, more preferably between 100-500 base pairs, and most preferably 400 base pairs. Although it is not necessary to understand the mechanism of an invention, it is believed that the read pairs are best represented by insert terminal ends having approximately equal lengths. In one embodiment, the next-generation read-pairs are sequenced using a sequencing technique selected from the group comprising bridge amplification and/or emulsion amplification. For example, the inverse PCR and the sequencing may use Illumina PE enrichment primer pairs having affinity for the forward and reverse primer recognition sites. See. FIG. 19. In one embodiment, the linear amplicons are compatible with bridge amplification techniques. In one embodiment, the bridge amplification technique comprises an Illumina technique. In one embodiment, the linear amplicons are compatible with emulsion amplification. In one embodiment, the emulsion amplification technique comprises a SOLID technique.

Although it is not necessary to understand the mechanism of an invention, it is believed that the fosIll approach for converting traditional fosmid libraries into jumping libraries compatible with massively parallel paired-end sequencing by Illumina is suitable for generating read pairs that span 30 to 50 kb of genomic distance. It is further believed that the yield and complexity of unique bona fide fosIll jumps is sufficiently high (and the background of short, incorrect spacings and chimeric artifacts sufficiently low) for practical applications such as de novo genome assembly and mapping of structural variations.

To test a pfosIll cloning system, an unamplified fosmid library was constructed starting with 30 μg Schizosaccharomyces pombe, human K562, or mouse C57BL/6J DNA. The library size, as estimated by plating small-scale test transductions on chloramphenicol plates, was 1.4 million colony forming units (efu). A large-scale transduction with the entire library, amplified camR transductants as a pool by overnight liquid culture at 30° C. and prepared fosmid DNA representing the entire library. Approximately 10 μg of fosmid DNA was converted into an Illumina library, sequenced in a 2×101-base paired-end mode on a GAII instrument and aligned the reads to the S. pombe reference genome sequence. See, Table 1.

TABLE 1
Summary statistics for FosIll libraries

| Organism: | S. pombe | Human (K-562) | | Mouse | |
|---|---|---|---|---|---|
| Fosmids (cfu × 10⁻⁶) | 1.4 | 6.6 | | 1.0 | 7.5 |
| FosIll library | S1 | H1 | H2 | M1 | M2 |
| Size selection | 1x prep gel | low range | high range | 1x prep gel | 2x prep gel |
| Unambiguously placed | 18.1 | 33.9 | 9.7 | 21.6 | 18.7 |
| pairs^{a} (×10⁻⁶) | | | | | |
| Correct jumps^{b} (×10⁻⁶) | 17.1 | 30.3 | 9.0 | 20.6 | 18.4 |
| Unique^{c} unambiguously | 1.71 | 6.96 | 4.25 | 1.97 | 5.87 |
| placed pairs (×10⁻⁶) | | | | | |
| Unique^{c} correct jumps | 1.47 | 5.51 | 3.79 | 1.62 | 5.63 |
| (×10⁻⁶) | | | | | |
| Mean correct jump | 37.8 ± 3.4 | 38.6 ± 3.8 | 38.5 ± 3.6 | 38.5 ± 3.5 | 38.4 ± 3.5 |
| length ± s.d. (kb) | | | | | |
| Physical genome coverage | >4,000x | 74x | 51x | 23x | 80x |

| | | | |
|---|---|---|---|
| Total unique correct | 1.47 | 6.93 | 7.25 |
| jumps^{d} (×10⁻⁶) | | | |

^{a}Both reads aligned to a single location in the genome
^{b}Convergent read pairs that aligned 30 to 50 kb apart
^{c}Duplicate read pairs (within each FosIll library) with identical start sites of forward and reverse sequencing reads removed
^{d}All duplicate read pairs (for each organism) with identical start sites of forward and reverse sequencing reads removed

Of 18.1 million unambiguously placed read-pairs, 17.1 million (94%) had the expected spacing (30 to 50 kb) and orientation (convergent). On average, these bona fide Fosmid jumps were 37.8 kb in length. Less than 1% of the aligned read-pairs jumped farther than 100 kb or between chromosomes and thus constituted likely chimeric artifacts. The total number of unique fosmid-size jumps was 1.47 million, about the same as the estimated size of the original fosmid library (1.4 million cfu). At this depth of sequencing (˜12×) we hit essentially all unique 30-50 kb jumps represented in the fosIll library.

A significant fraction (6.3%) of the non-redundant subset of read paired mapped less than 1 kb apart. Manual inspection suggested that a significant fraction thereof represented non-jumps, i.e., single small contiguous genome fragments, and unequal jumps with one of the co-ligated end-fragments very short, possibly caused by lopsided nick translation.

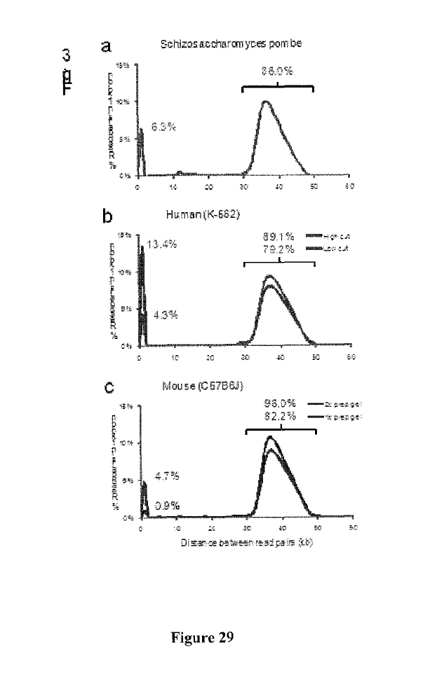

Although it is not necessary to understand the mechanism of an invention, it is believed that the low end of a fosIll-size distribution might be enriched for these undesired side products. The data presented herein describes a fosmid library, ˜6.7 million efu generated from 60 μg of K-562 DNA, a human chronic myelogenous leukemia (CML) cell line. Lozzio et al., “Human chronic myelogenous leukemia cell-line with positive Philadelphia chromosome” Blood 1975, 45(3):321-334. Two size fractions (450-700 bp and 700-900 bp) were excised and sequenced separately. The shorter PCR products contained indeed a higher proportion of inserts spanning <1 kb than the longer ones (13.4% vs. 4.3%). See, FIG. 29B. The number of 30-50 kb jumps that were unique in each sub-library was 5.5 million and 3.8 million, respectively.

Further, two independent fosmid libraries were constructed using Mus musculus DNA. The first library has an estimated size of ˜1 million cfu, and yielded 1.6 million distinct 30-50 kb jumps. Again, about 5% of distinct, non-redundant read-pairs spanned less than 1 kb. For the second library (=7.5 million cfu), short side products were eliminated by re-purifying the size-selected PCR product on a second preparative gel. Only ˜1% of non-redundant read pairs from these doubly size-selected fosIll's aligned less than 1 kb apart.; 96% (5.6 million distinct jumps) spanned 30-50 kb; 2.4% were classified as chimeric. The rate of chimerism for all unambiguously placed read pairs (18.7 million) was about 1%.

To assess the power of fosIll sequencing for detecting gross structural rearrangements, loci in the K-562 genome were searched that were spanned by jumps which were aberrantly spaced or interchromosomal in the human reference genome. For example, 21 distinct rearrangements were identified with 10 or more independent supporting read pairs. See, Table 2.

TABLE 2
Five rearrangements in the K-562 genome
identified by FosIll read pairs

| Supporting | | Rearrange- | Affected | In frame |
|---|---|---|---|---|
| read pairs | Rank^{a} | ment | chromosome(s) | protein fusion |
| 887 | 1 | translocation | 9; 22 | BCR-ABL1 |
| 131 | 2 | inversion | 9 | |
| 130 | 3 | tandem | 6 | BAT3-SLC44A4 |
| | | duplication | | |
| 55 | 7 | deletion | 10 | |
| 18 | 15 | translocation | 9; 22 | NUP214-XKR3 |

^{a}Ranked by number of supporting read pairs

The t(9;22) translocation that gives rise to the BCR-ABL1 fusion protein (ref) was framed by a total of 887 unique “chimeric” read pairs. The large number of BCR-ABL1 hits is consistent with extensive amplification of this locus. Ross et al., “Genomic translocation breakpoint sequences are conserved in BCR-ABL1 cell lines despite the presence of amplification” Cancer Genet Cytogenet 2009, 189(2):138-139. Given the complexity (6.9 million) and average spacing (38.5 kb) of fosIll jumps one would expect ˜90-fold coverage for a single-copy locus.

Two more rearrangements, a tandem duplication on chromosome 6 and a second t(9;22) translocation, were also detected that could plausibly encode in-frame fusion proteins. Of note, chimeric transcripts supporting all three gene fusions (BCR-ABL1, BAT3-SKC44A4 and NUP214-XKR3) have been previously identified in the K-562 transcriptome by RNA-seq. Berger et al., “Integrative analysis of the melanoma transcriptome” Genome Res 2010, 20(4):413-427; and Levin et al., “Targeted next-generation sequencing of a cancer transcriptome enhances detection of sequence variants and novel fusion transcripts” Genome Biol 2009, 10(10):R115. The BCR-ABL1 junction matched the junction sequence reported in the literature. Chissoe et al., “Sequence and analysis of the human ABL gene, the BCR gene, and regions involved in the Philadelphia chromosomal translocation” Genomics 1995, 27(1):67-82; and Shibata et al., “Detection of DNA fusion junctions for BCR-ABL translocations by Anchored ChromPET” Genome Med 2010, 2(9):70.

### III. Shearing And Recircularization (ShaRc) Jumping Libraries

In one embodiment, the present invention contemplates a method to efficiently sequence fosmid libraries on next generation sequencing technologies. In one embodiment, long sequence (i.e., for example, 40 kb) paired end data sets are capable of increasing the scaffold size of de novo genome assemblies. In one embodiment, the paired end data set comprises mouse data. In one embodiment, the paired end data set comprises human data. In one embodiment, the method creates a standard fosmid library that can be processed to generate more than 2 million distinct 40 kb links at a 500× lower cost than conventional capillary sequencing methods.

In some embodiments, a stuffer sequence is attached to the vector sequence before recircularization. Although it is not necessary to understand the mechanism of an invention, it is believed that a sniffer sequence may maintain clone identity and/or improve recircularization efficiency. In one embodiment, a stuffer sequence (i.e., for example, a unique oligo sequence) may uniquely identify a re-circularization junction. In one embodiment, a stuffer sequence may comprise adapter sequences ligated to both ends of the genomic DNA insert, which upon circularization, form a unique sequence that may include, but is not limited to, a barcode, priming sites, or a complementary sequence to increase ligation efficiency (i.e., for example, a sequence comprising sticky ends). In one embodiment, the stuffer comprises internal universal priming sites such that internal genomic DNA sequence amplicons may be obtained. In one embodiment, the stuffer comprises a unique DNA sequence and/or a barcode that enables the identification of a particular sample or genomic DNA fragment as well as facilitates the computational identification of the circularization junction point. In one embodiment, the stuffer comprises universal priming sites and barcodes, thereby allowing for the barcode and a portion of the genomic DNA to be read in sequencing. In one embodiment, the stuffer is constructed upon circularization from adapters ligated to the ends of the genomic DNA molecule, which then form the stuffer upon circularization. These adapters may be designed with complementary sequences such as a single base overhang to facilitate ligation. The barcoded stuffers allow for high levels of multiplexing as multiple samples can be sequenced together reducing costs.

In one embodiment, the present invention contemplates a method for creating in vivo jumping libraries comprising shearing and recircularizing a plasmid (i.e., ShaRe) into short ‘reads’ that are capable of being sequenced and assembled into high quality genomes using massively parallel sequencing technology and a novel assembly algorithm (i e, for example ALLPATHS-LG).

The ShaRe method described herein to support fosmid clone sequencing on an Illumina GAII platform or other high throughput sequencing platforms is an efficient and novel technique to deliver long links to a variety of sequencing projects including mammalian assembly. The data described herein show that the ShaRc method is feasible and comparable in quality to tradition Sanger-capillary based methods, and has one advantage of a significantly lower cost by an estimated factor of 500×. The ShaRc method, coupled with the very high yields of next generation sequencing, has a potential to efficiently deliver very high physical coverage for a range of other applications including, but not limited to, detection of structural variations in cancer and other diseases. In one embodiment, a barcoding method demonstrates multiplexed genomic assembly.

Currently, the sequencing of fosmid paired ends from large 40 kb inserts has been used to provide long range continuity, resolve duplications, and span gaps in de novo whole genome shotgun assembly projects. Further, fosmids have been used to identify structural variation associated with human disease. Traditional methods of fosmid end sequencing based on Sanger labeling and capillary technology are cost, and throughput, prohibitive when using currently available next generation sequencing technologies. Further, next generation sequencing technologies are limited in their ability to produce equivalent data sets due to their shorter read lengths. An alternative known method for preparing fosmids for next generation sequencing relies on a type II restriction digests to create ditags, which inherently limits the length of sequencing reads to short 27 bp tags and therefore does not take advantage of the increasing read lengths of next generation platforms. Kim et al., “Stable propagation of cosmid sized human DNA inserts in an F factor based vector” Nucleic Acids Res. 20(5):1083-1085 (1992).

In one embodiment, the present invention contemplates a method for sequencing a 40 kb fosmid paired end nucleic acid. In one embodiment, the 40 kb fosmid paired end nucleic acid is derived from any standard library of fosmid clones. In one embodiment, the method comprises shearing, re-circularizing, and enriching the fosmid paired end junctions to create a ShaRe fosmid fragment library. In one embodiment, the ShaRc fosmid library is sequenced on a high throughput sequencing platform (i.e., for example, an Illumina GAII or HiSeq platform). Although it is not necessary to understand the mechanism of an invention, it is believed that combining a ShaRc fosmid fragment library with the massively parallel nature of an Illumina GAII sequencing platform allows for recovery of over 1 million distinct 40 kb clones from a single lane of sequencing. It is further believed that the resulting ShaRc fosmid data is equivalent to capillary based fosmid end sequencing but at a greater than 500-fold decrease in cost and a 100-fold decrease in processing time. In one embodiment, the method comprises an improved quality of de novo genome assemblies by sequencing 40 kb ShaRc fosmid paired end data sets with a greater than 30× physical coverage for multiple organisms including Gasterosteus aculeatus (three-spine Stickleback fish), Homo sapiensi (human) or Mus musculus (mouse).

A. ShaRc Fosmid Sample Preparation Method

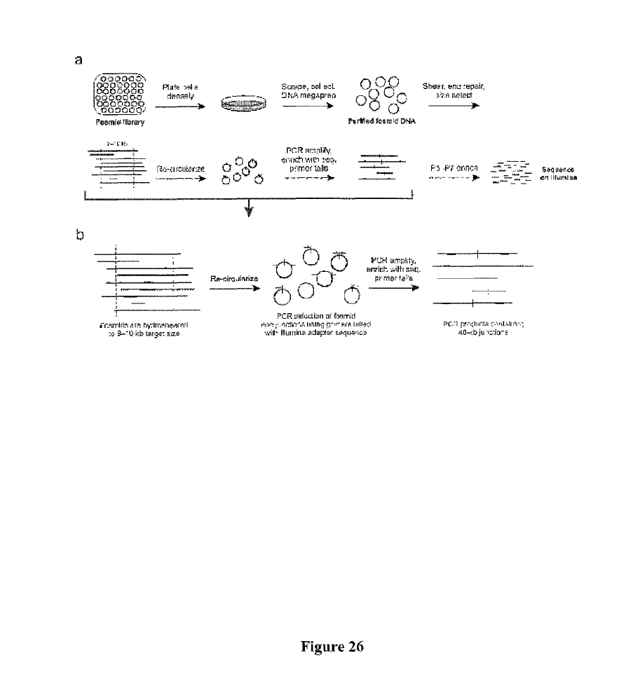

The ShaRc method described herein may be used with any fosmid genomic library. In one embodiment, the fosmid genomic library comprises a pFosCN-1 vector. For illustrative purposes only, the method described here comprises an pEpiFos-5 vector, but any other vector system including, but not limited to, high copy vectors, could be used with a redesign of primers spanning the cloning junction. Briefly, the fosmid genomic clones are plated at high density to the desired depth of coverage, collected, and prepped. In one embodiment, the fosmid genomic clones are sheared (i.e., for example, hydrosheared) to approximately 8-10 kb, thereby creating a subset of fosmid sheared fragments containing the entire fosmid vector of 7.5 kb flanked on either end by approximately 250-1250 bp of genomic insert. In one embodiment, the fosmid 8-10 kb sheared fragments are then re-circularized, thereby creating a ShaRe fosmid fragment. In one embodiment, the ShaRc fosmid fragment comprises a circularized 40 kb junction fosmid fragment. In one embodiment, the method further comprises enriching the circularized 40 kb junction ShaRc fosmid fragments with PCR primers specific to primer binding sites on the fosmid vector sequence, wherein 40 kb ShaRc fosmid junction amplicons are created. See, FIG. 26. In one embodiment, the 40 kb ShaRc fosmid junction amplicons are purified and sequenced using a high throughput sequencing chemistry. In one embodiment, the high throughput sequencing chemistry comprises an Illumina GAII paired read chemistry. Although it is not necessary to understand the mechanism of an invention, it is believed that the paired read chemistry results in ‘a read one’ and ‘a read two’ (i.e., for example, constituting a mate pair read) that maps sequences that are 40 kb apart on a genome. To facilitate the analysis, these reads are trimmed to remove residual vector sequence present in the first 20 bases of each read and the remaining read sequence is aligned to the genome.

In one embodiment, the vector genomic library comprises adapter sequences. In one embodiment, the adapter sequence is a barcode sequence. In one embodiment, the adapter sequence is an enrichment primer binding site. These adapter sequences can contain sequencing primer binding sites for any next-generation technology, enrichment primer binding sites to select for the fragments containing the jump junction, and/or barcode sequence. In one embodiment, the adapter sequences may bind both Illumina sequencing primers (i.e., for example, a primer pair), a universal primer pair for enrichment, and/or a barcode sequence. Although it is not necessary to understand the mechanism of an invention, it is believed that such enriched fragments following fosmid preparation and ShaRc processing produce a ready-to-sequence library with Illumina primer binding sites and barcode sequence immediately prior to the genomic DNA sequence. It is further believed that barcoded adapters may allow for high levels of multiplexing as multiple samples can be combined in a single fosmid library prep and ShaRc processing, thereby greatly reducing the work required for multiple samples. These adapters may be combined with barcoded stuffers in the re-circularization step (infra) allowing for very high combinatorial multiplexing of multiple samples barcoded at the initial genomic DNA step and grouping of samples barcoded at the re-circularization staffer step.

In one embodiment, a pFOS1 cloning vector comprises a pFosCN-1 vector having the nucleotide sequence of:

(SEQ ID NO: 3)
GCGGCCGCAAGGGGTTCGCGTCAGCGGGTGTTGGCGGGTGTCGGGGCTGGCTTA
ACTATGCGGCATCAGAGCAGATTGTACTGAGAGTGCACCATATGCGGTGTGAAA
TACCGCACAGATGCGTAAGGAGAAAATACCGCATCAGGCGCCATTCGCCATTCA
GCTGCGCAACTGTTGGGAAGGGCGATCGGTGCGGGCCTCTTCGCTATTACGCCAG
CTGGCGAAAGGGGGATGTGCTGCAAGGCGATTAAGTTGGGTAACGCCAGGGTTT
TCCCAGTCACGACGTTGTAAAACGACGGCCAGTGAATTGTAATACGACTCACTAT
AGGGCGAATTCACACTCTTTCCCTACACGACGCTCTTCCGATCTCACGTGAGATC
GGAAGAGCGGTTCAGCAGGAATGCCGAGGGATCCTCTAGAGTCGACCTGCAGGC
ATGCAAGCTTGAGTATTCTATAGTCTCACCTAAATAGCTTGGCGTAATCATGGTC
ATAGCTGTTTCCTGTGTGAAATTGTTATCCGCTCACAATTCCACACAACATACGA
GCCGGAAGCATAAAGTGTAAAGCCTGGGGTGCCTAATGAGTGAGCTAACTCACA
TTAATTGCGTTGCGCTCACTGCCCGCTTTCCAGTCGGGAAACCTGTCGTGCCAGC
TGCATTAATGAATCGGCCAACGCGAACCCCTTGCGGCCGCCCGGGCCGTCGACC
AATTCTCATGTTTGACAGCTTATCATCGAATTTCTGCCATTCATCCGCTTATTATC
ACTTATTCAGGCGTAGCAACCAGGCGTTTAAGGGCACCAATAACTGCCTTAAAA
AAATTACGCCCCGCCCTGCCACTCATCGCAGTACTGTTGTAATTCATTAAGCATT
CTGCCGACATGGAAGCCATCACAAACGGCATGATGAACCTGAATCGCCAGCGGC
ATCAGCACCTTGTCGCCTTGCGTATAATATTTGCCCATGGTGAAAACGGGGGCGA
AGAAGTTGTCCATATTGGCCACGTTTAAATCAAAACTGGTGAAACTCACCCAGGG
ATTGGCTGAGACGAAAAACATATTCTCAATAAACCCTTTAGGGAAATAGGCCAG
GTTTTCACCGTAACACGCCACATCTTGCGAATATATGTGTAGAAACTGCCGGAAA
TCGTCGTGGTATTCACTCCAGAGCGATGAAAACGTTTCAGTTTGCTCATGGAAAA
CGGTGTAACAAGGGTGAACACTATCCCATATCACCAGCTCACCGTCTTTCATTGC
CATACGAAATTCCGGATGAGCATTCATCAGGCGGGCAAGAATGTGAATAAAGGC
CGGATAAAACTTGTGCTTATTTTTCTTTACGGTCTTTAAAAAGGCCGTAATATCCA
GCTGAACGGTCTGGTTATAGGTACATTGAGCAACTGACTGAAATGCCTCAAAATG
TTCTTTACGATGCCATTGGGATATATCAACGGTGGTATATCCAGTGATTTTTTTCT
CCATTTTAGCTTCCTTAGCTCCTGAAAATCTCGATAACTCAAAAAATACGCCCGG
TAGTGATCTTATTTCATTATGGTGAAAGTTGGAACCTCTTACGTGCCGATCAACG
TCTCATTTTCGCCAAAAGTTGGCCCAGGGCTTCCCGGTATCAACAGGGACACCAG
GATTTATTTATTCTGCGAAGTGATCTTCCGTCACAGGTATTTATTCGCGATAAGCT
CATGGAGCGGCGTAACCGTCGCACAGGAAGGACAGAGAAAGCGCGGATCTGGG
AAGTGACGGACAGAACGGTCAGGACCTGGATTGGGGAGGCGGTTGCCGCCGCTG
CTGCTGACGGTGTGACGTTCTCTGTTCCGGTCACACCACATACGTTCCGCCATTCC
TATGCGATGCACATGCTGTATGCCGGTATACCGCTGAAAGTTCTGCAAAGCCTGA
TGGGACATAAGTCCATCAGTTCAACGGAAGTCTACACGAAGGTTTTTGCGCTGGA
TGTGGCTGCCCGGCACCGGGTGCAGTTTGCGATGCCGGAGTCTGATGCGGTTGCG
ATGCTGAAACAATTATCCTGAGAATAAATGCCTTGGCCTTTATATGGAAATGTGG
AACTGAGTGGATATGCTGTTTTTGTCTGTTAAACAGAGAAGCTGGCTGTTATCCA
CTGAGAAGCGAACGAAACAGTCGGGAAAATCTCCCATTATCGTAGAGATCCGCA
TTATTAATCTCAGGAGCCTGTGTAGCGTTTATAGGAAGTAGTGTTCTGTCATGAT
GCCTGCAAGCGGTAACGAAAACGATTTGAATATGCCTTCAGGAACAATAGAAAT
CTTCGTGCGGTGTTACGTTGAAGTGGAGCGGATTATGTCAGCAATGGACAGAAC
AACCTAATGAACACAGAACCATGATGTGGTCTGTCCTTTTACAGCCAGTAGTGCT
CGCCGCAGTCGAGCGACAGGGCGAAGCCCTCGAGTGAGCGAGGAAGCACCAGG
GAACAGCACTTATATATTCTGCTTACACACGATGCCTGAAAAAACTTCCCTTGGG
GTTATCCACTTATCCACGGGGATATTTTTATAATTATTTTTTTTATAGTTTTTAGAT
CTTCTTTTTTAGAGCGCCTTGTAGGCCTTTATCCATGCTGGTTCTAGAGAAGGTGT
TGTGACAAATTGCCCTTTCAGTGTGACAAATCACCCTCAAATGACAGTCCTGTCT
GTGACAAATTGCCCTTAACCCTGTGACAAATTGCCCTCAGAAGAAGCTGTTTTTT
CACAAAGTTATCCCTGCTTATTGACTCTTTTTTATTTAGTGTGACAATCTAAAAAC
TTGTCACACTTCACATGGATCTGTCATGGCGGAAACAGCGGTTATCAATCACAAG
AAACGTAAAAATAGCCCGCGAATCGTCCAGTCAAACGACCTCACTGAGGCGGCA
TATAGTCTCTCCCGGGATCAAAAACGTATGCTGTATCTGTTCGTTGACCAGATCA
GAAAATCTGATGGCACCCTACAGGAACATGACGGTATCTGCGAGATCCATGTTG
CTAAATATGCTGAAATATTCGGATTGACCTCTGCGGAAGCCAGTAAGGATATACG
GCAGGCATTGAAGAGTTTCGCGGGGAAGGAAGTGGTTTTTTATCGCCCTGAAGA
GGATGCCGGCGATGAAAAAGGCTATGAATCTTTTCCTTGGTTTATCAAACGTGCG
CACAGTCCATCCAGAGGGCTTTACAGTGTACATATCAACCCATATCTCATTCCCT
TCTTTATCGGGTTACAGAACCGGTTTACGCAGTTTCGGCTTAGTGAAACAAAAGA
AATCACCAATCCGTATGCCATGCGTTTATACGAATCCCTGTGTCAGTATCGTAAG
CCGGATGGCTCAGGCATCGTCTCTCTGAAAATCGACTGGATCATAGAGCGTTACC
AGCTGCCTCAAAGTTACCAGCGTATGCCTGACTTCCGCCGCCGCTTCCTGCAGGT
CTGTGTTAATGAGATCAACAGCAGAACTCCAATGCGCCTCTCATACATTGAGAAA
AAGAAAGGCCGCCAGACGACTCATATCGTATTTTCCTTCCGCGATATCACTTCCA
TGACGACAGGATAGTCTGAGGGTTATCTGTCACAGATTTGAGGGTGGTTCGTCAC
ATTTGTTCTGACCTACTGAGGGTAATTTGTCACAGTTTTGCTGTTTCCTTCAGCCT
GCATGGATTTTCTCATACTTTTTGAACTGTAATTTTTAAGGAAGCCAAATTTGAGG
GCAGTTTGTCACAGTTGATTTCCTTCTCTTTCCCTTCGTCATGTGACCTGATATCG
GGGGTTAGTTCGTCATCATTGATGAGGGTTGATTATCACAGTTTATTACTCTGAAT
TGGCTATCCGCGTGTGTACCTCTACCTGGAGTTTTTCCCACGGTGGATATTTCTTC
TTGCGCTGAGCGTAAGAGCTATCTGACAGAACAGTTCTTCTTTGCTTCCTCGCCA
GTTCGCTCGCTATGCTCGGTTACACGGCTGCGGCGAGCGCTAGTGATAATAAGTG
ACTGAGGTATGTGCTCTTCTTATCTCCTTTTGTAGTGTTGCTCTTATTTTAAACAA
CTTTGCGGTTTTTTGATGACTTTGCGATTTTGTTGTTGCTTTGCAGTAAATTGCAA
GATTTAATAAAAAAACGCAAAGCAATGATTAAAGGATGTTCAGAATGAAACTCA
TGGAAACACTTAACCAGTGCATAAACGCTGGTCATGAAATGACGAAGGCTATCG
CCATTGCACAGTTTAATGATGACAGCCCGGAAGCGAGGAAAATAACCCGGCGCT
GGAGAATAGGTGAAGCAGCGGATTTAGTTGGGGTTTCTTCTCAGGCTATCAGAG
ATGCCGAGAAAGCAGGGCGACTACCGCACCCGGATATGGAAATTCGAGGACGGG
TTGAGCAACGTGTTGGTTATACAATTGAACAAATTAATCATATGCGTGATGTGTT
TGGTACGCGATTGCGACGTGCTGAAGACGTATTTCCACCGGTGATCGGGGTTGCT
GCCCATAAAGGTGGCGTTTACAAAACCTCAGTTTCTGTTCATCTTGCTCAGGATC
TGGCTCTGAAGGGGCTACGTGTTTTGCTCGTGGAAGGTAACGACCCCCAGGGAA
CAGCCTCAATGTATCACGGATGGGTACCAGATCTTCATATTCATGCAGAAGACAC
TCTCCTGCCTTTCTATCTTGGGGAAAAGGACGATGTCACTTATGCAATAAAGCCC
ACTTGCTGGCCGGGGCTTGACATTATTCCTTCCTGTCTGGCTCTGCACCGTATTGA
AACTGAGTTAATGGGCAAATTTGATGAAGGTAAACTGCCCACCGATCCACACCT
GATGCTCCGACTGGCCATTGAAACTGTTGCTCATGACTATGATGTCATAGTTATT
GACAGCGCGCCTAACCTGGGTATCGGCACGATTAATGTCGTATGTGCTGCTGATG
TGCTGATTGTTCCCACGCCTGCTGAGTTGTTTGACTACACCTCCGCACTGCAGTTT
TTCGATATGCTTCGTGATCTGCTCAAGAACGTTGATCTTAAAGGGTTCGAGCCTG
ATGTACGTATTTTGCTTACCAAATACAGCAATAGTAATGGCTCTCAGTCCCCGTG
GATGGAGGAGCAAATTCGGGATGCCTGGGGAAGCATGGTTCTAAAAAATGTTGT
ACGTGAAACGGATGAAGTTGGTAAAGGTCAGATCCGGATGAGAACTGTTTTTGA
ACAGGCCATTGATCAACGCTCTTCAACTGGTGCCTGGAGAAATGCTCTTTCTATT
TGGGAACCTGTCTGCAATGAAATTTTCGATCGTCTGATTAAACCACGCTGGGAGA
TTAGATAATGAAGCGTGCGCCTGTTATTCCAAAACATACGCTCAATACTCAACCG
GTTGAAGATACTTCGTTATCGACACCAGCTGCCCCGATGGTGGATTCGTTAATTG
CGCGCGTAGGAGTAATGGCTCGCGGTAATGCCATTACTTTGCCTGTATGTGGTCG
GGATGTGAAGTTTACTCTTGAAGTGCTCCGGGGTGATAGTGTTGAGAAGACCTCT
CGGGTATGGTCAGGTAATGAACGTGACCAGGAGCTGCTTACTGAGGACGCACTG
GATGATCTCATCCCTTCTTTTCTACTGACTGGTCAACAGACACCGGCGTTCGGTCG
AAGAGTATCTGGTGTCATAGAAATTGCCGATGGGAGTCGCCGTCGTAAAGCTGCT
GCACTTACCGAAAGTGATTATCGTGTTCTGGTTGGCGAGCTGGATGATGAGCAGA
TGGCTGCATTATCCAGATTGGGTAACGATTATCGCCCAACAAGTGCTTATGAACG
TGGTCAGCGTTATGCAAGCCGATTGCAGAATGAATTTGCTGGAAATATTTCTGCG
CTGGCTGATGCGGAAAATATTTCACGTAAGATTATTACCCGCTGTATCAACACCG
CCAAATTGCCTAAATCAGTTGTTGCTCTTTTTTCTCACCCCGGTGAACTATCTGCC
CGGTCAGGTGATGCACTTCAAAAAGCCTTTACAGATAAAGAGGAATTACTTAAG
CAGCAGGCATCTAACCTTCATGAGCAGAAAAAAGCTGGGGTGATATTTGAAGCT
GAAGAAGTTATCACTCTTTTAACTTCTGTGCTTAAAACGTCATCTGCATCAAGAA
CTAGTTTAAGCTCACGACATCAGTTTGCTCCTGGAGCGACAGTATTGTATAAGGG
CGATAAAATGGTGCT′TAACCTGGACAGGTCTCGTGTTCCAACTGAGTGTATAGAG
AAAATTGAGGCCATTCTTAAGGAACTTGAAAAGCCAGCACCCTGATGCGACCAC
GTTTTAGTCTACGTTTATCTGTCTTTACTTAATGTCCTTTGTTACAGGCCAGAAAG
CATAACTGGCCTGAATATTCTCTCTGGGCCCACTGTTCCACTTGTATCGTCGGTCT
GATAATCAGACTGGGACCACGGTCCCACTCGTATCGTCGGTCTGATTATTAGTCT
GGGACCACGGTCCCACTCGTATCGTCGGTCTGATTATTAGTCTGGGACCACGGTC
CCACTCGTATCGTCGGTCTGATAATCAGACTGGGACCACGGTCCCACTCGTATCG
TCGGTCTGATTATTAGTCTGGGACCATGGTCCCACTCGTATCGTCGGTCTGATTAT
TAGTCTGGGACCACGGTCCCACTCGTATCGTCGGTCTGATTATTAGTCTGGAACC
ACGGTCCCACTCGTATCGTCGGTCTGATTATTAGTCTGGGACCACGGTCCCACTC
GTATCGTCGGTCTGATTATTAGTCTGGGACCACGATCCCACTCGTGTTGTCGGTCT
GATTATCGGTCTGGGACCACGGTCCCACTTGTATTGTCGATCAGACTATCAGCGT
GAGACTACGATTCCATCAATGCCTGTCAAGGGCAAGTATTGACATGTCGTCGTAA
CCTGTAGAACGGAGTAACCTCGGTGTGCGGTTGTATGCCTGCTGTGGATTGCTGC
TGTGTCCTGCTTATCCACAACATTTTGCGCACGGTTATGTGGACAAAATACCTGG
TTACCCAGGCCGTGCCGGCACGTTAACCGGGCTGCATCCGATGCAAGTGTGTCGC
TGTCGACGAGCTCGCGAGCTCGGACATGAGGTTGCCCCGTATTCAGTGTCGCTGA
TTTGTATTGTCTGAAGTTGTTTTTACGTTAAGTTGATGCAGATCAATTAATACGAT
ACCTGCGTCATAATTGATTATTTGACGTGGTTTGATGGCCTCCACGCACGTTGTG
ATATGTAGATGATAATCATTATCACTTTACGGGTCCTTTCCGGTGATCCGACAGG
TTACGGGGCGGCGACCTCGCGGGTTTTCGCTATTTATGAAAATTTTCCGGTTTAA
GGCGTTTCCGTTCTTCTTCGTCATAACTTAATGTTTTTATTTAAAATACCCTCTGA
AAAGAAAGGAAACGACAGGTGCTGAAAGCGAGCTTTTTGGCCTCTGTCGTTTCCT
TTCTCTGTTTTTGTCCGTGGAATGAACAATGGAAGTCCGAGCTCATCGCTAATAA
CTTCGTATAGCATACATTATACGAAGTTATATTCGA.

In one embodiment, the cloning site is also flanked by an 8-base variable molecular barcode allowing for pooling of vector constructs, along with SBS3 and SBS12, the Illumina sequencing primer binding sites for the forward and reverse indexed read, respectively, thereby creating a pFosCN-2 cloning vector. In one embodiment, the modified pFOS1 cloning vector comprises a pFosCN-2 vector having the nucleotide sequence of, where “NNNNNNNN” represents the variable 8-base molecular barcode, creating the following pFosCN-2 vector sequence:

(SEQ ID NO: 4)
GCGGCCGCAAGGGGTTCGCGTCAGCGGGTGTTGGCGGGTGTCGGGGCTGGCTTA
ACTATGCGGCATCAGAGCAGATTGTACTGAGAGTGCACCATATGCGGTGTGAAA
TACCGCACAGATGCGTAAGGAGAAAATACCGCATCAGGCGCCATTCGCCATTCA
GCTGCGCAACTGTTGGGAAGGGCGATCGGTGCGGGCCTCTTCGCTATTACGCCAG
CTGGCGAAAGGGGGATGTGCTGCAAGGCGATTAAGTTGGGTAACGCCAGGGTTT
TCCCAGTCACGACGTTGTAAAACGACGGCCAGTGAATTGTAATACGACTCACTAT
AGGGCGAATTCACACTCTTTCCCTACACGACGCTCTTCCGATCTCACGTGAGATC
GGAAGAGCACACGTCTGAACTCCAGTCACNNNNNNNNGGATCCTCTAGAGTCGAC
CTGCAGGCATGCAAGCTTGAGTATTCTATAGTCTCACCTAAATAGCTTGGCGTAA
TCATGGTCATAGCTGTTTCCTGTGTGAAATTGTTATCCGCTCACAATTCCACACAA
CATACGAGCCGGAAGCATAAAGTGTAAAGCCTGGGGTGCCTAATGAGTGAGCTA
ACTCACATTAATTGCGTTGCGCTCACTGCCCGCTTTCCAGTCGGGAAACCTGTCG
TGCCAGCTGCATTAATGAATCGGCCAACGCGAACCCCTTGCGGCCGCCCGGGCC
GTCGACCAATTCTCATGTTTGACAGCTTATCATCGAATTTCTGCCATTCATCCGCT
TATTATCACTTATTCAGGCGTAGCAACCAGGCGTTTAAGGGCACCAATAACTGCC
TTAAAAAAATTACGCCCCGCCCTGCCACTCATCGCAGTACTGTTGTAATTCATTA
AGCATTCTGCCGACATGGAAGCCATCACAAACGGCATGATGAACCTGAATCGCC
AGCGGCATCAGCACCTTGTCGCCTTGCGTATAATATTTGCCCATGGTGAAAACGG
GGGCGAAGAAGTTGTCCATATTGGCCACGTTTAAATCAAAACTGGTGAAACTCA
CCCAGGGATTGGCTGAGACGAAAAACATATTCTCAATAAACCCTTTAGGGAAAT
AGGCCAGGTTTTCACCGTAACACGCCACATCTTGCGAATATATGTGTAGAAACTG
CCGGAAATCGTCGTGGTATTCACTCCAGAGCGATGAAAACGTTTCAGTTTGCTCA
TGGAAAACGGTGTAACAAGGGTGAACACTATCCCATATCACCAGCTCACCGTCTT
TCATTGCCATACGAAATTCCGGATGAGCATTCATCAGGCGGGCAAGAATGTGAA
TAAAGGCCGGATAAAACTTGTGCTTATTTTTCTTTACGGTCTTTAAAAAGGCCGT
AATATCCAGCTGAACGGTCTGGTTATAGGTACATTGAGCAACTGACTGAAATGCC
TCAAAATGTTCTTTACGATGCCATTGGGATATATCAACGGTGGTATATCCAGTGA
TTTTTTTCTCCATTTTAGCTTCCTTAGCTCCTGAAAATCTCGATAACTCAAAAAAT
ACGCCCGGTAGTGATCTTATTTCATTATGGTGAAAGTTGGAACCTCTTACGTGCC
GATCAACGTCTCATTTTCGCCAAAAGTTGGCCCAGGGCTTCCCGGTATCAACAGG
GACACCAGGATTTATTTATTCTGCGAAGTGATCTTCCGTCACAGGTATTTATTCGC
GATAAGCTCATGGAGCGGCGTAACCGTCGCACAGGAAGGACAGAGAAAGCGCG
GATCTGGGAAGTGACGGACAGAACGGTCAGGACCTGGATTGGGGAGGCGGTTGC
CGCCGCTGCTGCTGACGGTGTGACGTTCTCTGTTCCGGTCACACCACATACGTTC
CGCCATTCCTATGCGATGCACATGCTGTATGCCGGTATACCGCTGAAAGTTCTGC
AAAGCCTGATGGGACATAAGTCCATCAGTTCAACGGAAGTCTACACGAAGGTTT
TTGCGCTGGATGTGGCTGCCCGGCACCGGGTGCAGTTTGCGATGCCGGAGTCTGA
TGCGGTTGCGATGCTGAAACAATTATCCTGAGAATAAATGCCTTGGCCTTTATAT
GGAAATGTGGAACTGAGTGGATATGCTGTTTTTGTCTGTTAAACAGAGAAGCTGG
CTGTTATCCACTGAGAAGCGAACGAAACAGTCGGGAAAATCTCCCATTATCGTA
GAGATCCGCATTATTAATCTCAGGAGCCTGTGTAGCGTTTATAGGAAGTAGTGTT
CTGTCATGATGCCTGCAAGCGGTAACGAAAACGATTTGAATATGCCTTCAGGAAC
AATAGAAATCTTCGTGCGGTGTTACGTTGAAGTGGAGCGGATTATGTCAGCAATG
GACAGAACAACCTAATGAACACAGAACCATGATGTGGTCTGTCCTTTTACAGCCA
GTAGTGCTCGCCGCAGTCGAGCGACAGGGCGAAGCCCTCGAGTGAGCGAGGAAG
CACCAGGGAACAGCACTTATATATTCTGCTTACACACGATGCCTGAAAAAACTTC
CCTTGGGGTTATCCACTTATCCACGGGGATATTTTTATAATTATTTTTTTTATAGTT
TTTAGATCTTCTTTTTTAGAGCGCCTTGTAGGCCTTTATCCATGCTGGTTCTAGAG
AAGGTGTTGTGACAAATTGCCCTTTCAGTGTGACAAATCACCCTCAAATGACAGT
CCTGTCTGTGACAAATTGCCCTTAACCCTGTGACAAATTGCCCTCAGAAGAAGCT
GTTTTTTCACAAAGTTATCCCTGCTTATTGACTCTTTTTTATTTAGTGTGACAATCT
AAAAACTTGTCACACTTCACATGGATCTGTCATGGCGGAAACAGCGGTTATCAAT
CACAAGAAACGTAAAAATAGCCCGCGAATCGTCCAGTCAAACGACCTCACTGAG
GCGGCATATAGTCTCTCCCGGGATCAAAAACGTATGCTGTATCTGTTCGTTGACC
AGATCAGAAAATCTGATGGCACCCTACAGGAACATGACGGTATCTGCGAGATCC
ATGTTGCTAAATATGCTGAAATATTCGGATTGACCTCTGCGGAAGCCAGTAAGGA
TATACGGCAGGCATTGAAGAGTTTCGCGGGGAAGGAAGTGGTTTTTTATCGCCCT
GAAGAGGATGCCGGCGATGAAAAAGGCTATGAATCTTTTCCTTGGTTTATCAAAC
GTGCGCACAGTCCATCCAGAGGGCTTTACAGTGTACATATCAACCCATATCTCAT
TCCCTTCTTTATCGGGTTACAGAACCGG′TTTACGCAGTTTCGGCTTAGTGAAACA
AAAGAAATCACCAATCCGTATGCCATGCGTTTATACGAATCCCTGTGTCAGTATC
GTAAGCCGGATGGCTCAGGCATCGTCTCTCTGAAAATCGACTGGATCATAGAGC
GTTACCAGCTGCCTCAAAGTTACCAGCGTATGCCTGACTTCCGCCGCCGCTTCCT
GCAGGTCTGTGTTAATGAGATCAACAGCAGAACTCCAATGCGCCTCTCATACATT
GAGAAAAAGAAAGGCCGCCAGACGACTCATATCGTATTTTCCTTCCGCGATATCA
CTTCCATGACGACAGGATAGTCTGAGGGTTATCTGTCACAGATTTGAGGGTGGTT
CGTCACATTTGTTCTGACCTACTGAGGGTAATTTGTCACAGTTTTGCTGTTTCCTT
CAGCCTGCATGGATTTTCTCATACTTTTTGAACTGTAATTTTTAAGGAAGCCAAAT
TTGAGGGCAGTTTGTCACAGTTGATTTCCTTCTCTTTCCCTTCGTCATGTGACCTG
ATATCGGGGGTTAGTTCGTCATCATTGATGAGGGTTGATTATCACAGTTTATTACT
CTGAATTGGCTATCCGCGTGTGTACCTCTACCTGGAGTTTTTCCCACGGTGGATAT
TTCTTCTTGCGCTGAGCGTAAGAGCTATCTGACAGAACAGTTCTTCTTTGCTTCCT
CGCCAGTTCGCTCGCTATGCTCGGTTACACGGCTGCGGCGAGCGCTAGTGATAAT
AAGTGACTGAGGTATGTGCTCTTCTTATCTCCTTTTGTAGTGTTGCTCTTATTTTA
AACAACTTTGCGGTTTTTTGATGACTTTGCGATTTTGTTGTTGCTTTGCAGTAAAT
TGCAAGATTTAATAAAAAAACGCAAAGCAATGATTAAAGGATGTTCAGAATGAA
ACTCATGGAAACACTTAACCAGTGCATAAACGCTGGTCATGAAATGACGAAGGC
TATCGCCATTGCACAGTTTAATGATGACAGCCCGGAAGCGAGGAAAATAACCCG
GCGCTGGAGAATAGGTGAAGCAGCGGATTTAGTTGGGGTTTCTTCTCAGGCTATC
AGAGATGCCGAGAAAGCAGGGCGACTACCGCACCCGGATATGGAAATTCGAGG
ACGGGTTGAGCAACGTGTTGGTTATACAATTGAACAAATTAATCATATGCGTGAT
GTGTTTGGTACGCGATTGCGACGTGCTGAAGACGTATTTCCACCGGTGATCGGGG
TTGCTGCCCATAAAGGTGGCGTTTACAAAACCTCAGTTTCTGTTCATCTTGCTCAG
GATCTGGCTCTGAAGGGGCTACGTGTTTTGCTCGTGGAAGGTAACGACCCCCAGG
GAACAGCCTCAATGTATCACGGATGGGTACCAGATCTTCATATTCATGCAGAAGA
CACTCTCCTGCCTTTCTATCTTGGGGAAAAGGACGATGTCACTTATGCAATAAAG
CCCACTTGCTGGCCGGGGCTTGACATTATTCCTTCCTGTCTGGCTCTGCACCGTAT
TGAAACTGAGTTAATGGGCAAATTTGATGAAGGTAAACTGCCCACCGATCCACA
CCTGATGCTCCGACTGGCCATTGAAACTGTTGCTCATGACTATGATGTCATAGTT
ATTGACAGCGCGCCTAACCTGGGTATCGGCACGATTAATGTCGTATGTGCTGCTG
ATGTGCTGATTGTTCCCACGCCTGCTGAGTTGTTTGACTACACCTCCGCACTGCAG
TTTTTCGATATGCTTCGTGATCTGCTCAAGAACGTTGATCTTAAAGGGTTCGAGCC
TGATGTACGTATTTTGCTTACCAAATACAGCAATAGTAATGGCTCTCAGTCCCCG
TGGATGGAGGAGCAAATTCGGGATGCCTGGGGAAGCATGGTTCTAAAAAATGTT
GTACGTGAAACGGATGAAGTTGGTAAAGGTCAGATCCGGATGAGAACTGTTTTT
GAACAGGCCATTGATCAACGCTCTTCAACTGGTGCCTGGAGAAATGCTCTTTCTA
TTTGGGAACCTGTCTGCAATGAAATTTTCGATCGTCTGATTAAACCACGCTGGGA
GATTAGATAATGAAGCGTGCGCCTGTTATTCCAAAACATACGCTCAATACTCAAC
CGGTTGAAGATACTTCGTTATCGACACCAGCTGCCCCGATGGTGGATTCGTTAAT
TGCGCGCGTAGGAGTAATGGCTCGCGGTAATGCCATTACTTTGCCTGTATGTGGT
CGGGATGTGAAGTTTACTCTTGAAGTGCTCCGGGGTGATAGTGTTGAGAAGACCT
CTCGGGTATGGTCAGGTAATGAACGTGACCAGGAGCTGCTTACTGAGGACGCAC
TGGATGATCTCATCCCTTCTTTTCTACTGACTGGTCAACAGACACCGGCGTTCGGT
CGAAGAGTATCTGGTGTCATAGAAATTGCCGATGGGAGTCGCCGTCGTAAAGCT
GCTGCACTTACCGAAAGTGATTATCGTGTTCTGGTTGGCGAGCTGGATGATGAGC
AGATGGCTGCATTATCCAGATTGGGTAACGATTATCGCCCAACAAGTGCTTATGA
ACGTGGTCAGCGTTATGCAAGCCGATTGCAGAATGAATTTGCTGGAAATATTTCT
GCGCTGGCTGATGCGGAAAATATTTCACGTAAGATTATTACCCGCTGTATCAACA
CCGCCAAATTGCCTAAATCAGTTGTTGCTCTTTTTTCTCACCCCGGTGAACTATCT
GCCCGGTCAGGTGATGCACTTCAAAAAGCCTTTACAGATAAAGAGGAATTACTT
AAGCAGCAGGCATCTAACCTTCATGAGCAGAAAAAAGCTGGGGTGATATTTGAA
GCTGAAGAAGTTATCACTCTTTTAACTTCTGTGCTTAAAACGTCATCTGCATCAA
GAACTAGTTTAAGCTCACGACATCAGTTTGCTCCTGGAGCGACAGTATTGTATAA
GGGCGATAAAATGGTGCTTAACCTGGACAGGTCTCGTGTTCCAACTGAGTGTATA
GAGAAAATTGAGGCCATTCTTAAGGAACTTGAAAAGCCAGCACCCTGATGCGAC
CACGTTTTAGTCTACGTTTATCTGTCTTTACTTAATGTCCTTTGTTACAGGCCAGA
AAGCATAACTGGCCTGAATATTCTCTCTGGGCCCACTGTTCCACTTGTATCGTCG
GTCTGATAATCAGACTGGGACCACGGTCCCACTCGTATCGTCGGTCTGATTATTA
GTCTGGGACCACGGTCCCACTCGTATCGTCGGTCTGATTATTAGTCTGGGACCAC
GGTCCCACTCGTATCGTCGGTCTGATAATCAGACTGGGACCACGGTCCCACTCGT
ATCGTCGGTCTGATTATTAGTCTGGGACCATGGTCCCACTCGTATCGTCGGTCTG
ATTATTAGTCTGGGACCACGGTCCCACTCGTATCGTCGGTCTGATTATTAGTCTGG
AACCACGGTCCCACTCGTATCGTCGGTCTGATTATTAGTCTGGGACCACGGTCCC
ACTCGTATCGTCGGTCTGATTATTAGTCTGGGACCACGATCCCACTCGTGTTGTCG
GTCTGATTATCGGTCTGGGACCACGGTCCCACTTGTATTGTCGATCAGACTATCA
GCGTGAGACTACGATTCCATCAATGCCTGTCAAGGGCAAGTATTGACATGTCGTC
GTAACCTGTAGAACGGAGTAACCTCGGTGTGCGGTTGTATGCCTGCTGTGGATTG
CTGCTGTGTCCTGCTTATCCACAACATTTTGCGCACGGTTATGTGGACAAAATAC
CTGGTTACCCAGGCCGTGCCGGCACGTTAACCGGGCTGCATCCGATGCAAGTGTG
TCGCTGTCGACGAGCTCGCGAGCTCGGACATGAGGTTGCCCCGTATTCAGTGTCG
CTGATTTGTATTGTCTGAAGTTGTTTTTACGTTAAGTTGATGCAGATCAATTAATA
CGATACCTGCGTCATAATTGATTATTTGACGTGGTTTGATGGCCTCCACGCACGTT
GTGATATGTAGATGATAATCATTATCACTTTACGGGTCCTTTCCGGTGATCCGAC
AGGTTACGGGGCGGCGACCTCGCGGGTTTTCGCTATTTATGAAAATTTTCCGGTT
TAAGGCGTTTCCGTTCTTCTTCGTCATAACTTAATGTTTTTATTTAAAATACCCTCT
GAAAAGAAAGGAAACGACAGGTGCTGAAAGCGAGCTTTTTGGCCTCTGTCGTTT
CCTTTCTCTGTTTTTGTCCGTGGAATGAACAATGGAAGTCCGAGCTCATCGCTAAT
AACTTCGTATAGCATACATTATACGAAGTTATATTCGA.

B. Preparation of Fosmid Clones for Desired Sequencing Coverage

The ShaRc method was validated using an existing Three-spine Stickleback fish (Gasterosteus aculeatus) fosmid genome library that had been previously arrayed in frozen 384-well glycerol plates and sequenced using traditional Sanger-capillary methods. First, 1,000 glycerol plate copies from this fosmid library were pooled to create a total of 384,000 unique clones. Specific fosmid clones were then purified using conventional alkaline lysis mega prep techniques. This provided a controlled pool with a precise number of known fosmid clones corresponding to a 22× physical coverage of the 675 Mb Stickleback genome. Following mega prep, 100 μg of each purified fosmid clone was used as input to prepare a ShaRc library. Each prepared ShaRc library comprised hundreds of thousands of copies of each fosmid clone. Although it is not necessary to understand the mechanism of an invention, it is believed that this over-sampling of prepped genomic Stickleback DNA maximized the chance of recovering the majority of clones even after inherent downstream losses resulting from shearing, size selection, purification, and/or re-circularization.

A human fosmid genome library similarly processed to produce sufficient ShaRc fosmid clones comprising a 30× physical coverage of the genome, represented by more than 2.25 million unique fosmid clones. A well characterized human control DNA (CEPH/UTAH NA12878, Coriell Cell Repsitories) was chosen to facilitate method evaluation for both mammalian de novo assembly and structural rearrangement detection. Specifically, a standard human fosmid genome library was made using 20 μg of human genomic DNA. After packaging and transfection, more than 3 million fosmid colonies were plated to allow for losses in unique clones during subsequent steps. The collected human fosmid clones were purified by alkaline lysis mega prep yielding approximately 5 mg of source material. Typically, approximately 100 μg of each human fosmid clone was used to create a human ShaRc fosmid fragment library.

The ShaRc fosmid fragment method was next performed using a large-scale mammalian assembly project for the mouse (C57BL/6). fosmid genome libraries were prepared and clones prepped in the same manner as described above, resulting in over 1 million fosmid genome colonies supporting the ShaRc fosmid fragment clone preparations that yielded over 3 mg of DNA sufficient for thirty attempts using 100 μg of source material for each ShaRc fosmid fragment clone.

C. Hydroshearing

In one embodiment, the present invention contemplates a method for preparing ShaRc fosmid fragments comprising shearing purified circular fosmid DNA, wherein the circular fosmid DNA comprises genomic DNA. In one embodiment, the purified fosmid DNA is approximately 100 μg. In one embodiment, the shearing comprises hydroshearing. In one embodiment, the hydroshearing creates fosmid fragments ranging between approximately 8-10 kb. In one embodiment, the fosmid fragments comprise a pEpiFos-5 vector (i.e., for example, ˜7.5 kb) are flanked by a genomic DNA sequence (e.g., an insert). In one embodiment, each flanking genomic DNA sequence comprises several hundred base pairs.

Although it is not necessary to understand the mechanism of an invention, it is believed that the hydroshearing step may be optimized by identifying a set of parameters including, but not limited to, orifice size, speed, cycles, and input concentration that would provide the maximum fosmid fragment yield in a size range between approximately 1-100 kb, preferably between approximately 3-50 kb, more preferably between approximately 5-25 kb, but most preferably between approximately 8-10 kb. In one embodiment, when using any vector, the hydroshearing creates a first portion and a second portion. In one embodiment, the first portion comprises the vector sequence and is flanked by genomic nucleic acids. In one embodiment, the second portion is the vector base pair length less the selected vector backbone and less approximately 50 base pairs on either side of the vector backbone.

Even with these optimized hydroshear conditions an additional agarose size fractionation step to isolate fosmid fragments may be performed because improvement in fragment size uniformity increases the efficiency of the subsequent circularization step while reducing total DNA yield. For example, with 100 μg fosmid genomic clone DNA entering a hydroshear step, approximately 6-8 μg of DNA in the 8-10 kb size range was typically recovered after gel electrophoresis and extraction. Nevertheless, other hydroshear parameter screening experiments have shown the potential to increase the desired 8-10 kb range under conditions such that the additional agarose size fractionation step can be eliminated. This improvement increases total fosmid fragment yield and simplifies the overall workflow.

D. Re-Circularization & PCR Selection of Fosmid Ends

In one embodiment, the method further comprises re-circularizing the fosmid fragment, wherein the flanking genomic DNA sequences ligate together to form a paired end junction. In one embodiment, the paired end junction is a ShaRe fosmid fragment. In one embodiment, the ShaRe fosmid fragment is approximately 40 kb.

In some embodiments, re-circularizing a plurality of size-selected fosmid fragments to create a plurality of ShaRe fosmid fragments. In one embodiment, the method further comprises treating the plurality of ShaRe fosmid fragments with plasmid-safe DNAse to remove un-circularized linear DNA. In one embodiment, the plurality of fosmid fragments range between approximately 6-8 μg. After re-circularization, the ShaRe fosmid fragments are enriched using conventional PCR techniques. In one embodiment, the enrichment comprises a primer pair specific for the pEpiFos-5 vector primer binding sites adjacent to the genomic insert sequences. Although it is not necessary to understand the mechanism of an invention, it is believed that by using these primer pairs, only ShaRc fosmid fragments containing both ends of the pEpiFos-5 vector will amplify, thereby creating amplicons for the desired paired end junction size (i.e., for example, an ˜40 kb paired end junction amplicon). For example, the primer pair will not bind to circular fosmid fragments having an incompatible conformation including, but not limited to, an insert only or fragments with a single end, such that no amplicons are generated.

The primer binding sites further comprise an Illumina paired-end adapter sequence tail. Although it is not necessary to understand the mechanism of an invention, it is believed that such a primer tail removes the need for downstream 3′ adenylation and adapter ligation steps during ShaRe fosmid fragment library construction and/or sequencing. While primer hetero- and homodimer complexes were found in initial ShaRe development, optimization of the ShaRc PCR enrichment conditions significantly reduced the amount of dimer generated through reduced primer concentrations by: i) switching to a hot start polymerase; or ii) increasing the primer annealing temperature to increase primer binding stringency.

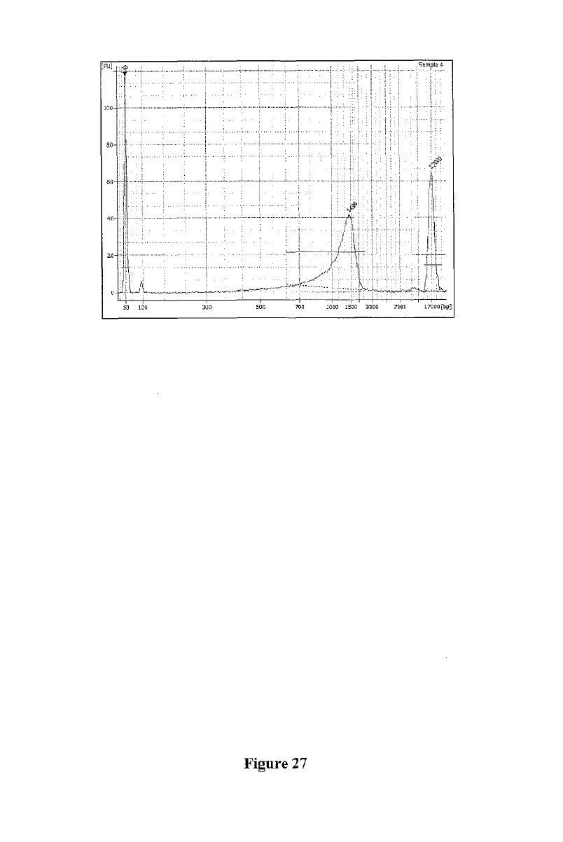

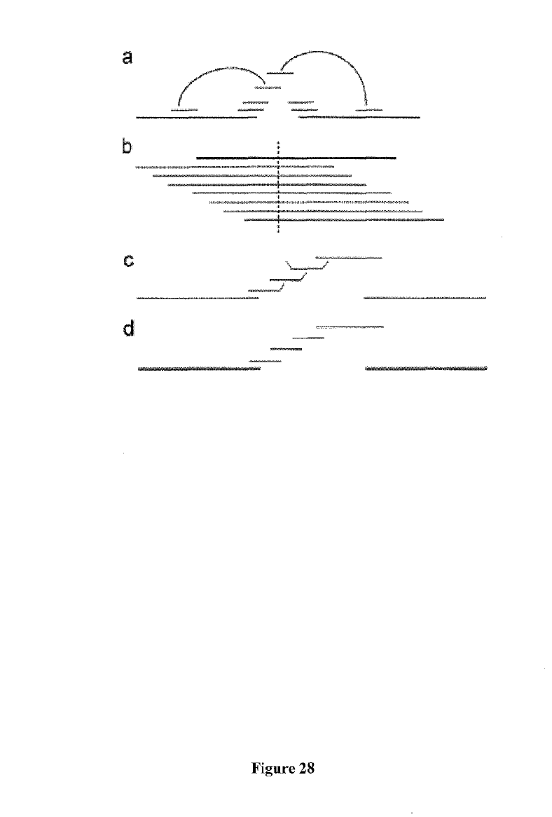

ShaRe fosmid fragments were tailed with Illumina adapter sequences ranging between approximately 500-2000 bp with an average size of 1500 bp, and when analyzed, an electropherogram presents a distinctive shark fin shape distribution. See, FIG. 27. A small adapter dimer peak can be seen at ˜100 bp. This dimer peak was reduced to this low level by optimizing the PCR reaction via the use of a hot start enzyme, reduced primer concentration, and increased primer annealing temperature. A final agarose gel size selection was performed to remove the unamplified circular DNA and primer dimer products, as well as refine sizing to 500-1000 bp to ensure optimal cluster generation and sequencing in an Illumina GAIL workflow.

E. ShaRc Library Sequencing

The data presented herein was based upon a size selected ShaRc library prepared for Illumina sequencing. A final enrichment of ShaRe fosmid fragments comprising binding sites for Illumina paired end enrichment primers 1.0 and 2.0 were added to the P5 and P7 sequences, flanking the fosmid vector sequence required for cluster amplification. Although it is not necessary to understand the mechanism of an invention, it is believed that the PCR enrichment step described herein amplifies the entire volume fosmid library clones thereby maintaining a high level of library complexity. Prior to cluster amplification, the libraries were quantified with QPCR, normalized, denatured, and loaded on a GAIIx paired end flowcell. Because each read begins with 20 bases of pEpiFos-5 sequence, all ShaRc lanes were loaded at a lower target density of less than 350,000 clusters per mm². Monotemplate stretches especially at the beginning of reads can cause problems for the base calling matrix and cluster finding algorithms in the GAIIx, and loading at a lower density allows for better cluster detection and yield for monotemplate sequencing.

F. ShaRc Analysis

Once the ShaRc fosmid fragments were sequenced, the data was first filtered for reads containing the initial 20 bases of pEpiFOS-5 sequence and then trimmed. For genomes with an available reference sequence, read pairs were aligned to calculate the jump length distribution and to filter any individual reads where a junction occurs. For some data sets described herein, if no junction was found in each read 76 bp paired-end GAII read lengths were trimmed to create an approximate 56 bp read length. In some embodiments, a longer paired-end read length of approximately 101 bp is preferable to provide longer alignment lengths.

For the 384,000 Three-spine Stickleback fosmid clone preparation described above, two ShaRc fosmid fragment libraries were created, each starting with 100 μg of the mega prepped clones. Size-selected ShaRc fosmid paired end fragments of approximately 76 bp from each library were sequenced in a single lane. Each lane was loaded with the optimized lower density (i.e., for example, <300,000 clusters per mm²) to accommodate the monotemplate sequence originating from the pEpiFos-5 vector at the beginning of each read. These reads were then trimmed and duplicate reads eliminated. The trimmed pairs were then aligned to the draft Stickleback reference sequence.

Production of the Stickleback ShaRc fosmid fragments from the first and second libraries produced a clone recovery of 82.9% (314,428 unique) and 85.5% (328,464 unique), respectively. The chimerism rate was <13% in each library data set. Although it is not necessary to understand the mechanism of an invention, it is believed that the chimerism rate is likely a result of alignment artifacts and a draft reference for alignment. However, after combining the two libraries a nearly 100% recovery of the original 384,000 clones is obtained. While nearly all the ShaRc fosmid paired end sequences were recovered, a large percentage (˜39%) of reads aligned at an insert size of less than 1 kb. Although it is not necessary to understand the mechanism of an invention, it is believed that these aberrant read pairs were likely due to orphan reads where only a single end could be placed or caused by an inefficient exonuclease removal of linear fragments after re-circularization. In some embodiments described herein, the plasmid-safe exonuclease step has been optimized to minimize this problem.

To verify that the Stickleback ShARC data was comparable to traditional Sanger-capillary fosmid end-sequencing, an existing assembly of the Trichophyton genome (˜22 Mb; ABI 3730 was compared (i.e., plus versus minus) to ShaRc fosmid data using Velvet®. See, Table 3.

TABLE 3
Comparative Stickleback Data: ShaRc
Versus Sanger-Capillary Sequencing

| Trichophyton | Fosmid | | | |
|---|---|---|---|---|
| Assembly | Sequence | | Scaffold | Reference |
| (Velvet) | Coverage | Scaffolds | N50 | Covered |
| ABI 3730 Only | 0 | 466 | 62,900 | 86.42% |
| w/o Fosmids | | | | |
| ABI 3730 w/ | 2.75 | 28 | 2,134,475 | 97.52% |
| ABI Fosmids | | | | |
| ABI 3730 w/ | 1.25 | 54 | 2,125,345 | 83.74% |
| ShARC Fosmids | | | | |

The Illumina GAII ShARC data produced comparable results to the ABI only assembly at less than half the sequence coverage but also over 1000× lower cost.

Mouse ShaRc fosmid fragment GAIT sequencing was similarly compared against Sanger-capillary sequencing. This assessment evaluated the performance of the methods described herein on a large mammalian genome (>1 Gb). A mouse fosmid clone genomic library was used to create four ShaRc fosmid mouse genome fragment libraries as described above, using approximately 100 μg of fosmid library source material for each ShaRc library. The ShaRc fosmid mouse genome fragment libraries were sequenced on GAIIs using a 76 bp paired-end low-density protocol as described above for the Stickleback analysis. The resulting Illumina data reads were trimmed to 48 bp to ensure that only genomic insert remained. A genome reconstruction assembly (ALLPATHS; infra) demonstrated a 2.1× in sequence coverage with the presence of ShaRc fosmids as compared to 180 bp paired end only Illumina data. The presence of ShaRc fosmids also more than doubled the Scaffold N50 size, and was performed at a much lower cost than an equivalent Sanger-capillary data set. See Table 4.

TABLE 4
Comparative Illumina Sequencing For The Mouse Genome

| | fosmid | |
|---|---|---|
| Mouse Assembly | Sequence | Scaffold |
| (Allpaths) | Coverage | N50 |
| Illumina PE Only | 0 | 2.57 Mb |
| w/o fosmids | | |
| Illumina PE w/ | 2.1 | 5.47 Mb |
| ShARC fosmids | | |

Human ShaRe fosmid fragments were also produced yielding similar yields and results. A human fosmid clone genomic library was used to create two ShaRc fosmid fragment human genomic libraries using approximately 100 μg of fosmid genome library clone as source material for each library.

The human ShaRc fosmid genomic libraries were sequenced on GAIIs using a 76 bp paired-end low-density protocol, and the resulting data was trimmed of the vector sequence. Two lanes of a flowcell from both libraries were sequenced (yielding over 1.9 M unique clones) and the data was combined. See, Table 5.

TABLE 5
Combined Sequencing Data From Two Human ShaRc fosmid Fragment Libraries

| | | PF Aligned | Unique Insert | Unique Insert | | Physical | % |
|---|---|---|---|---|---|---|---|
| Library | Lane | Pairs | 0-1 kb | 30-50 kb | Median Size | Coverage (x) | Chimeras |
| 1 | 1 | 7,000,455 | 309,628 | 1,201,931 | | 15.5 | 4.04% |
| 1 | 2 | 7,060,914 | 319,007 | 1,137,811 | | 14.7 | 3.95% |
| 2 | 1 | 3,830,597 | 273,696 | 738,612 | | 9.5 | 4.04% |
| 2 | 2 | 3,339,412 | 266,543 | 656,246 | | 8.5 | 4.05% |
| Library | Lane | 10,831,052 | 583,324 | 1,940,543 | | 25.0 | 4.04% |
| 1 + 2 | 1 + 2 | | | | | | |

Both human ShaRc fosmid fragment libraries started from a common fosmid library prep so the clones in each library, and each lane, overlap when combined in a single set. An estimated 3 million human ShaRc fosmid fragment clones were plated and scraped based on colony counts from a sample of plates, which translated into an approximate 65% recovery rate. An alignment analysis suggests that the chimerism rate is comparable to that found in Sanger-capillary data sets. Although it is not necessary to understand the mechanism of an invention, it is believed that chimerism rates may be affected in ShaRc data sets by the alignment of individual reads (i.e., forward or reverse) that contain a junction point. In one embodiment, the present invention contemplates a nucleic acid sequence (i.e., for example, a stuffer nucleic acid sequence) thereby clearly demarcating the junction point added at re-circularization step. Although it is not necessary to understand the mechanism of an invention, it is believed that the integration of a staffer sequence minimizes ShaRc chimerism rates.

### IV. Bar Coding

A. ShARC Barcoding

In one embodiment, the present invention contemplates a set of vector sequences (i.e., for example, a set of pFosCN-2 vector sequences) wherein each vector sequence comprises a unique synthetic nucleic acid sequence that serves as a barcode identifier. In one embodiment, the set of barcodes has been computationally designed to have the maximum information entropy and set properties such that each barcode is as distinct from the others as possible. Further, the set is tolerant to individual barcodes failing while still retaining maximum diversity. In one embodiment, the barcode sequences are selected to not have matches to known genomic sequences including but not limited to human, mouse, and microbial (i.e., for example, bacterial). In one embodiment, multiple pFosCN-1 or pFosCN-2 vectors comprise unique barcodes that identify the sample cloned into each vector. In one embodiment, each barcode is uniquely associated to a genome or genomic DNA sample.

To address the potential needs of bacterial assembly and to minimize the number of fosmid libraries created for any given set of organisms, some embodiments of the present invention contemplate a barcoding method for multiplexing a number of samples into a single fosmid prep. Such a multiplexed fosmid genome clone preparation could then be processed by the methods described herein to create a multiplexed ShaRc fosmid fragment genomic library. For example, data is presented based upon an experimental design to multiplex 6 microbial species in the same fosmid clone library. See, Table 6.

TABLE 6
Multiplexing Of Microbial Genomes By Barcoding

| | | | Percent |
|---|---|---|---|
| | Barcode | Read | of |
| Organism | Sequence | Pairs | Total |
| Bifidobacterium | TTGAGCCT | 1,488,522 | 15.5% |
| bifidum | | | |
| Streptomyces | AGTTGCTT | 217,188 | 2.26% |
| roseosporus | | | |
| Neisseria | CCAGTTAG | 3,778,961 | 39.34% |
| gonorrhoeae MS11 | | | |
| Neisseria | ACCAACTG | 412,0121 | 42.89% |
| gonorrhoeae FA19 | | | |
| Enterococcus | GTATAACA | 32 | 0.00% |
| faecium | | | |
| Parabacteroides sp. | CAGGAGCC | 518 | 0.01% |

A sample of a fosmid genomic clone library from each species was hydrosheared to a target 40 kb, agarose gel size-selected for 35-45 kb fragments and end repaired, wherein the corresponding barcode adapters were added. Aliquots from each species barcoded fosmid genomic fragment library sample were then pooled to total 20 μg and used as input to a single fosmid library. The pooled fosmid library was used to create a single ShARC fosmid fragment genomic library and sequenced on GAIIs using a 76 bp paired-end low-density protocol, wherein the resulting data was trimmed of the vector sequence. A single flowcell lane from the library yielded over 5.6 million aligned pairs with an identifiable matching barcode on both reads, although the majority came from just 4 of the barcoded samples. Although it is not necessary to understand the mechanism of an invention, it is believed that this bias was likely due to an inefficient adapter ligation in 2 of the samples and volume based pooling rather than pooling on an equimolar basis. Further, a high number of duplicates was also found in the read pairs which is likely a function of the large number of reads relative to a small genome size. The data demonstrate that multiple organisms can be pooled using barcodes and properly sequenced using the ShaRe method.

B. fosIll Barcoding

In one embodiment, the present invention contemplates a method comprising providing PCR primers to introduce at least one 8-base barcode into a pfosIII-3-derived fosIll-library. In one embodiment, several (for example 8) fosmid libraries are constructed using pfosill-3. Each library is then converted to a barcoded fosill library. In one embodiment, the barcode is introduced during an inverse PCR step. In one embodiment, the primer comprises a forward primer. In one embodiment, the forward primer comprises an Illumina PE1.0 enrichment primer. In one embodiment, the forward primer is AATGATACGGCGACCACCGAGATCTA-CACTCTTTCCCTACACGACGCTCTTCCGATCT. In one embodiment, the primer comprises a reverse primer. In one embodiment, the reverse primer comprises a reverse sequencing primer. In one embodiment, the reverse sequencing primer comprises a barcode. In one embodiment, the reverse sequencing barcoded primer may include but is not limited to: i) FIR_{—}375 CAAGCAGAAGACGGCATACGAGATTAAGCAT-GGTGACTGGAGTTCAGACGTGTGC; ii) FIR_{—}190 CAAGCAGAAGACGGCA-TACGAGATGAATTGCTGTGACTGGAGTTCAGACGTGTGC; iii) FIR 504 CAAGCAGAAGACGGCATACGAGATCCTGGTAGGTGACTGGAGTTCAGACGTGTG C; iv) FIR_{—}236 CAAGCAGAAGACGGCATACGAGATAAGCAACTGTG-ACTGGAGTTCAGACGTGTGC; v) FIR_{—}908 CAAGCAGAAGACGGCATA-CGAGATGTCGAGCAGTGACTGGAGTTCAGACGTGTGC; vi) FIR 630 CAAGCA-GAAGACGGCATACGAGATAGATGTGCGTGACTGGAGTTCAGACGTGTGC; vii) FIR 960 CAAGCAGAAGACGGCATACGAGATAGGCTCAAGTGACTGGAGTT-CAGACGTGTGC; and viii) FIR_{—}393 CAAGCAGAAGACGGCATACGAGATCTAA-CTGGGTGACTGGAGTTCAGACGTGTGC (underlining indicates the primer barcode sequence).

For example, data is presented using this embodiment to convert 8 fosmid libraries in pfosill-3 to 8 barcoded fosill libraries. See, Table 7.

TABLE 7
Multiplex sequencing of 8 barcoded fosill libraries

| Barcode on | Correct |
|---|---|
| fosIll | (30-to-50 Kb) |
| library | jumps |
| 190 | 3,668,689 |
| 236 | 3,613,523 |
| 375 | 4,618,609 |
| 393 | 2,074,416 |
| 504 | 3,142,218 |
| 603 | 2,528,530 |
| 908 | 2,618,316 |
| 960 | 3,080,541 |

In one embodiment, the present invention contemplates a method comprising providing a fosmid cloning vector pfosill-4 and generating a barcoded fosIll library. In one embodiment, the method further comprises ligating barcoding adapters to genomic DNA fragments. In one embodiment, the method further comprises pooling different DNA fragments (i.e., for example, fragments derived from different genomes), wherein each different genomic DNA fragment is ligated to a different unique barcode. In one embodiment, the method further comprises cloning the different genomic DNA fragments to create a single, multiplex barcoded fosmid library which is then converted to a single multiple barcoded fosill library.

The barcoded fosIll library may be made by various methods, including but not limited to:

Step 1: Digesting pfosIll-4 using AatII and SapI. Although it is not necessary to understand the mechanism of an invention it is believed that this digestion results in two vector “arms” with shortened Illumina adapters (shaded area) as shown below only from the BbvCI nicking site to the Illumina adapter sequences:

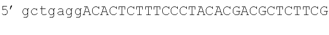

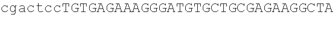

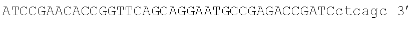

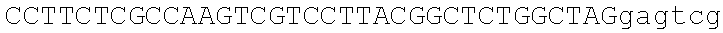

As shown, it is believed that SapI cuts twice within the Illumina adapter region (shaded area) and leaves a 3 bp 5′ overhang.

Step 2. Ligating the insert into a sheared and end-repaired genomic DNA using a phosphorylated 8 bp barcoded oligonucleotide. As illustrated below an 8 bp barcode sequence (e.g., a 375 barcode sequence) are attached to an Illumina adapter on both terminal ends of the insert creating a trinucleotide overhang:

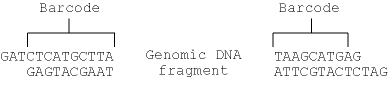

Step 3. Pooling the barcoded inserts.

Step 4: Size selecting the pooled barcoded inserts using, for example, a pulse field gel. Although it is not necessary to understand the mechanism of an invention, it is believed that such size selection creates fewer short “non-jumps”, as shown in several genomic nucleic acid studies. See, FIGS. 29A, 29B, and 29C. These data show that when using the 8 bp barcoded DNA inserts, such size selection methods have demonstrated a high frequence of correct 30-50 kb jumps.

Step 5: Ligating the size selected barcoded inserts to a fosIll-4 vector arm to create a fosIll ligation product capable of creating a fosIll library that can be sequenced with paired-end Illumina primers without a constant, vector derived, “mono-template” at the beginning of each read:

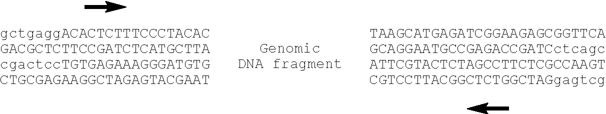

Step 6. Constructing a barcoded fosIll library using a fosIll ligation product as depicted above. See, FIG. 29.

In one embodiment, an alternative method provides barcoding adapter sequences that may be ligated after size-selecting the sheared and end-repaired genomic DNA. In one embodiment, the barcoding adapter sequences may comprise (the 8 bp barcode sequence is indicated by underline):

375-SapI:
GATCTCATGCTTA
GAGTACGAAT,
190-SapI:
GATCTAGCAATTC
GATCGTTAAG,
504-SapI:
GATCTCTACCAGG
GAGATGGTCC,
236-SapI:
GATCTAGTTGCTT
GATCAACGAA,
908-SapI:
GATCTTGCTCGAC
GAACGAGCTG,
630-SapI:
GATCTGCACATCT
GACGTGTAGA,
t960-SapI:
GATCTTTGAGCCT
GAAACTCGGA,
or
t393-SapI:
GATCTCCAGTTAG
GAGGTCAATC.

Such variations of barcoded adapter sequences can be used to ligate an insert DNA sequence providing the steps including but not limited to:

adding a 3′ T to a first strand of a barcoded adapter sequence; and

dephosphorylating a second strand of the barcoded adapter sequence

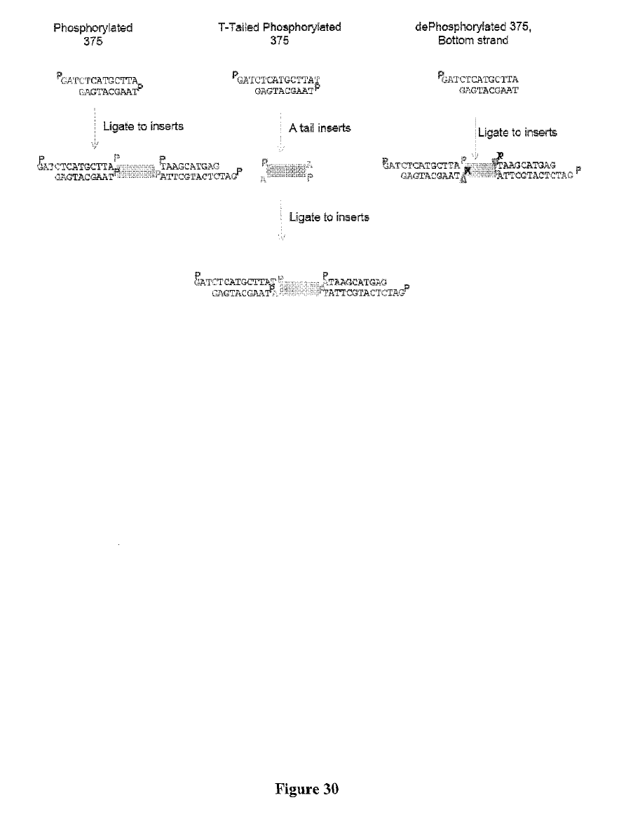

Although it is not necessary to understand the mechanism of an invention, it is believed that these steps may: i) reduce chimeras due to ligation of inserts to inserts, as the inserts will have a 3′ dATP added; ii) reduce adapter-dimer formation; and/or iii) reduce adapter-dimer formation. The various methods discussed above, using barcode 375 as an example, are compared herein. See, FIG. 30.

### V. Nucleic Acid Sequencing

The first DNA sequences were obtained using laborious methods based on two-dimensional chromatography. Following the development of dye-based sequencing methods with automated analysis, DNA sequencing has become easier and faster. Olsvik et al., “Use of automated sequencing of polymerase chain reaction-generated amplicons to identify three types of cholera toxin subunit B in Vibrio cholerae 01 strains” J. Clin. Microbiol. 31:22-25 (1993).

A. Chain Termination Sequencing

The chain-termination method (i.e., for example, the Sanger method) introduced improvements into nucleotide sequencing technology by increasing efficiency, reducing the use of toxic chemicals and/or radioactivity than initial techniques. Chain termination sequencing introduced the use of dideoxynucleotide triphosphates (ddNTPs) as DNA chain terminators.

A classical chain-termination method usually comprises a single-stranded DNA template, a DNA primer, a DNA polymerase, radioactively or fluorescently labeled nucleotides, and modified nucleotides that terminate DNA strand elongation. The DNA sample is divided into four separate sequencing reactions, containing all four of the standard deoxynucleotides (e.g., dATP, dGTP, dCTP and dTTP) and the DNA polymerase. To each reaction is added only one of the four dideoxynucleotides (e.g., ddATP, ddGTP, ddCTP, or ddTTP) which are the chain-terminating nucleotides. These dideoxynucleotides lack a 3′—OH group required for the formation of a phosphodiester bond between two nucleotides, thus terminating DNA strand extension and resulting in DNA fragments of varying length.

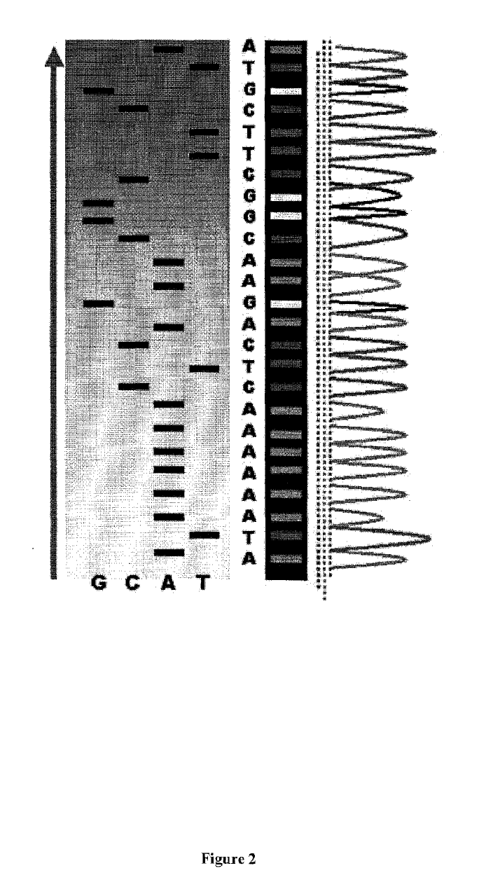

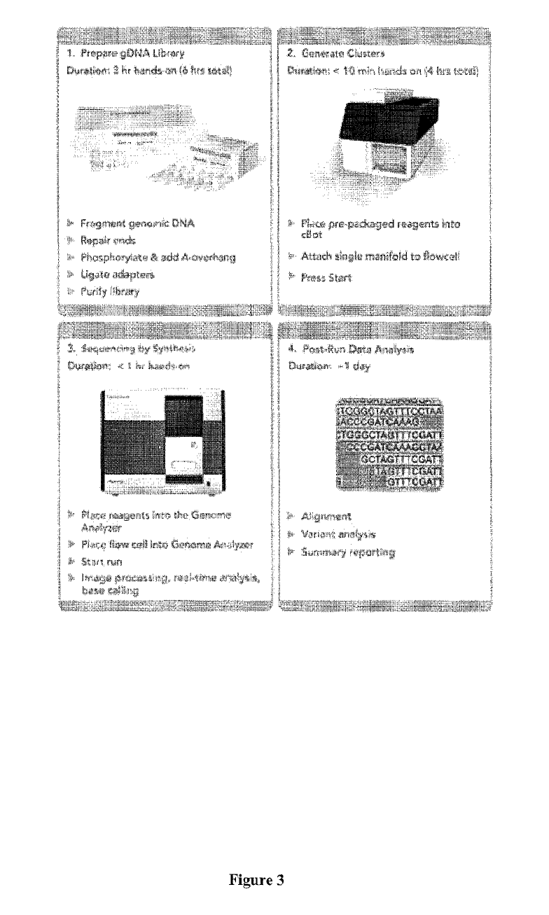

Newly synthesized and labeled DNA fragments are heat denatured, and separated by size (i.e., for example, with a resolution of just one nucleotide) by gel electrophoresis on a denaturing polyacrylamide-urea gel with each of the four reactions run in one of four individual lanes (lanes A, T, G, C); the DNA bands are then visualized by autoradiography or UV light, and the DNA sequence can be directly read off the X-ray film or gel image, wherein dark bands on the gel correspond to DNA fragments of different lengths. For example, a dark band in a lane indicates a DNA fragment that is the result of chain termination after incorporation of a dideoxynucleotide (ddATP, ddGTP, ddCTP, or ddTTP). The relative positions of the different bands among the four lanes are then used to read (from bottom to top) the DNA sequence. See, FIG. 2.

B. Next-Generation Sequencing

Next-generation sequencing technologies (i.e., for example, high throughput sequencing) parallelize the sequencing process and results in a low-cost method that simultaneously produces thousands or millions of sequences. Hall N., “Advanced sequencing technologies and their wider impact in microbiology” J. Exp. Biol. 210 (Pt 9): 1518-1525 (2007); and Church G., “Genomes for all” Sci. Am. 294: 46-54 (2006). Advantages of next-generation sequence reads including but not limited to: i) the length of a sequence read from most current next-generation platforms is shorter than that from a capillary sequencer; and ii) each next-generation read type has a unique error model different from that already established for capillary sequence reads. Both differences affect how the reads are utilized in bioinformatic analyses, depending upon the application. For example, in strain-to-reference comparisons (i.e., for example, re-sequencing), the typical definition of repeat content must be revised in the context of the shorter read length. In addition, a much higher read coverage or sampling depth is required for comprehensive resequencing with short reads to adequately cover the reference sequence at the depth and low gap size needed. Some applications are more suitable for certain platforms than others, as detailed below. Furthermore, read length and error profile issues entail platform- and application-specific bioinformatics-based considerations. Moreover, it is important to recognize the significant impacts that implementation of these platforms in a production sequencing environment has on informatics and bioinformatics infrastructures.

Several techniques for massively parallel DNA sequencing have recently been described. Ronaghi et al., “Analyses of secondary structures in DNA by pyrosequencing” Anal Biochem 267: 65-71 (1999); Brenner et al., “Gene expression analysis by massively parallel signature sequencing (MPSS) on microbead arrays” Nat Biotechnol 18:630-634 (2000); Braslaysky et al., Sequence information can be obtained from single DNA molecules” Proc Natl Acad Sci 100:3960-3964 (2003); Margulies et al., “Genome sequencing in microfabricated high-density picolitre reactors” Nature 437:376-380 (2005); Shendure et al., “Accurate multiplex polony sequencing of an evolved bacterial genome” Science 309:1728-1732 (2005); Ju et al., “Four-color DNA sequencing by synthesis using cleavable fluorescent nucleotide reversible terminators” Proc Natl Acad Sci 103:19635-19640 (2006); Gibbs et al., “Evolutionary and biomedical insights from the rhesus macaque genome” Science 316:222-234 (2007); Bentley et al., “Accurate whole human genome sequencing using reversible terminator chemistry” Nature 456:53-59 (2008); and Eid et al., “Real-time DNA sequencing from single polymerase molecules” Science 323:133-138 (2009).

These techniques broadly fall into at least two assay categories (i.e., for example, polymerase and/or ligase based) and/or at least two detection categories (i.e., for example, asynchronous single molecule and/or synchronous multi-molecule readouts). For example, SOLiD (Sequencing by Oligo Ligation Detection) sequencing comprises a DNA ligase-based synchronous ensemble detection method utilized to read 500 million to over 1 billion reads per instrument run. Cloonan et al., “Stem cell transcriptome profiling via massive-scale mRNA sequencing” Nat Methods 5:613-619 (2008); and Valouev et al., “A high-resolution, nucleosome position map of C. elegans reveals a lack of universal sequence-dictated positioning” Genome Res 18:1051-1063 (2008).

All of these techniques are theoretically compatible with mate-paired sequencing, but they differ in how they generate the mate-paired reads. For example, one approach generates short pairs from cluster polymerase chain reaction (PCR) colonies often referred to as “paired-ends.” Campbell et al., “Identification of somatically acquired rearrangements in cancer using genomewide massively parallel paired-end sequencing” Nat Genet 40: 722-729 (2008). These paired-end reads have limited insert sizes due to the efficiency and representation of PCR amplification of long amplicons via cluster PCR. Consequently, very few paired-end reads are generated that are longer than a Sanger capillary electrophoresis read (<10³ clone coverage in pairs >1.0 kb).

DNA circularization and random shearing have been used, thereby circumventing the need to PCR amplify the entire pairing distance at the cost of more input DNA. Korbel et al., “Paired-end mapping reveals extensive structural variation in the human genome” Science 318: 420-426 (2007); and Bentley et al., “Accurate whole human genome sequencing using reversible terminator chemistry” Nature 456:53-59 (2008). These pairs each differ substantially in their tag length due to the random shearing step. The asymmetrical tag lengths reduce the pairing efficiency and often contaminate the library prep with a high number of 200-bp inserts; thus, no more than 100× clone coverage is obtained, and many tags are sequenced that are not paired or are paired in the wrong distance or orientation. Furthermore, these techniques may result in many inverted molecules that complicate the detection of inversions.

A preferred pairing method would provide both high sequence coverage and high clone or “physical” coverage with flexible insert sizes such that SNPs, small indels, larger structural variations, and copy number variants (CNVs) could be surveyed in one method. Two pairing methods can be used that retain less variable tag lengths while enabling both high sequence coverage and high clone coverage of the human genome to enable the broadest survey of variation possible. Use of ligases for massively parallel short-read DNA sequencing of human genomes offers several unique attributes next to polymerases. Most notable is the use of an error-correcting probe-labeling scheme (two-base encoding, or 2BE), which provides error correction concurrent with the color-called alignment of the data (i.e., for example, without having to resequence the reads). This correction property has specific utility in bisulfate sequencing, de novo assembly, indel detection, and SNP detection.

SOLiD sequencing is believed capable of efficiently surveying single nucleotide polymorphisms and many forms of structural variation concurrently at relatively modest coverage levels. Such an expansive clone coverage allows identification of a larger number of structural variants in a size range not efficiently explored in previous studies.

The massively parallel scale of sequencing implies a similarly massive scale of computational analyses that include image analysis, signal processing, background subtraction, base calling, and quality assessment to produce the final sequence reads for each run. See, Example III. In every case, these analyses place significant demands on the information technology (IT), computational, data storage, and laboratory information management system (LIMS) infrastructures extant in a sequencing center, thereby adding to the overhead required for high-throughput data production. This aspect of next-generation sequencing is at present complicated by the dearth of current sequence analysis tools suited to shorter sequence read data; existing data analysis pipelines and algorithms must be modified to accommodate these shorter reads. In many cases, and certainly for new applications of next-generation sequencing, entirely new algorithms and data visualization interfaces are being devised and tested to meet this new demand. Therefore, the next-generation platforms are effecting a complete paradigm shift, not only in the organization of large-scale data production, but also in the downstream bioinformatics, IT, and LIMS support required for high data utility and correct interpretation.

This paradigm shift promises to radically alter the path of biological inquiry, as the following review of recent endeavors to implement next-generation sequencing platforms and accompanying bioinfonnatics-based analyses serves to substantiate.

Most massively parallel high throughput sequencing techniques avoid molecular cloning in a microbial host (i.e., for example, transformed bacteria, such as E. coli) to propagate the DNA inserts. Instead, they use in vitro clonal PCR amplification strategies to meet the molecular detection sensitivities of the current molecule sequencing technologies. Some sequencing platforms (e.g., Helicos Biosciences) avoid amplification altogether and sequence single, unamplified DNA molecules. With or without clonal amplification, the available yield of unique sequencing templates has a significant impact on the total efficiency of the sequencing process. Various clonal amplification methods have been described in more detail below

1. Emulsion Amplification

Emulsion PCR is generally used to isolate individual DNA molecules along with primer-coated beads in aqueous droplets within an oil phase. An ensuing polymerase chain reaction process then coats each bead with clonal copies of the DNA molecule followed by immobilization for later sequencing. Emulsion PCR is more commonly referred to as: i) 454 sequencing (Margulies et al., “Genome sequencing in microfabricated high-density picolitre reactors” Nature 437:376-380 (2005); ii) polony sequencing (Shendure, J. “Accurate Multiplex Polony Sequencing of an Evolved Bacterial Genome” Science 309:1728 (2005); and iii) SOLiD sequencing (Applied Biosystems).

454 sequencing techniques employ pyrosequencing that uses DNA polymerization by adding one nucleotide species at a time and detecting and quantifying the number of nucleotides added to a given location through the light emitted by the release of attached pyrophosphates. Ronaghi et al., “Real-time DNA sequencing using detection of pyrophosphate release” Analytical Biochemistry 242: 84-89 (1996). The SOLiD platform uses an adapter-ligated fragment library similar to those of the other next-generation platforms, and uses an emulsion PCR approach with small magnetic beads to amplify the fragments for sequencing. Unlike the other platforms, SOLiD uses

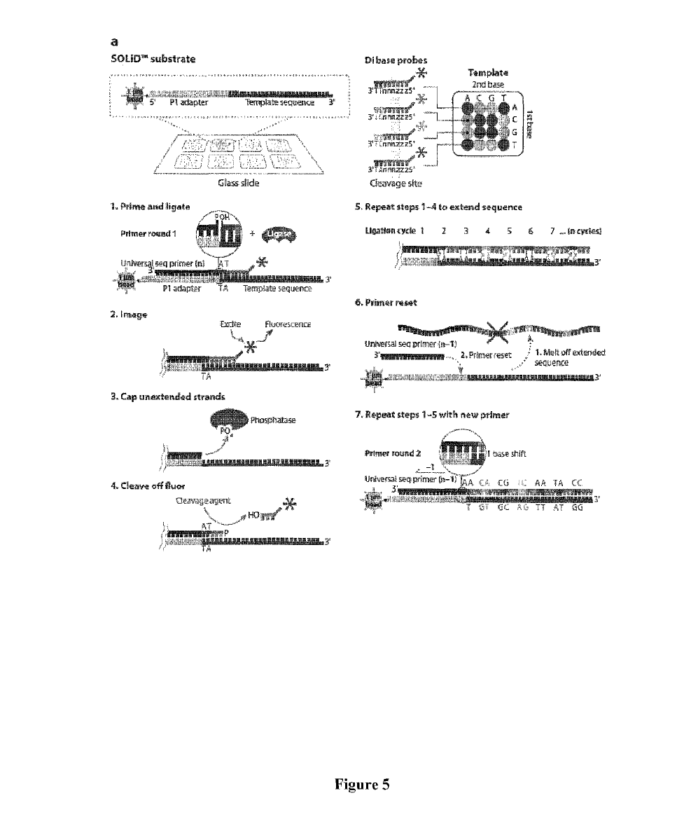

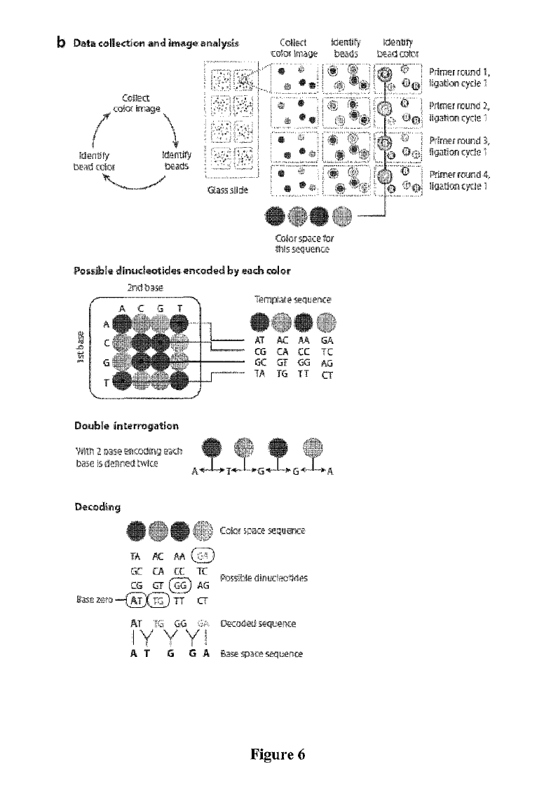

DNA ligase and a unique approach to sequence the amplified fragments. See, FIG. 5. Two flow cells are processed per instrument run, each of which can be divided to contain different libraries in up to four quadrants. Read lengths for SOLiD are user defined between 25-50 bp, and each sequencing run yields up to ˜100 Gb of DNA sequence data. Once the reads are base called, have quality values, and low-quality sequences have been removed, the reads are aligned to a reference genome to enable a second tier of quality evaluation called two-base encoding. The principle of two-base encoding illustrates how this approach works to differentiate true single base variants from base-calling errors. See, FIG. 6.

2. Bridge Amplification

Bridge PCR also involves in vitro clonal amplification, wherein the cloned fragments are amplified using primers that are attached to a solid surface. Such configurations are compatible with an Illumina Genome Analyzer. For example, DNA molecules are physically bound to a surface such that they may be sequenced in parallel (i.e., for example, known in the art as massively parallel sequencing).

Sequencing by synthesis techniques (i.e., for example, dye-termination electrophoretic sequencing) uses a DNA polymerase to determine the base sequence. Alternatively, a reversible terminator method may be used wherein fluorescently labeled nucleotides are individually added, such that each position is determined in real time (i.e., for example, Illumina). A blocking group on each labeled nucleotide is then removed to allow polymerization of another nucleotide.

Massively parallel sequencing of millions of fragments has been successfully commercialized by a reversible terminator-based sequencing chemistry (Illumina) This sequencing technology offers a highly robust, accurate, and scalable system that is cost-effective, and sufficiently accurate to support next-generation sequencing technologies. For example, the Illumina sequencing technology relies on the attachment of randomly fragmented genomic DNA to a planar, optically transparent surface. These attached DNA fragments are extended and bridge amplified to create an ultra-high density sequencing flow cell with hundreds of millions of clusters, each containing 1,000 copies of the same template. These templates are sequenced using a robust four-color DNA sequencing-by-synthesis technology that employs reversible terminators with removable fluorescent dyes. This approach ensures high accuracy and true base-by-base sequencing, eliminating sequence-context specific errors and enabling sequencing through homopolymers and repetitive sequences.

High-sensitivity fluorescence detection may be achieved using laser excitation and total internal reflection optics. Sequence reads are aligned against a reference genome and genetic differences are called using specially developed data analysis pipeline software. Alternative sample preparation methods allow the same system to be used for a range of applications including gene expression, small RNA discovery, and protein-nucleic acid interactions.

After completion of the first read, the templates can be regenerated in situ to enable a second 75+ bp read from the opposite end of the fragments. A paired-end module directs the regeneration and amplification operations to prepare the templates for the second round of sequencing. First, the newly sequenced strands are stripped off and the complementary strands are bridge amplified to form clusters. Once the original templates are cleaved and removed, the reverse strands undergo sequencing-by-synthesis. The second round of sequencing occurs at the opposite end of the templates, generating 75+ bp reads for a total of >20 Gb of paired-end data per run.

A single molecule amplification step compatible with the Illumina Genome Analyzer may start with an Illumina-specific adapter library and takes place on an oligo-derivatized surface of a flow cell. A flow cell comprises an 8-channel sealed glass microfabricated device that allows bridge amplification of fragments on its surface, and uses DNA polymerase to produce multiple DNA copies (i.e., for example, DNA clusters) wherein each cluster represents a single molecule that initiated the cluster amplification. A separate library can be added to each of the eight channels, or the same library can be used in all eight, or combinations thereof. Each cluster may contain approximately one million amplicons (e.g., copies) of the original fragment, which is sufficient for reporting incorporated bases at the required signal intensity for detection during sequencing.

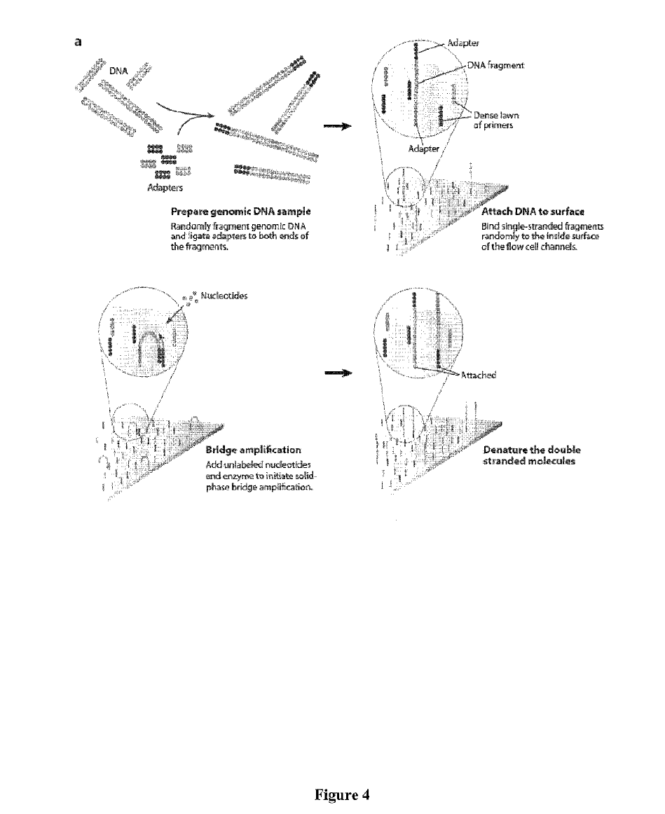

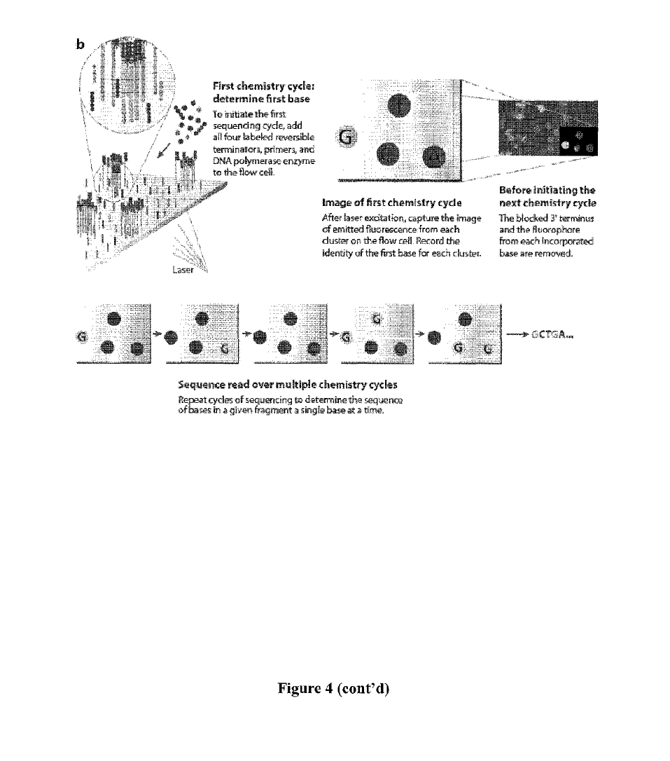

The Illumina system utilizes a sequencing-by-synthesis approach in which all four nucleotides are added simultaneously to the flow cell channels, along with DNA polymerase, for incorporation into the oligo-primed cluster fragments. See, FIG. 4. Specifically, the nucleotides carry a base-unique fluorescent label and the 3′—OH group is chemically blocked such that each incorporation is a unique event. An imaging step follows each base incorporation step, during which each flow cell lane is imaged in three 100-tile segments by the instrument optics at a cluster density per tile of 300,000 or more. After each imaging step, the 3′ blocking group is chemically removed to prepare each strand for the next incorporation by DNA polymerase. This series of steps continues for a specific number of cycles, as determined by user-defined instrument settings, which permits discrete read lengths of 75+ bases. A base-calling algorithm assigns sequences and associated quality values to each read and a quality checking pipeline evaluates the Illumina data from each run, removing poor-quality sequences.

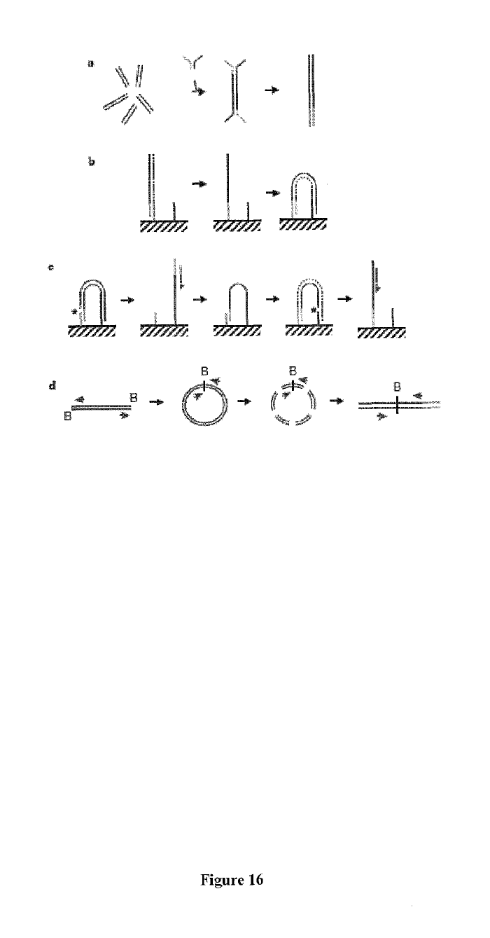

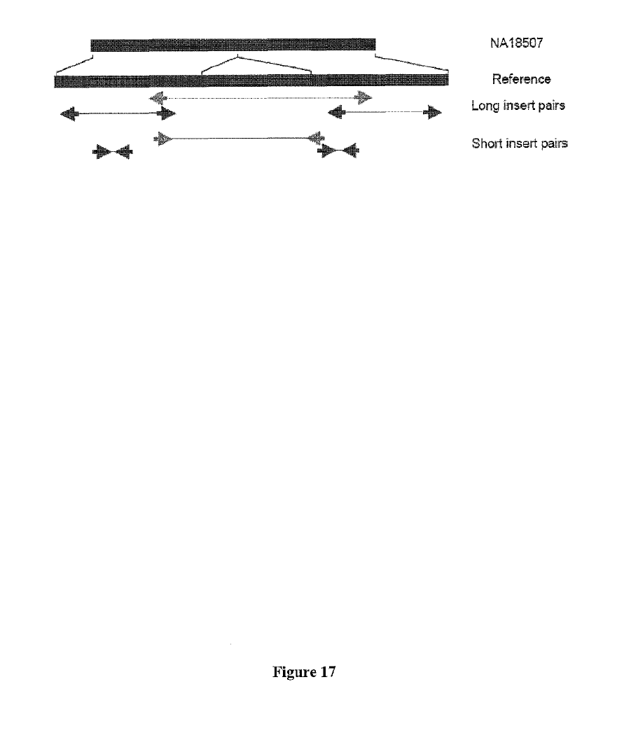

For example, a high-density single-molecule arrays of genomic DNA fragments may be attached to the surface of the flow cell reaction chamber and used isothermal ‘bridging’ amplification to form DNA ‘clusters’ from each fragment. In such an array, the DNA in each cluster single stranded and added a universal primer for sequencing. For paired read sequencing, the DNA templates are converted to double-stranded DNA and removed the original strands, leaving the complementary strand as template for the second sequencing reaction. See, FIG. 16A-16C. To obtain paired reads separated by larger distances, DNA fragments may be circularized of the required length short junction fragments are constructed to support paired end sequencing. See, FIG. 16D. Bentley et al., “Accurate whole human genome sequencing using reversible terminator chemistry” Nature 456:53-59 (2008).

C. Shotgun Sequencing

In genetics, shotgun sequencing, also known as shotgun cloning, is generally referred to as a method used for sequencing long DNA strands. It is named by analogy with the rapidly-expanding, quasi-random firing pattern of a shotgun. Since the chain termination method of DNA sequencing can only be used for fairly short strands (i.e., for example, 100 to 1000 basepairs), longer sequences must be subdivided into smaller fragments, and subsequently re-assembled to give the overall sequence. Two principal methods are used for this: chromosome walking, which progresses through the entire strand, piece by piece, and shotgun sequencing, which is a faster but more complex process, and uses random fragments.

In shotgun sequencing, DNA is broken up randomly into numerous small segments, which have been conventionally sequenced using the chain termination method to obtain reads. Multiple overlapping reads for the target DNA are obtained by performing several rounds of this fragmentation and sequencing. Computer programs then use the overlapping ends of different reads to assemble them into a continuous sequence. Staden R., “A strategy of DNA sequencing employing computer programs” Nucleic Acids Research 6: 2601-2610 (1979) and Anderson S., “Shotgun DNA sequencing using cloned DNase I-generated fragments” Nucleic Acids Research 9:3015-3027 (1981). For example, a single nucleic acid sequence may be sequenced as two separate fragments, wherein each fragment comprises two reads, the respective 3′-5′ strand and the 5′-3′ strand. None of the four different reads cover the full length of the original sequence. However, the four reads can be assembled into the original sequence using nucleic acid sequence overlap of their ends, that both to align and order the respective reads. The original shotgun sequencing method had disadvantages by necessitating the processing an enormous amount of information that generated ambiguities and sequencing errors. Assembly of complex genomes is additionally complicated by the great abundance of repetitive sequence, meaning similar short reads could come from completely different parts of the sequence.

Consequently, numerous overlapping read segments for each fragment of original DNA are necessary to overcome these difficulties and accurately assemble the sequence. For example, to complete the Human Genome Project, most of the human genome was sequenced at 12× or greater coverage; that is, each base in the final sequence was present, on average, in 12 reads.

Whole genome shotgun sequencing for small (i.e., for example, 4,000 to 7,000 base pairs) genomes gave way to a broader application that benefited from pairwise end sequencing. Pair wise end sequencing performs sequencing from both ends of a read simultaneously, instead of a linear left-right process. Although sequencing both ends of the same fragment and keeping track of the paired data was more cumbersome than sequencing a single end of two distinct fragments, the knowledge that the two sequences were oriented in opposite directions and were about the length of a fragment apart from each other was valuable in reconstructing the sequence of the original target fragment. See, FIG. 17.

Paired end sequencing was first reported as part of the sequencing of the human HGPRT locus, although the use of paired ends was limited to closing gaps after the application of a traditional shotgun sequencing approach. Edwards et al., “Closure strategies for random DNA sequencing”. Methods: A Companion to Methods in Enzymology 3: 41-47 (1991). A theoretical description of a pure pairwise end sequencing strategy assuming fragments of constant length was also reported. Edwards et al., “Automated DNA sequencing of the human HPRT locus” Genomics 6:593-608 (1990). The method was improved by demonstrating that pair wise sequencing could be performed using fragments of varying sizes, thereby demonstrating a pairwise end-sequencing strategy would be possible on large genomic targets. Roach et al., “Pairwise end sequencing: a unified approach to genomic mapping and sequencing” Genomics 26:345-353 (1995). This strategy was successfully employed to sequence the genomes of Haemophilus influenzae, Drosophila melanogaster, and Homo sapiens. Fleischmann et al., “Whole-genome random sequencing and assembly of Haemophilus influenzae Rd.”. Science 269 (5223):496-512 (1995); and Adams et al., “The genome sequence of Drosophila melanogaster”. Science 287 (5461): 2185-2195 (2000).

To apply pair wise sequencing to high-molecular-weight DNA, the DNA can be sheared into random fragments, size-selected (i.e., for example, 2,10,50, and/or 150 kb), and cloned into an appropriate vector. The clones are then sequenced from both ends using the chain termination method yielding two short sequences. Each sequence is called an end-read, or read, wherein two reads from the same clone are referred to as mate pairs. Since the chain termination method usually can only produce reads between 500 and 1000 bases long, in all but the smallest clones, mate pairs will rarely overlap. The original DNA sequence is reconstructed from the numerous reads using sequence assembly software. First, overlapping reads are collected into longer composite sequences known as contigs. Contigs can be linked together into scaffolds by following connections between mate pairs. The distance between contigs can be inferred from the mate pair positions if the average fragment length of the library is known and has a narrow window of deviation. Conventional pair wise sequencing has disadvantages including but not limited to a need to improve reliability to correctly link regions, particularly for genomes with repeating regions.

Although shotgun sequencing was the most advanced technique for sequencing genomes from about 1995-2005, other technologies surfaced, called next-generation sequencing (supra). These technologies produce shorter reads (anywhere from 25-500 bp) but many hundreds of thousands or millions of reads are processed in a relatively short time (i.e., for example, within twenty-four hours). This results in high coverage, but the assembly process is much more computationally expensive. These technologies are vastly superior to chain termination shotgun sequencing due to the high volume of data and the relatively short time it takes to sequence a whole genome.

### VI. Genomic Assembly Computational Methods

Recent reports have described massively parallel technologies that can apparently produce DNA sequence information at a per-base cost that is ˜100,000-fold lower than a decade ago. Bentley et al., “Accurate whole human genome sequencing using reversible terminator chemistry” Nature 456:53-59 (2008); McKernan et al., “Reagents, methods, and libraries for bead-based sequencing” WO/2006/084132. Further, de novo genome assemblies using massively parallel sequence data have been reported for microbes with genomes up to 40 Mb. Zerbino et al., “Velvet: Algorithms for de novo short read assembly using de Bruijn graphs” Genome Res 18:821-829 (2008); Maccallum et al., “ALLPATHS 2: Small genomes assembled accurately and with high continuity from short paired reads” Genome Biol 10:R103 (2009); and Nowrousian et al., “De novo assembly of a 40 Mb eukaryotic genome from short sequence reads: Sordaria macrospora, a model organism for fungal morphogenesis” PLoS Genet 6:e1000891 ((2010). In one embodiment, the present invention contemplates a method for preparing short ‘reads’ (i.e., for example, ˜100 bps) that are compatible with high throughput sequencing.

In one embodiment, the ShaRc short ‘read’ fragments contemplated herein are processed by an algorithm capable of performing de novo assembly of large genomes (i.e., for example, a mammalian genome). For example, one such algorithm comprises an improved version of previously reported small genome assembly algorithms (i.e., for example, ALLPATHS). Maccallum et al., “ALLPATHS 2: Small genomes assembled accurately and with high continuity from short paired reads” Genome Biol 10:R103 (2009); and, Butler et al., ALLPATHS: De novo assembly of whole-genome shotgun microreads. Genome Res 18:810-820 (2008). The improved program is called ALLPATHS-LG (broadinstitute.orescience/programs/genome-biology/crd).

In one embodiment, the present invention contemplates a method for de novo assembly of a genome comprising short ‘reads’. In one embodiment, the short ‘read’ is approximately 100 base pairs. In one embodiment, the assembly method further comprises sequencing the short ‘reads’ using a high throughput sequencing platform. In one embodiment, the high throughput sequencing platform creates a database of massively parallel data. In one embodiment, the massively parallel data is processed by a genomic assembly algorithm (i.e., for example, ALLPATHS-LG). In one embodiment, the genome is a large genome. In one embodiment, the large genome is a human genome. In one embodiment, the large genome is a mouse genome. In one embodiment, the genome is a small genome. In one embodiment, the small genome is a microbial genome. In one embodiment, the algorithm uses massively parallel sequence data.

To demonstrate the effect of extra-long (i.e., fosIll) jumps on the long-range connectivity of de novo genome assemblies an Illumina-based ALLPATH-LG draft assemblies of the mouse genome was performed. Mouse genome Assembly 1, without fosIll's, had an N50 scaffold length of 2.6 Mb. Gnerre et al., “High-quality draft assemblies of mammalian genomes from massively parallel sequence data” Proc Natl Acad Sci USA 2010. Adding data from the first, smaller fosIll library (M1; 23-fold physical genome coverage) lengthened the N50 scaffold to 7.1 Mb.

Mouse genome Assembly 3 which used the second, more complex FosIll library instead (M2; 80-fold coverage) had an N50 scaffold length of 17.4 Mb, rivaling the long-range connectivity (16.9 Mb) of the capillary-sequencing-based draft assembly 4. Waterston et al., “Initial sequencing and comparative analysis of the mouse genome” Nature 2002, 420(6915):520-562. The scaffold accuracy, defined as the percentage of pairs of loci that were 100 kb apart in the assembly and had matching spacing and orientation in the reference genome, was about 99% for all four assemblies. Maccallum et al., “ALLPATHS 2: small genomes assembled accurately and with high continuity from short paired reads” Genome Biol 2009, 10(10):R103; and Table 8.

TABLE 8
Statistics for draft assemblies of the mouse genome

| Assembly | 1^{a} | 2^{b} | 3 | 4^{c} |
|---|---|---|---|---|
| Sequencing platform | Illumina | Illumina | Illumina | ABI3730 |
| XL jumps^{d} | None | FosIll | FosIll | Fosmid, |
| | | (M1) | (M2) | BAC |
| Physical coverage | N/A | 23x | 80x | 9.3x (Fosmid) |
| by XL jumps^{e} | | | | 13.7x (BAC) |
| N50 scaffold length | 2.6 | 7.2 | 17.5 | 16.9 |
| (Mb) | | | | |
| Scaffold accuracy^{f} | 99.0% | 99.2% | 99.1% | 99.1% |

^{a}Assembly based on paired end reads from ~180-bp fragment libraries and jumping constructs spanning up to 10 kb
^{b}Gnerre et al., “High-quality draft assemblies of mammalian genomes from massively parallel sequence data”. Proc Natl Acad Sci USA 2010
^{c}Waterston et al., “Initial sequencing and comparative analysis of the mouse genome” Nature 2002, 420(6915): 520-562
^{d}Extra long read pairs generated directly or indirectly from Fosmid or BAC libraries
^{d}Non-redundant set of unique jumps
^{f}Percentage of randomly chosen pairs of loci that spanned 100 kb in the assembly and had essentially the same spacing and orientation in the reference genome

Although it is not necessary to understand the mechanism of an invention, it is believed that the method contemplated herein provides a high quality genome assembly equivalent to capillary-based sequencing in terms of completeness, contiguity, connectivity, and accuracy. For example, the uncovered regions of a genome have many repetitive sequences, with segmental duplications remaining a particularly important challenge. The data presented herein indicate that it should be possible to generate high-quality draft assemblies of large genomes at ˜1,000-fold lower cost than a decade ago.

In contrast, some embodiments described herein comprising massively parallel sequencing data use a model where the library ‘read’ length is constant regardless of the desired sequencing coverage. See, Table 9.

TABLE 9
ShaRc De Novo Assembly Sequencing Model

| Libraries, | Fragment | Read length, | Sequence | |
|---|---|---|---|---|
| insert types* | size, bp | bases | coverage, x | Required |
| Fragment | 180^{†} | ≧100 | 45 | Yes |
| Short jump | 3,000 | ≧100 | 45 | Yes |
| | | preferable | | |
| Long jump | 6,800 | ≧100 | 5 | No^{‡} |
| | | preferable | | |
| Fosmid jump | 40,000 | ≧26 | 1 | No^{‡} |

*Inserts are sequenced from both ends, to provide the specified coverage.
^{†}More generally, the inserts for the fragment libraries should be equal to ~1.8 times the sequencing read length. In this way, the reads from the two ends overlap by ~20% and can be merged to create a single longer read. The current sequencing read length is ~100 bases.
^{‡}Long and Fosmid jumps are a recommended option to create greater continuity.

Although it is not necessary to understand the mechanism of an invention, it is believed that this ShaRc assembly model has specific advantages including, but not limited to; i) constructing only a few libraries; ii) reducing the laboratory burden and the amount of DNA required; iii) fragment library inserts that are short enough (i.e., for example, ˜100 bases) that the sequencing reads from each end overlap by ˜20% and can be merged to create a single longer read, but is should be noted that as read lengths increase, insert sizes should be ˜1.8 times the read length; iv) obtaining long-range connectivity by using “jumping libraries” in which the middle of the insert is removed. Collins et al., “Directional cloning of DNA fragments at a large distance from an initial probe: A circularization method” Proc Natl Acad Sci USA 81:6812-6816 (1984). It is further believed that removing part of the library insert overcomes the ˜1 kb sequencing limitation of the current technology.

In one embodiment, the present invention contemplates a method of genome assembly comprising an approximate 100-fold sequence coverage of the provided read pairs. This has specific advantages over capillary sequencing where the sequence coverage is typically only 8- to 10-fold. Although it is not necessary to understand the mechanism of an invention, it is believed that such sequence coverage compensates for the shorter reads and possible non-uniform coverage. Despite using a higher sequencing coverage threshold, the ShaRc assembly model incorporates the efficiencies of the per-base cost of massively parallel sequencing of being approximately 10,000-fold lower than the current cost of capillary sequencing. While sequencing coverage can be measured in different ways, Illumina sequencing coverage may be defined in terms of purity-filtered bases. Bentley et al., “Accurate whole human genome sequencing using reversible terminator chemistry” Nature 456:53-59 (2008), and Table 9.

TABLE 10
Representative ShaRc Sequencing Coverage Data

| | | No. of | DNA used, | Mean size, | Read | Sequence coverage, x | | | | | Physical |
|---|---|---|---|---|---|---|---|---|---|---|---|
| Species | Library type | libraries | μg | bp | length | All | PF | Aligned | Unique | Valid | coverage, x |
| Human | Fragment | 1 | 3 | 155 | 101 | 51.9 | 41.8 | 38.4 | 37.9 | 36.5 | 27.8 |
| | Short jump | 2 | 20 | 2,536 | 101 | 45.9 | 40.7 | 33.7 | 31.7 | 19.7 | 249.4 |
| | Fosmid jump | 2 | 20 | 35,295 | 76* | 5.3 | 4.0 | 3.0 | 0.4 | 0.3 | 49.5 |
| | Total | 5 | 43 | | | 103.1 | 86.5 | 75.1 | 70.0 | 56.5 | 326.7 |
| Mouse | Fragment | 1 | 3 | 168 | 101 | 58.6 | 53.1 | 49.6 | 46.6 | 45.3 | 37.6 |
| | Short jump | 3 | 20 | 2,209 | 101 | 48.0 | 40.7 | 35.1 | 32.0 | 19.9 | 219.1 |
| | Long jump | 5 | 50 | 7,532 | 26 | 13.5 | 9.3 | 9.2 | 5.5 | 2.9 | 408.3 |
| | Fosmid jump | 1 | 30 | 38,453 | 76 | 1.4 | 1.1 | 1.1 | 0.1 | 0.1 | 23.1 |
| | Total | 10 | 103 | | | 121.5 | 104.2 | 95.0 | 84.2 | 68.2 | 688.1 |

Library type: See Table 1.
DNA used: Amount of DNA used as input to library construction. For each genome and each library type, a single aliquot was used.
DNA source for human: Coriell Biorepository, NA12878.
DNA source for mouse: Jackson Laboratory C57/BL6J (stock 000664).
Size: Mean of observed fragment size distribution.
Read length: Number of bases sequenced. The exception is the long jump libraries prepared with the EcoP15I digestion, which yield 26 bases of genomic information; these inserts were sequenced to 36 bases and then trimmed to 26 bases.
Sequence coverage: All reads were used in the assembly, but we describe their properties here via a series of nested categories.
All: Total number of bases in reads, divided by genome size, assumed to be the reference size of 3.10 Gb for human and 2.73 Gb for mouse.
PF: Coverage by purity-filtered (PF) reads.
Aligned: Coverage by aligned PF reads.
Unique: Coverage by aligned PF reads, exclusive of duplicates, which were identified by concurrence of start and stop points of pairs on the reference.
Valid: Coverage by unique pairs for which the fragment length was within 5 SDs of the mean.
Physical coverage: Total coverage by valid pairs and the bases between them.
*Reads from one library had length 76, and those from the other had length 101.

In one embodiment, the present invention contemplates a method for making jumping libraries comprising, providing: (i) improving the recovery of high GC-content DNA fragments; (ii) using an Illumina protocol for short jumps (˜3 kb) (Bentley et al., “Accurate whole human genome sequencing using reversible terminator chemistry” Nature 456:53-59 (2008)); (iii) using a modified SOLiD sequencing platform for long jumps (=6 kb) involving circularization and EcoP15I digestion (McKernan et al., “Reagents, methods, and libraries for bead-based sequencing” WO/2006/084132; and Maccallum et al., “ALLPATHS 2: Small genomes assembled accurately and with high continuity from short paired reads” Genome Biol 10:R103); and (iv) for Fosmid jumps (˜40 kb) “ShARC” and “Fosill” are described herein.

In one embodiment, the present invention contemplates a method for improving mouse de novo genome assembly comprising adding fosIll read pairs. Although it is not necessary to understand the mechanism of an invention, it is believed that adding fosIll read pairs improves the N50 scaffold length of the ALLPATHS-LG assembly of the mouse genome from 2.6 to 17.5 Mb, rivaling the scaffold length of the capillary-sequencing-based draft assembly.

| Sequencing platform: | Illumina | Illumina | ABI3730 |
|---|---|---|---|
| XL (>10 kb) jumps: | None | FosIll | Fosmid, |
| | | | BAC |
| Physical coverage by | N/A | 80x | 9.3x (Fosmid) |
| XL jumps: | | | 13.7 (BAC) |
| N50 scaffold length: | 2.6 Mb | 17.5 Mb | 16.9 Mb |
| Scaffold accuracy: | 99.0% | 99.1% | 99.1% |

### VIII. Kits

In another embodiment, the present invention contemplates kits for the practice of the methods of this invention. The kits preferably include one or more containers containing a fosmid vector capable of performing methods contemplated herein. The kit can optionally include a fosmid cloning vector comprising a cloning site comprising a plurality of polylinker sites, wherein said polylinker sites are flanked by universal primer sequences and nicking endonuclease sites. The kit can optionally include a fosmid cloning vector comprising a cloning site, wherein said cloning site is flanked by universal sequences and Illumina sequencing primer binding sites. The kit can optionally include enzymes and reagents for circularization (i.e., for example, a DNA ligase) and exonuclease treatment (i.e., for example, a plasmid-safe DNA exonuclease). The kit can optionally include an endonuclease capable of nicking the endonuclease site. The kit can optionally include enzymes and reagents for performing inverse polymerase chain reaction. For example, the kit can optionally include enzymes such as DNA polymerase, Taq polymerase, PCR primers and/or restriction enzymes. The kits may also optionally include appropriate systems (e.g. opaque containers) or stabilizers (e.g. antioxidants) to prevent degradation of the reagents by light or other adverse conditions.

The kits may optionally include instructional materials containing directions (i.e., protocols) providing for the use of the compositions and/or reagents in the present invention. In one embodiment, the kit further comprises instructions for incorporating a genomic nucleic acid sequence into the fosmid cloning vector to create a fosmid library. In one embodiment, the kit further comprises instructions for using the fosmid library with next-generation sequencing platforms. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXPERIMENTAL

### Example I

### Construction of FosIll-1 and pFosIll-2

pFOS1 (Kim et al., “Stable propagation of cosmid sized human DNA inserts in an F factor based vector” Nucleic Acids Research, 20:1083-1085 (1992)) was purchased as a frozen bacterial stock in E. coli pop2136 from New England Biolabs.

Standard procedures for recombinant DNA experiments were used. Sambrook et al., Molecular Cloning, Cold Spring Harbor (1989) to modify pFOS1. To generate pFosIll-1, the following synthetic sequence comprising a Kanamycin resistance cassette flanked by polylinker, nicking endonuclease cloned in the EcoRV site of pUC57 was commercially purchased:

(SEQ ID NO: 5)
AAGCTTAATGATACGGCGACCACCGACACTGCTGAGGACACTCTTTCCCT
ACACGACGCTCTTCCGATCTCCACGTGCATGCTGGATCCATCATGAACAA
TAAAACTGTCTGCTTACATAAACAGTAATACAAGGGGTGTTATGAGCCAT
ATTCAACGGGAAACGTCTTGCTCGAGGCCGCGATTAAATTCCAACATGGA
TGCTGATTTATATGGGTATAAATGGGCTCGCGATAATGTCGGGCAATCAG
GTGCGACAATCTATCGATTGTATGGGAAGCCCGATGCGCCAGAGTTGTTT
CTGAAACATGGCAAAGGTAGCGTTGCCAATGATGTTACAGATGAGATGGT
CAGACTAAACTGGCTGACGGAATTTATGCCTCTTCCGACCATCAAGCATT
TTATCCGTACTCCTGATGATGCATGGTTACTCACCACTGCGATCCCCGGG
AAAACAGCATTCCAGGTATTAGAAGAATATCCTGATTCAGGTGAAAATAT
TGTTGATGCGCTGGCAGTGTTCCTGCGCCGGTTGCATTCGATTCCTGTTT
GTAATTGTCCTTTTAACAGCGATCGCGTATTTCGTCTCGCTCAGGCGCAA
TCACGAATGAATAACGGTTTGGTTGATGCGAGTGATTTTGATGACGAGCG
TAATGGCTGGCCTGTTGAACAAGTCTGGAAAGAAATGCATAAACTTTTGC
CATTCTCACCGGATTCAGTCGTCACTCATGGTGATTTCTCACTTGATAAC
CTTATTTTTGACGAGGGGAAATTAATAGGTTGTATTGATGTTGGACGAGT
CGGAATCGCAGACCGATACCAGGATCTTGCCATCCTATGGAACTGCCTCG
GTGAGTTTTCTCCTTCATTACAGAAACGGCTTTTTCAAAAATATGGTATT
GATAATCCTGATATGAATAAATTGCAGTTTCATTTGATGCTCGATGAGTT
TTTCTAATCAGAATTGGTTAATTAGCCCGCCTAATGAGCGGGCTTTTTTT
TGGATCCAGCATGCACGTGGAGATCGGAAGAGCGGTTCAGCAGGAATGCC
GAGACCGATCCTCAGCAGTGTCGTATGCCGTCTTCTGCTTGAGATCT

The fragment excized by digestion with HindIII and BgIII was inserted into pFOS1 that had been digested with HindIII and BamHI. To generate pFosIll-2, pFosIll-1 was digested with BamHI, and larger of the two resulting fragments re-cicularized and cloned, resulting in simplified fosill cloning vector lacking the kanamycin stuffer fragment.

### Example II

### Construction of Microbial Clonal Libraries

Construction of a fosmid library may be performed according to the procedure for double cos cosmids. Bates P., Methods in Enzymol. 153:82-94 (1987). To generate two arms, the plasmids may be completely digested with AatH (pFOS1, pFosIll-1, pFosIll-2 and Lawrist 16) or XbaI (Supercos), dephosphorylated by alkaline phosphatase, and digested with BamHI or Eco72I (pFosIll-1 and pFosIll-2). The arms are ligated to isolated DNA that has been partially digested with MboI (e.g., pFOS-1, Supercos) or randomly sheared and end repaired (pFosIll-1 and pFosIll-2) and size-selected on a pulsed-field gel to ˜30-45 kb. The ligated DNA can be in vitro packaged by using Gigapak Gold® packaging system (Stratagene) or other commercial sources of lambda packaging extract. The cosmid or fosmid particles were titered by using an aliquot to transfect E. coli strain DH10B followed by spreading on a selective plate (chloramphenicol). The rest of the packaged fosmid particles were then mass-transfected into E. coli DH10B cells followed by overnight growth at 30° C. in liquid media that selects for chloramphenicol-resistance. The culture was stopped at an OD600=1 followed by a maxiprep of the entire library of ˜35-50 kb fosmid circles.

### Example III

### Bridge Amplification Nucleic Acid Sequencing

This example provides a description of one method to use a bridge amplification sequencing platform on the fosIll jumping libraries described herein. One having ordinary skill in the art would recognize that this description can be modified.

### Preparation of Flowcells

Glass 8-channel flow cells (Silex Microsystems, Sweden) can be thoroughly washed and then coated for 90 min at 20° C. with 2% acrylamide containing ˜3.9 mg/ml N-(5-bromoacetamidylpentyl) acrylamide, 0.85 mg/ml tetramethylethylenediamine (TEMED) and 0.48 mg/ml potassium persulfate (K2S208). Flow cell channels can be rinsed thoroughly before further use. The coated surface may be then functionalised by reaction for 1 hour at 50° C. with a mixture containing 0.5 μM each of two priming oligonucleotides in 10 mM potassium phosphate buffer pH 7. Grafted flow cells can be stored in 5×SSC until required.

### Cluster Creation for Single Read Experiments

Cluster creation may be carried out using an Illumina Cluster Station. To obtain single stranded templates, adapted DNA may be first denatured in NaOH (to a final concentration of 0.1M) and subsequently diluted in cold (4° C.) hybridisation buffer (5×SSC+0.05% Tween 20) to working concentrations of 2-4 pM, depending on the desired cluster density/tile. 85 μl of each sample can be primed through each lane of a flow cell at 96° C. (60 μl/min). The temperature may be then slowly decreased to 40° C. at a rate of 0.05° C./sec to enable annealing to complementary adapter oligonucleotides immobilised on the flow cell surface (i.e., for example, oligo ‘A’: 5′-PSTTTTTTTTTT-(diol)3-AATGATACGGCGACCACCGA-3′; oligo ‘B’: 5′-PSTTTTTTTTTTCAAGCAGAA GACGGCATACGA-3′). Hybridised template strands can be extended using Taq polymerase to generate their surface-bound complement. The samples can be then denatured using formamide to remove the initial seeded template. The remaining single stranded copy may be the starting point for cluster creation. Clusters can be amplified under isothermal conditions at 60° C. for 35 cycles using Bst polymerase for extension and formamide for denaturation during each cycle. Clusters can be may be washed with storage buffer (5×SSC) and either stored at 4° C. or used directly.

### Cluster Creation for Paired Read Experiments

Paired read flowcells contained the two oligonucleotides: oligo ‘C’ 5′-PS-TTTTTTTTTTAATGATACGGCGACCACCGAGAUCTACAC-3′ (U=2-deoxyuridine) and

oligo ‘D’: 5′-PS-TTTTTTTTTTCAAGCAGAAGACGGCATACGAGoxoAT-3 (Goxo=8-oxoguanine) immobilised on the surface in a ratio C:D=1:1. Other than the use of a paired-end specific library, cluster creation may be the same as described above.

### Processing of Clusters for Single Read Experiments

Linearisation of surface-immobilised complimentary oligonucleotide ‘A’ may be achieved by incubation with linearization mix (100 mM sodium periodate, 10 mM 3-aminopropan-1-ol, 20 mM Tris pH 8.0, 50% v/v formamide) for 20 minutes at 20° C. followed by a water wash. All exposed 3′-OH termini of DNA, either from the extended template or unextended surface oligonucleotides can be blocked by dideoxy chain termination using a terminal transferase and ddNTPs. Linearised and blocked clusters can be denatured with 0.1M NaOH prior to hybridisation of the sequencing primer. Processed flowcells can be transferred to the Illumina Genome Analyser for sequencing.

### Processing of Clusters for Paired Read Experiments

For read 1, linearisation of surface immobilised oligonucleotide ‘C’ to retain strand 1 of each cluster may be achieved by incubation with USER enzyme (as shown above). After blocking, clusters can be denatured with 0.1 M NaOH prior to hybridisation of the read 1 specific sequencing primer (5′-ACACTCTTTCCCTACACGACGCTCTTCCGATCT-3′). Processed flowcells can be transferred to the Illumina Genome Analyser for sequencing.

Following the successful completion of sequencing of read 1 on the Genome Analyser, flowcells remained mounted and can be automatically prepared for read 2 in situ using the Illumina Paired End module (according to operating manual). Clusters can be denatured with 0.1 M NaOH to remove the products of read 1. Clusters can be 3′-dephosphorylated using T4 polynucleotide kinase, and the strand that had been linearised as part of the read 1 preparation may be re-synthesized isothermally as previously described for cluster creation. Linearisation to remove strand 1 of the resynthesised clusters may be achieved by the excision of 8-oxoguanine from oligo ‘D’ using Fpg (foimamidopyrimidine DNA glycosylase, New England Biolabs). Linearised and blocked clusters can be denatured with 0.1M NaOH prior to hybridisation of the read 2 specific sequencing primer (5′-CGGTCTCGGCATTCCTGCTGAACCGCTCTTCCGA TCT-3′).

### Sequencing on the Genome Analyser

All sequencing runs can be performed as described in the Illumina Genome Analyser operating manual. Flowcells can be sequenced using standard recipes (see User Guide) in order to generate 75+ base single and paired reads. Typically a single read run producing 1-2 Gb of PF data required 72 hours; paired read runs required approximately 150 hours including the time taken for automated preparation of the template for the second read.

### Image Analysis

An image analysis program (i.e., for example, Firecrest®) first identifies the position of the DNA clusters on the images taken from the first sequencing cycle. Each initial image may be band-pass filtered to remove background fluorescence and large-scale structure on the image, as well as enhance the signal-to-noise. Cluster positions can be identified from a search for local maxima on the filtered image. Because of the finite accuracy of the movements of the motion stage, images taken at different sequencing cycles have random translational offsets with respect to each other. Furthermore, images taken in different frequency channels have different optical paths and wavelengths and experience further, albeit smaller, translations and scale transformations. In order to correct for the image shifts and scalings, the cluster positions that can be extracted from the four images taken in the first cycle can be super-imposed to construct a ‘reference image’ containing all detected clusters. Transformations of the image coordinates to later cycles can be then obtained from a cross-correlation of the images in later cycles to the reference image. In this way, a set of four intensity measurements can be obtained for each cluster and sequencing cycle. These series of intensities for each cluster are analogous to the intensity traces from Sanger sequencing. In addition to estimates of the intensities, the image analysis also extracts an estimate of the local noise or image background dispersion around the cluster for each image.

### Base Calling

The signals detected for the four different dye-labelled dNTPs are not independent, as the emission spectra of their dyes and the transmission and detection frequency windows may overlap between nucleotides. The relative intensities and cross-talk are described by a frequency cross-talk matrix, which characterises the intensity response of the system to each nucleotide. A method to auto-calibrate this matrix uses the intensity traces and applies a correction to the extracted intensities. Because the rates of phasing and prephasing can be small and consistent, the resulting intensity dissipation into different frequency cycles may be also small and the accumulated loss roughly linear. Therefore, phasing and prephasing rates can be estimated by measuring the build-up of correlated signal between different cycles over early sequencing cycles. From these rates the expected correlation of signals can be derived for each cycle and de-correlated them. The end result of these computations may be a set of matrix corrected, phasing-corrected intensity values for each cluster, from whichever of the four channels gave the highest value at a given cycle to be the base call for that cycle.

### Purity Filtering

In order to discriminate between good reads without errors and reads derived from mixed clusters that overlap their nearest neighbours, a measure of signal purity can be defined at a given cycle by taking the brightest of the four corrected intensities at that cycle as a fraction of the sum of the brightest and next brightest intensities. All reads may be discarded whose corrected brightest intensity in any of the first 12 sequencing cycles may be less than 60% of the sum of brightest intensity and the next brightest. This criterion can provide reasonable discrimination between good and bad data. Depending on the loading density, typically between 50% and 70% of the raw reads can be retained. All figures quoted for accuracy and yield per flowcell refer only to this purity-filtered subset of the raw data.

### Quality Scoring of Bases

A base caller program (i.e., for example, Bustard®) provides a first estimate of the uncertainty of the base call. This is computed by propagating the noise estimates from the image analysis and integrating the resulting likelihood functions to obtain probability estimates for each of the four possible base calls. The probability estimates are transformed to scores by first converting to a log-odds scale via the formula Q=10 log 10(p(X)/(1−p(X)), where p(X) for A,C,G,T is the estimated probability of the base call being X, and then rounding to integers. This scoring scheme can be thought of as a generalization of the scoring scheme made popular by the Phred base caller10 (Q−10 log 10(perror), where perror is the probability of an incorrect base), in that the highest of the four scores—that of the called base—is asymptotic to the Phred score, but the scheme also enables meaningful integer scores to be assigned to the three non-called bases.

This initial base quality estimate is refined by an implementation of the Phred algorithm, taking as predictors the initial confidence score for the called base together with the sequencing cycle, the purity of the called base and the minimum purity over the first 12 bases of the read. The sequence data for each flowcell lane in an experiment can be used as a training set for the same lane, with the alignment to the reference being used to determine whether or not a base is correct. Since a lane may produce several hundred million base pairs, each lane contains enough data to serve as a reasonable training set. After this procedure, the observed error rate for each base closely matches the error rate implied by the adjusted quality score.

### PhageAlign® Alignment

PhageAlign® is a program for exhaustive alignment of reads of length k against a known reference. Both strands of the reference sequence are split into overlapping kmers which are then sorted lexicographically. The reads are also sorted and then compared to each genomic k-mer in turn allowing any number of substitution errors.

Commonality of prefixes between lexicographically adjacent prefixes is exploited to minimise the number of base to base comparisons required, however its exhaustive nature renders it too slow for high-throughput alignment of datasets where either the number of reads or the size of the reference genome is large. The principal use of PhageAlign® is to enable an accurate measure of raw read error by allowing even noisy reads to participate in the error rate calculation, provided the aligner is able to find a unique best match for them in the reference.

### ELAND® Alignment

In order to remap the sequence reads to a large reference genome, a fast short-read alignment program called ELAND® can be used. The first few bases (32 bp default, or the entire read length for reads shorter than this) are aligned to the genome allowing up to two substitution differences to obtain a set of candidate match positions for each read in the reference. Sequence from each of these positions is then used to extend the candidate alignments along the full length of each read. Finally base quality values are used to choose, where possible, the most probable of the candidate alignments. For paired reads, a set of candidate alignments is obtained for each of the two reads as described above. Read pairs having a unique alignment of each read are first used to determine the nominal strand orientation and insert size distribution of the sample then, on a second pass, this information is used to resolve repeats and determine the anomalously paired reads that are possible indicators of structural variation.

### ELAND SNP Calling

For allele calling, read pairs may be used that have alignments that can be correctly oriented and that indicated a template insert size of within 3 standard deviations of the sample median. A paired alignment score of approximately >6 (indicating the quality of the paired mapping) may also be useful. The basecalls and their associated quality values can be sent to a Bayesian allele caller, which produced one or two allele calls and scores for each position in the genome. At each position, the allele caller computes log 10 p(observed bases |no “A”s are present) and similarly for C,G and T. The highest two scores are then normalized by subtracting the third highest, thus obtaining log-odds scores for the two most probable alleles for which an increment in score of 3 approximately corresponds to an increase in coverage of a single base of Phred quality 30. SNPs can be called where a non-reference base allele may be observed, the allele call score may be ≧10, and the depth at this position may be no greater than three times the chromosomal mean. For heterozygous calls, both alleles can have an allele-call score ≧10 and the ratio of their scores to be ≦3. For the reduced depth analysis an allele-call score is >6. SNP calls maybe excluded that are within 15 bp of an apparent small indel.

### ELAND® Structural Variant Detection

Hierarchical clustering of anomalous readpairs may be used to identify groupings of five or more readpairs that had a similar size and position. Read pairs can be defined as anomalous if they had high-confidence alignments of each individual read that nevertheless can be either incorrectly oriented or implied an insert size of at least 3 standard deviations outside the sample median. These groupings can be combined with other information such as depth changes, alignability and gaps in expected coverage, and a ranking system may be applied. Higher (positive) ranks can be assigned where the event supporting evidence may be seen; negative ranks where used for regions where it would be difficult to call variants, such as the centromere, or where contradictory evidence may be seen.

Some structural variants can be characterised using local de novo assembly. For example, high quality (23 of the first 25 bases had ≧Q20) anomalous pairs, singletons and their non-aligning partner may be selected within the region of interest for an attempted assembly using Velvet®. Contigs can be aligned back to the chromosome using BLAST, in order to look for discontinuities in the alignment, indicating breakpoints.

### MAQ Alignment

MAQ first searches for the ungapped match with lowest mismatch score, defined as the sum of qualities at mismatching bases. To speed up the alignment, MAQ only considers positions that have 2 or fewer mismatches in the first 28 bp. Sequences that fail to reach a mismatch score threshold but whose read pair is mapped are searched with a gapped alignment algorithm in the regions defined by the read pair. To evaluate the reliability of alignments, MAQ assigns each alignment a Phred scaled quality score which measures the probability that the true alignment is not the one found by MAQ. MAQ always reports a single alignment, and if a read can be aligned equally well to multiple positions, MAQ will randomly pick one position and give it a mapping quality zero.

MAQ fully utilizes the read-pair information of paired reads. It is able to use this information to correct wrong alignments, to add confidence to correct alignments, and to accurately map a read to repetitive sequences if its mate is confidently aligned. With paired-end reads, MAQ also finds short insertions/deletions (indels) from the gapped alignment described above.

### Calculation of Mapped Read Depth and Distribution

After aligning the data to the reference sequence, the depth of mapped reads may be sampled at every 50th position. Then all the positions from this sample can be discarded where the reference is not unique on the scale of the read length (as determined by mapping the reference to itself). Comparison with the Poisson distribution having the same mean may shows that there is some extra variance or overdispersion relative to the theoretical minimum.

At each of the unique positions the GC content of the reference in a surrounding window of length twice the read length may be calculated. This gives an estimate of the GC content of all the reads that could have overlapped that position. Th positions may then be binned by GC content, and within each bin calculated the mean depth and the 10th and 90th centiles of both the depth and a Poisson distribution with the same mean.

### MAQ SNP Calling

MAQ produces a consensus genotype sequence from the alignment. The consensus sequence is inferred from a Bayesian statistical model and each consensus genotype is associated with a Phred quality which measures the probability that the consensus genotype is incorrect. Potential SNPs are detected by comparing the consensus sequence to the reference and are further filtered by a set of predefined rules. These rules are:
i) discard SNPs within the 3 bp flanking region around a potential indel;
ii) discard SNPs covered by three or fewer reads;
iii) discard SNPs covered by no read with a mapping quality higher than 60;
iv) in any 10 bp window, if there are 3 or more SNPs, discard them all;
v) discard SNPs with consensus quality smaller than 20; and
vi) discard a SNP if a base with consensus quality lower than 20 occurs within 3 bp on either side of the target SNP.

### MAQ Small Indel Detection

MAQ regards an indel is reliable, if at least three reads contain the exact indel (identical position and indel size). MAQ only keeps one most evident indel in any 10 bp window because close indels may indicate alignment artefacts.

### Genome-Wide De Novo Assembly of Unaligned Reads

De novo assembly may be performed using Velvet® and unaligned read pairs. The read pairs can be duplicate filtered and then further quality filtered to obtain the best reads, to allow for computer memory limitations. Velvet® 0.5.05 may be used with a hash length of 23, a coverage cutoff of 5 and a maximum insert length of 350. The contigs can be then filtered to ensure a minimum length of 100 bases.

### MAQ Structural Variant Detection

Anomalous read pairs with a mapping quality of at least 20 can be ordered first by start position, then two anomalous pairs can be allocated to the same cluster if they can be overlapping and if their end positions can be not further apart than a given threshold (mean of the insert lengths plus three times their standard deviation). This procedure may be followed by merging neighbouring overlapping clusters. To obtain a final set of putative deletions, these candidate clusters can be filtered for the number of read pairs per cluster (at least 5), for distance between leftmost and rightmost forward reads (distance is less than a given threshold, i.e. mean of the insert lengths plus three times their standard deviation), similarly for distance between leftmost and rightmost reverse reads and for deletion size (greater than a given threshold, i.e. mean of the insert lengths plus three times their standard deviation); also read depth and repeat structures can be considered. Putative deletions greater than 100 kb can be removed. Mapped read depth may be used to infer copy-number variants between the sample (NA07340) and reference sequences. At any given sequence position the depth of reads aligned by Maq is expected to be Poisson distributed, with mean determined by the copy numbers of both the sample and the reference at that position, the mean overall depth of coverage, and any GC or other biases. A Hidden Markov model can be constructed with hidden states representing copy number differences between sample and reference and an emission or observed variable representing the mapped depth accounting for GC content. Standard HMM methods can be then applied to infer the most probable sequence of copy-number states in the sample given the depth data.

### Resembl®

Resembl® is an extended version of Ensembl® and allows storage, query and viewing of Illumina resequencing data in a genomic context. The data and re-sequencing datasets from ELAND®-based alignment data can be loaded into Resembl® databases and Resembl® websites can be used for interactive data mining and QC content. The Resembl® back-end database may be designed to allow storage and retrieval of the large amounts of re-sequencing data in an efficient way. It supports paired-end alignments at high coverage, as well as per-base and summary-type data on coverage and alignability. Parsing scripts can be written to pre-process sort.txt files from the build process and adapted to run on a Linux clustered environment. Import scripts can be written for very large-scale data loading. A system of clustered indexes may be developed to allow fast retrieval of data from multi billion record tables.

The website extensions to the Ensembl® browser allow visualization of re-sequencing data in a genomic context and support easy navigation back to the raw data. Extensions can be made to Ensembl's Karyotype, Map and Contig View, and a new view (called ReadView) may be added for closer examination of reads. Resembl websites can be set-up for browsing the X and whole human genome paired-end alignments, SNPs and structural variations, as well as coverage and alignability graphical plots.

Paired alignments are categorised into Regular and Anomalous (i.e. anomalousgapped, misoriented, chimeras, singletons) and displayed in tracks, accordingly. Suitable colour coding is used to distinguish the different types of variation. The displayed paired alignments are also filtered according to the rules used during the pipeline analysis. Candidate structural variants are highlighted by peaks in a graphical plot within ContigView. SNPs produced from the pipeline are loaded in Resembl® as a user track. All the extensions can be seamlessly integrated within Ensembl®, and can be flexibly implemented as a plugin.

### Example IV

### Preparation of fosIll Libraries: Overview

The Fosill protocol for making 40 kb jumping libraries may be split into 4 parts.
Part A: pFosill Vector Arm Preparation
Part B: Genomic Insert DNA Preparation
Part C: Fosmid Preparation
Part D: Illumina Library Preparation

pfosIll(ΔKan) is a modified pFOS1 plasmid that has Illumina paired end adapter sequences and two nicking endonuclease sites within the vector. See, FIG. 7. Digestion of pfosIll(ΔKan) with AatII/Eco72I releases the 2 “arms” needed for the fosmid production stage of the protocol. fosmid libraries may be made by ligating the pfosill(ΔKan) arms to size selected insert genomic DNA (˜40 kb), packaging the ligation using phage lambda extract and transforming E. coli. 40 kb plasmid DNA can then be isolated from E. coli. fosmid libraries are subsequently converted into Illumina libraries by initiating a controlled nick translation reaction from two nicking restriction sites within the vector sequence into the ends of the insert DNA. The nick translation reaction is stopped and the nicks are cleaved using S1 nuclease. Recircularization of the DNA followed by PCR results in amplification of an Illumina sequencable library.

### Example V

### Part A: pFosill-2 Vector Arm Preparation

pfosIll-2 (ΔKan) (hereinafter pfosIll(ΔKan)) is digested with AatII (Fermentas) and Eco72I (Fermentas), to produce 2 arms (7.3 kb and 2.6 kb) which are then dephosphorylated using Calf Intestinal Phosphatase (NEB). Traditionally, fosmid vector arms are not dephosphorylated.

Materials for pfosIll(ΔKan) Plasmid Prep and for Making Vector Arms

Lab Instruments
Dark Reader Transilluminator (Clare Chemical Research)
Qubit Fluorometer (Invitrogen, Q32857)

Store at −80° C.
glycerol stock of pFosill(ΔKan) in Stbl2 cells (Invitrogen) Store at −20° C.
50 mg/ml carbenicillin
25 mg/ml chloramphenicol
Plasmid-Safe™ ATP-Dependent DNase 10 U/ul (Epicentre, E3110K)
25 mM ATP, supplied with plasmid safe DNase) (Epicentre, E3110K)
10 mM ATP 10 U/ul (NEB, P0756S)
AatII 10 U/ul (Fermentas, ER0992)
Eco72I 10 U/ul (Fermentas, ER0361)
BglII 10 U/ul (NEB, R0144S)
NotI 10 U/ul (NEB, R0189S)
PstI 20 U/ul (NEB, R0140S)
XmaI 10 U/ul (NEB, R0180S)
BSA (100×) (NEB, B90015)
10×NEB Buffer 4 (NEB, 870045)
10×NEB Buffer 3 (NEB, B70035)
10×NEB Buffer 2 (NEB, B7002S)
Sybr Green I (Invitrogen, 5-7563)

Store at 4° C.
LB agar plates with 15 ug/ml chloramphenicol and 100 ug/ml carbenicillin
NEB 2-Log DNA Ladder (0.1-10.0 kb) (NEB, N3200L)
AmPure XP beads (Beckman Coulter (Agencourt), A63881)

Store at room temperature
QIAfilter Plasmid Purification Mega Kit (Qiagen, 12281)
LB media
Glycerol (Sigma, G5516)
Quant-iT dsDNA BR assay kit (_{0.2-100ng}) (Invitrogen, Q32850)
Qubit Assay Tubes 0.5 ml (Invitrogen, Q32856)
Agarose (IBI scientific, IB70042)
5× loading dye (Bio-Rad, 161-0767)
1×TAE
low TE (pH 8.0, 10 mM Tris, 0.1 mM EDTA)
   Step 1. pfosIll(ΔKan) Plasmid Preparation

1a: Grow Colonies from Glycerol Stock

Streak the glycerol stock, containing Stbl2 cells transformed with pFosill(ΔKan), onto a LB agar plate (15 ug/ml chloramphenicol and 100 ug/ml carbenicillin) and incubate overnight at 30° C. The next day single colonies should be present from which cultures for plasmid preps can be started.

1B: Large Scale Plasmid Prep

Pick a single colony from the streaked plate and inoculate a starter culture of 4 mls LB plus 100 ug/ml carbenicillin and 15 ug/ml chloramphenicol. Incubate at 30° C. with shaking at 250 rpm for about 6 hours. Transfer the starter culture to 500 mls of LB plus 100 ug/ml carbenicillin and 15 ug/ml chloramphenicol, grow overnight at 30° C. with shaking at 250 rpm. Keep a couple ml of the LB plus 100 ug/ml carbenicillin and 15 ug/ml chloramphenicol to use as a blank for measuring cell density the next morning.

Measure the OD₆₀₀ using a spectrophotometer the next morning. Want cells to have an OD₆₀₀ of around 1. Make a glycerol stock of the culture as follows. Add 500 ul of cells to 500 ul of 50% glycerol, mix and store at −80° C. pFosill(ΔKan) plasmid preps were made using a QIAfilter Plasmid Purification Mega Kit (Qiagen, 12281), according to the manufacturer's instructions.

Briefly:
1. Harvest the bacterial cells by centrifugation at 6000×g for 15 min at 4° C. The cells can be stored at −20° C. or processed to make the pFosill(ΔKan) plasmid prep.
2. Screw the QIAfilter Mega-Giga Cartridge onto a 45 mm-neck glass bottle and connect it to a vacuum source.
3. Resuspend the bacterial pellet in 50 ml of Buffer P1.
4. Add 50 ml of Buffer P2, mix thoroughly by vigorously inverting 4-6 times, and incubate at room temperature for 5 min.
5. Add 50 ml 1 chilled Buffer P3 and mix thoroughly by vigorously inverting 4-6 times. Mix well until white, fluffy material has formed and the lysate is no longer viscous. Proceed directly to step 6. Do not incubate on ice.
6. Pour the lysate into the QIAfilter Mega-Giga Cartridge and incubate at room temperature for 10 min.
7. Switch on the vacuum source. After all liquid has been pulled through, switch off the vacuum source. Leave the QIAfilter Cartridge attached.
8. Add 50 ml Buffer FWB2 to the QIAfilter Cartridge and gently stir the precipitate using a sterile spatula. Switch on the vacuum source until the liquid has been pulled through completely.
9. Equilibrate a QIAGEN-tip 2500 by applying 35 nil Buffer QBT, and allow the column to empty by gravity flow.
10. Apply the filtered lysate from step 10 onto the QIAGEN-tip and allow it to enter the resin by gravity flow.
11. Wash the QIAGEN-tip with a total of 200 ml Buffer QC
12. Elute DNA with 35 ml Buffer QF.
13. Precipitate DNA by adding 24.5 ml room-temperature isopropanol to the eluted DNA. Mix and centrifuge immediately at >15,000×g for 30 min at 4° C. Carefully decant the supernatant.
14. Wash DNA pellet with 7 ml of room-temperature 70% ethanol, and centrifuge at ≧15,000×g for 10 min. Carefully decant the supernatant without disturbing the pellet.
15. Air-dry the pellet for 10-20 min, and resuspend the DNA in 1 ml low TE (pH8.0, 10 mM Tris, 0.1 mM EDTA).

1C: DNA Concentration of Plasmid Preparation

The DNA concentration is measured using Quant-iT dsDNA BR assay kit on the Qubit Fluorometer according to the manufacturer's instructions. Typically 750-1000 ug of plasmid is recovered. The plasmid is aliquoted and kept at 4° C. for near term use or at −20° C. for long term storage.

1D: Restriction Digests of pfosill(ΔKan)

Restriction digests were carried out on pfosIll(ΔKan) to confirm that there were no gross rearrangements. 200 ng aliquots of pfosIll(ΔKan) were digested with the following enzymes:

Restriction Enzymes

Eco72I-AatII
BglII-NotI
PstI-BglII
XmaI-AatII
BglII-Eco72I

All restriction digests were incubated for 1 hour at 37° C. using the appropriate supplied buffers and should give product sizes as indicated below.

| | Restriction | | Digest | Length |
|---|---|---|---|---|
| # | Enzymes | Buffer | Coordinates | (bp) |
| 1 | Eco72I-AatII | Tango | 2609-2 | 7299 |
| 2 | | (Fermentas) | 3-2584 | 2582 |
| 3 | | | 2585-2608 | 24 |
| 1 | BglII-NofI | NEB 3 | 6659-2482 | 5729 |
| 2 | | | 4592-6658 | 2067 |
| 3 | | | 2714-4591 | 1878 |
| 4 | | | 2483-2713 | 231 |
| 1 | PstI-BglII | NEB 2 | 7012-4591 | 7485 |
| 2 | | | 5471-6658 | 1188 |
| 3 | | | 4592-5470 | 879 |
| 4 | | | 6659-7011 | 353 |
| 1 | XmaI-AatII | NEB 4 | 4937-2 | 4971 |
| 2 | | | 3-2719 | 2717 |
| 3 | | | 2722-4938 | 2217 |
| 1 | BglII-Eco72I | NEB 2 | 6659-2584 | 5831 |
| 2 | | | 4592-6658 | 2067 |
| 3 | | | 2609-4591 | 1983 |
| 4 | | | 2585-2608 | 24 |

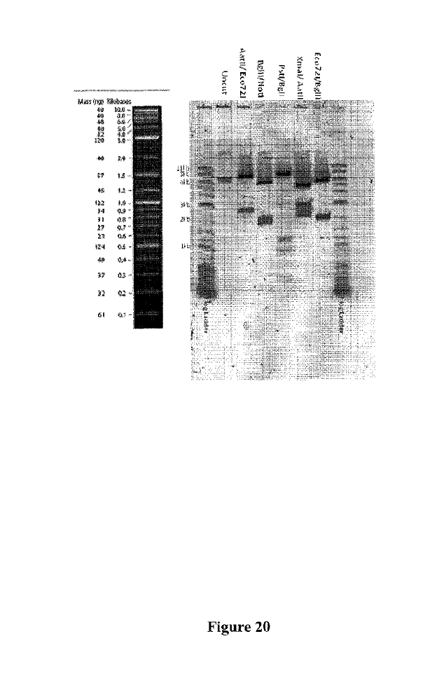

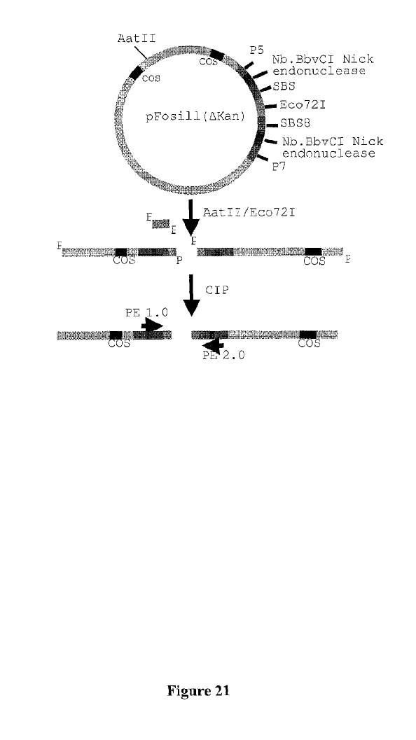

Run 200 ng uncut pfosIll(ΔKan), the pfosIll(ΔKan) digests and 2 log marker (NEB) on a 0.7% agarose gel in 1×TAE at 80V for 4 hrs and stain with Sybr Green I (15 ul in 150 mls water) for 45 min. See FIG. 20.

1E: Plasmid Safe DNase

A background DNA smear was present on the pfosIll(ΔKan) restriction digests. This could be contaminating E. coli genomic DNA. To remove this from the plasmid prep, the DNA was treated with Plasmid-Safe™ ATP-Dependent DNase.

Used 1.5 U Plasmid-Safe™ ATP-Dependent DNase for 1 ug plasmid DNA.

| | ul |
|---|---|
| pfosIll-ΔKan (200 ug) | x |
| 10X Plasmid Safe DNase Buffer | 50 |
| Water | y |
| 25 mM ATP | 20 |
| Plasmid Safe DNase (10 U/ul) | 30 |
| Final volume | 500 |

Incubate for 30 min at 37° C. and then inactivate at 70° C. for 30 min Clean up the plasmid safe DNase treated pFosill(ΔKan) using Agencourt AMPure XP beads (Beckman Coulter) as follows:
A: Split the plasmid safe DNase treated DNA into two 1.5 ml Eppendorf tubes.
B: Shake the AMPure XP bottle to resuspend any magnetic particles that may have settled. Add 1.8× vol (450 ul) of AMPure XP beads to each tube.
C: Pipette up and down 10 times and incubate for 5 minutes at room temperature.
D: Hold against a magnet for at least 2 minutes or until the supernatant is no longer cloudy.
E: Keep the tubes against the magnet, carefully remove the supernatant and discard.
F: With the tubes still against the magnet, wash with 1 ml of 70% ethanol, dribbling down the opposite side of the beads. Incubate 30 seconds room temperature. Carefully remove the 70% ethanol
G: Repeat wash using a second 1 ml 70% ethanol making sure to remove any remaining ethanol.
H: Remove the tubes from the magnet and air dry the beads, taking care not to over dry them.
I: Elute DNA as follows: add 100 ul low TE (pH 8.0) to each tube and pipette up and down 10 times. Hold against the magnet for 1 min, remove the DNA in solution to a new tube. Combine eluates—final volume 200 ul.

1F: pfosIll(ΔKan) DNA Concentration

Quantitate pfosIll(ΔKan) DNA concentration using the Qubit Fluorometer (Invitrogen) with Quant-it dsDNA BR assay (Invitrogen) according to the manufacturer's instructions. Typically 750-1000 ug of plasmid is recovered. The plasmid is aliquoted and kept at 4° C. for near term use or at −20° C. for long term storage.

1 G: pfosIll(ΔKan) DNA Concentration

To confirm adequate removal of background DNA digest 200 ng of Plasmid Safe DNase treated pfosIll(ΔKan) and 200 ng of non-Plasmid Safe DNase treated pfosIll(ΔKan) with AatII and Eco72I. Use the appropriate buffers and incubate at 37° C. for 1 hr. Run digests and 2 log markers (NEB) on an 0.7% agarose gel in 1×TAE for 4 hrs at 70V and stain using 15 ul Sybr Green I in 150 mls water for 45 min. View gel using a gel imaging system.

Step 2: pfosIll(ΔKan) Arm Preparation

Preparation of pfosIll(ΔKan) vector arms is depicted in FIG. 21.

### Materials

### Lab Instruments

Dark Reader Transilluminator (Clare Chemical Research)

Qubit Fluorometer (Invitrogen, Q32857)

### Store at 4° C.

AmPure XP beads (Beckman Coulter (Agencourt), A63 881)

Ultra Pure Phenol-Chloroform-Isoamylalcohol 25:24:1 (Amresco, K169)

Chloroform-Isoamylalcohol 24:1 (American Bioanalytical, AB0234500500)

NEB 2-Log DNA Ladder (0.1-10.0 kb) (NEB, N3200L)

pFosill(ΔKan) (plasmid safe DNase treated)

### Store at −20° C.

Eco72I (10 U/ul) (Fermentas, ER0361)

AatII (10 U/ul) (Fermentas, ER0992)

Alkaline Phosphatase, Calf Intestinal (10 U/ul) (NEB, M0290S)

10×NEB Buffer 3 (NEB, B70035)

70% Ethanol

Glycogen (Roche, 10901393001)

Sybr Green I (Invitrogen, S-7563)

### Store at RT:

0.5M EDTA

5M NaCl

100% Ethanol

Quant-iT dsDNA BR assay kit (_{0.2-100ng}) (Invitrogen, Q32850)

Amicon Ultra, 0.5 ml 100K column (Millipore, UFC5100967)

MaXtract High Density 1.5 ml (Qiagen, 129046)

Qubit Assay Tubes 0.5 ml (Invitrogen, Q32856)

1×TAE

2A: AatII/Eco72I pfosIll(ΔKan) Digest

Digest 50 ug pfosIll(ΔKan) with AatII and Eco72I

| | ul |
|---|---|
| 10X Tango Buffer | 50 |
| pFosill(ΔKan) (50 ug) | x |
| Water | y |
| AatII (10 U/ul) | 20 |
| Eco72I (10 U/ul) | 20 |
| Final Volume | 500 |

Pipette up and down to mix and digest at 37° C. for 1 hr, then heat inactivate at 65° C. for 20 min. To confirm complete digestion of pfosIll(ΔKan) run 200 ng uncut pfosIll(ΔKan), 200 ng (2 ul) of the digested pfosIll(ΔKan) and NEB 2-Log DNA Ladder on a 0.7% agarose gel in 1×TAE. Run gel at 70V for 4 hrs and stain the gel using 15 ul Sybr Green I in 150 mls water for 45 min and view using an appropriate gel imaging system.

2B: Purification of Digested pfosIll(ΔKan)

Clean up the digested pfosIll(ΔKan) plasmid using Agencourt AMPure XP beads (Beckman Coulter) as follows:
A: Split the DNA into two 1.5 ml Eppendorf tubes.
B: Shake the AMPure XP bottle to resuspend any magnetic particles that may have settled. Add 1.8× vol (450 ul) of AMPure XP beads to each tube.
C: Pipette up and down 10 times and incubate for 5 minutes at room temperature,
D: Hold against a magnet for at least 2 minutes or until the supernatant is no longer cloudy.
E: Keep the tubes against the magnet, carefully remove the supernatant and discard.
F: With the tubes still against the magnet, wash with 1 ml of 70% ethanol, dribbling down the opposite side of the beads. Incubate 30 seconds room temperature. Carefully remove the 70% ethanol.
G: Repeat wash using a second 1 ml 70% ethanol making sure to remove any remaining ethanol.
H: Remove the tube from the magnet and air dry the beads, taking care not to over dry them.
I: Elute DNA as follows: add 100 ul low TE (pH 8.0) to each tube and pipette up and down 10 times. Hold against the magnet for 1 min, remove the DNA in solution to a new tube. Combine eluates—final volume 200 ul.

2C: Dephosphorylate the Digested pfosIll(ΔKan)

The digested pfosIll(ΔKan) was dephosphorylated as follows.

| | ul |
|---|---|
| DNA (50 ug) | 200 |
| Water | 22.5 |
| 10X NEB 3 | 25 |
| CIP | 2.5 |
| Final Volume | 250 |

Incubate at 37° C. for 1 hr. Add 1 ul of CIP and incubate at 55° C. 1 hr.

2D: Phenol Chloroform Extraction of the pfosIll(ΔKan) Arms

Add an equal volume of Phenol Chloroform Isoamylalcohol (25:24:1) to the CIP treated DNA, vortex and transfer to a 1.5 ml MaXtract High Density tube (Qiagen). Spin at 14,500 rpm for 5 min and remove the aqueous layer and pipette into a fresh Eppendorf tube. Add 1 volume of chloroform/Isoamylalcohol (24:1), vortex and transfer to a fresh 1.5 ml MaXtract High Density tube and centrifuge at 14,500 rpm for 5 min. Remove the aqueous layer and pipette DNA into a new tube. Measure the volume and add:
1 ul glycogen,
1/20th volume 5M NaCl
2.1× volume 100% ethanol
   Incubate at −20° C. for one hour, or overnight, spin for 20 min at 13,000 rpm at 4° C. Remove the supernatant and wash the pellet with 0.5 ml prechilled 70% ethanol and spin for 5 min at 13,000 rpm at 4° C. Remove the ethanol and repeat. Dry the DNA pellet and resuspend in 30 ul low TE buffer.

2E: DNA Concentration of pFosill(ΔKan) Arms

Quantitate pfosIll(ΔKan) arms using the Qubit Fluorometer (Invitrogen) with Quant-it dsDNA BR assay (Invitrogen) according to the manufacturer's instructions.

Typically recover ˜40% of AatII/Eco72I DNA (from 50 ug digested pfosIll(ΔKan) recover ˜20 ug arms, ˜600-700 ng/ul). Adjust the concentration of the arms to 500 ng/ul with low TE buffer.

### Example VI

### Part B: Genomic Insert DNA Preparation

Genomic DNA for making fosIlls was prepared by shearing and end repairing the DNA, size selecting using pulse field gel electrophoresis and purifying the DNA using a (3-Agarase based method.

### Preparation of 40 kb Inserts

Notes:

Use wide bore tips to pipette genomic DNA
Wide-Orifice LTS 250 ul pipette tips (Rainin, RT-L250WS)
Wide-Orifice LTS 1000 ul pipette tips (Rainin, RT-L1000WS)

Never vortex the genomic DNA

Avoid exposing DNA to UV light

Materials for Insert Preparation

Lab Instruments
Gene Machine HydroShear
Dark Reader Transilluminator (Clare Chemical Research)
Pulse Field Gel Electrophoresis (Bio-Rad CHEF-DRIII)
Qubit Fluorometer (Invitrogen, Q32857)

Wash Chemicals for HydroShear
Water
0.2M HCl
0.2N NaOH

Stored at −20° C.
β-Agarase I 1 U/ul (NEB, M0392L)
10× β-Agarase I Buffer (NEB, B0392S)
Glycogen (Roche, 10 901 393 001)
70% Ethanol
10× T4 DNA ligase buffer (NEB, B0202S)
10 mM dNTP mix (NEB, N0447S)
Sybr Green I (Invitrogen, S-7563)

### Stored at 4° C.

Ultra Pure Phenol-Chloroform-Isoamylalcohol 25:24:1 (Amresco, K169)
Chloroform-Isoamylalcohol 24:1 (American Bioanalytical, AB0234500500)
DNA Size standards for CHEF pulse field electrophoresis
   5 kb ladder (Bio-Rad, 170-3624)
   8-48 kb (Bio-Rad, 170-3707)

Stored at room temperature
0.006″ shearing assembly for Hydroshear (Bird Precision, RB-82206)
Agarose (IBI scientific, IB70042)
SeaPlaque GTG Agarose (Lonza, 50110)
EB Buffer (Qiagen)
0.5M EDTA
100% Ethanol
low TE (pH 8.0, 10 mM Tris, 0.1 mM EDTA)
5M NaCl
3M Sodium Acetate (pH5.5)
Quant-iT dsDNA HS (0.2-1000 ng) assay kit (Invitrogen, Q32851)
Quant-iT dsDNA BR (2-1000 ng) assay kit (Invitrogen, Q32850)
Wide-Orifice LTS 250 ul pipette tips (Rainin, RT-L250WS)
Wide-Orifice LTS 1000 ul pipette tips (Rainin, RT-L1000WS)
Amicon Ultra, 0.5 ml 100K column (Millipore, UFC5100967)
MaXtract High Density 1.5 ml (Qiagen, 129046)
Qubit Assay Tubes 0.5 ml (Invitrogen, Q32856)
14 ml Falcon Tubes (Falcon, 352059)
5× loading dye (Bio-Rad, 161-0767)
0.5×TBE
Gel Carrier Sheets, 8.5″×11″ UV Transparent (The Gel Company, 6408-100)

Step 1: Shearing Genomic DNA

High molecular weight DNA is sheared to maximize the amount of DNA in the 35-50 kb size range. Some genomic DNA will already be in the correct size range and will not require shearing Shear 30 ug genomic DNA as follows:
Prepare 2×15 ug DNA made up to 125 ul in low TE buffer (pH 8.0)
Shear the DNA using the following parameters,
Assembly: 0.006″
Volume (ul): 125
Cycle #: 60
Speed code: 15
   The Hydroshear is cleaned before and after use according to the manufacturer's instructions.

The sample is loaded, sheared and ejected out of the Hydroshear according to the manufacturer's instructions.

Step 2: End Repair of Genomic DNA

The sheared genomic DNA is end repaired as follows (for each 15 ug sheared DNA):

| | ul |
|---|---|
| 15 ug sheared DNA | 125 |
| 10X Ligase buffer (NEB) | 17.5 |
| 10 mM dNTP's (NEB) | 4.4 |
| T4 DNA polymerase (NEB) | 5 |
| T4 PNK (NEB) | 5 |
| Klenow DNA polymerase (NEB) | 1 |
| Water | 17.1 |
| Final volume | 175 |

Incubate at 20° C. for 30 min, add 17.5 ul EDTA followed by heat inactivation at 70° C. for 10 min Place onto ice.

Step 3: DNA Size Selection

3A: Set Up and Run the Pulse Field Gel Apparatus

Cast a 1% SeaPlaque GTG Agarose (Lonza, 50110) gel in 0.5×TBE into the CHEF-DRIII gel mold according to the manufacturer's instructions. Set up the comb for the gel as follows. Tape 6 wells of a 30 well comb (Bio-Rad, 170-3628) per 7.5 ug of sheared DNA. Marker wells are separated from genomic DNA by at least 1 well. Position the gel in the CHEF-DRIII gel box and add 2.1 L of 0.5×TBE. Set up the CHEF DR III System as follows:

| | Initial | Final | Run | Volts/ | Included |
|---|---|---|---|---|---|
| Block | SW Time | SW Time | Time | cm | Angle |
| 1 | 1.2 | 6 | 19 | 6 | 120 |

Pre-nm the CHEF-DRIII system to cool to the buffer and gel to 14° C. Genomic DNA and DNA ladders were loaded as follows using wide bore tips:

Genomic DNA:
Add 35 ul of 6× loading dye to each 7.5 ug sample (175 ul).
   5 ul (179 ng) of this is loaded into 1 well of the gel (and stained) as a reference for assessing the size of the sheared DNA.
   7.5 ug (205 ul) of genomic DNA is loaded per 6 wells of the pulse field gel.

DNA ladders
300 ng CHEF 8-48 kb (Bio-Rad, heated to 65° C. for 5 min)
300 ng CHEF 5 kb ladder (Bio-Rad)
   Once the gel is loaded, run for 5 min without the pump then turn on the pump at a speed of 35 for 10 minutes then increase to 70. Run the gel overnight at 14° C. (19 hours total).

3B: Excise DNA from the Pulse Field Gel

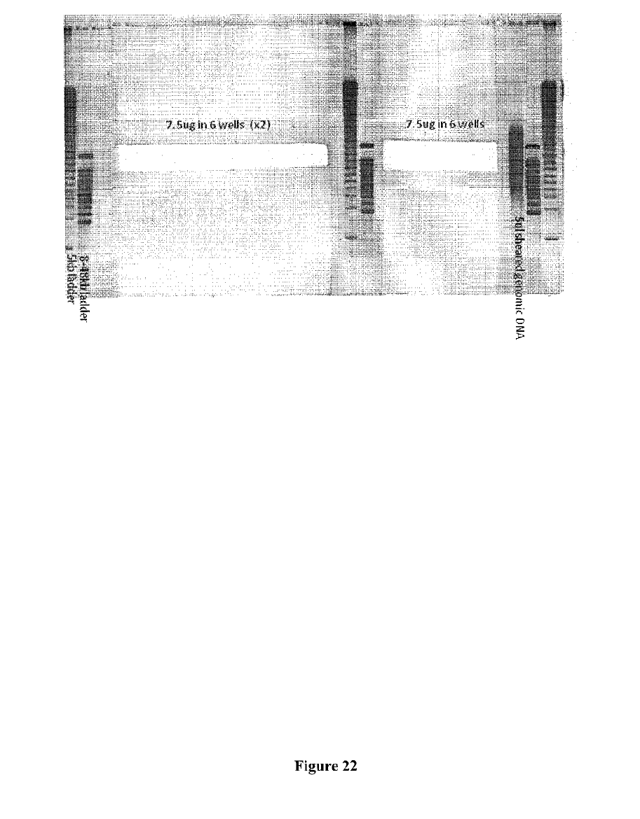

Remove the gel from the CHEF-DRIII gel apparatus. Cut the marker lanes from the sample lanes and stain for 45 minutes using 10 ul Sybr green diluted in 100 mls water. Reconstruct the gel on Gel Carrier Sheets and view on the Dark Reader Transilluminator. Excise the genomic DNA between 33 kb and 48 kb using the CHEF 8-48 kb markers as a reference. Transfer each gel slice to a 14 ml Falcon tube (one per 7.5 ug gel slice). Store at 4° C. until processed. As a backup, excise a gel slice between 48 kb and 55 kb and save this at 4° C. until the size selection of the 33 kb and 48 kb gel slice is confirmed. A pulse field gel with 33 kb to 48 kb and 48 kb and 55 kb gel slices was viewed and stored using an appropriate imaging system. See, FIG. 22.

3C: Purify DNA from the Excised Gel Slice

Size selected DNA is recovered using a β-Agarase based method to digest the agarose. DNA is cleaned up using phenol/chloroform and is then ethanol precipitated. β-Agarase: Weigh the excised agarose gel slices and equilibrate twice using two gel slice volumes (assume 1 g of agarose is equivalent to 1 ml) of 1× β-Agarase buffer plus 40 mM NaCl for 30 min on ice. The buffer is removed. The agarose is melted at 70° C. for 10 min in a water bath. Make sure all the agarose is melted by gently flicking the tube transfer quickly to 42° C. water bath for 5 min. Add 1/100^{th} volume of β-Agarase (e.g. for 2 g agarose add 20 ul β-Agarase). The gel is incubated at 42° C. for 2 hours then a second 1/100^{th} volume of β-Agarase is added followed by incubation for another two hours. (NOTE: do not to allow the agarose to solidify once it has melted as the β-Agarase will not work. It is important to transfer the agarose quickly between the 70° C. and 42° C. water baths and not to leave the tubes containing the melted agarose on the bench).

Each sample is split into two 1.5 ml Eppendorf tubes and incubated at 70° C. for 10 min. The tubes were placed in an ice bath for 5 min and centrifuged at 10,000 rpm for 20 minutes at 4° C. to pellet any insoluble oligosaccharides. Any “pellet” will be gelatinous. Remove the supernatant being careful to avoid the gelatinous pellet. The final volume of the size selected DNA is usually around 2 mls for 7.5 ug of DNA. Store the size selected DNA at 4° C.

### DNA Clean Up & Precipitation

Amicon Ultra, 0.5 ml 100K columns are used to reduce the volumes of the size selected DNA from ˜2 mls to ˜350 ul. Use a wide bore pipette to transfer ˜500 ul of the DNA to a column and spin at 2000 ref for 7 min. Remove the flow through and repeat with more β-Agarase treated DNA until the total volume is reduced to ˜350 ul. Pipette the sample up and down slowly rinsing the sides of the filter using a wide bore pipette, turn the column upside down into a clean collection tube and spin at 1000 rcf for 2 min

Measure the volume of concentrated DNA. Add an equal volume of Phenol Chloroform Isoamylalcohol (25:24:1), mix by inversion and transfer to a 1.5 ml MaXtract High Density tube (Qiagen). Spin at 14,500 rpm for 5 min and remove the aqueous layer and pipette into a second 1.5 ml MaXtract High Density tube. Add 1 volume of chloroform/Isoamylalcohol (24:1), mix by inverting and spin at 14,500 rpm for 5 min. Remove the aqueous layer and pipette DNA into a new tube. Measure the volume and add:
1 ul glycogen,
1/10^{th} vol 3M NaAc pH5.5
2.5× vol 100% ethanol
   Incubate at −20° C. for at least one hour, or overnight, then spin for 20 min at 13,000 rpm at 4° C. Remove the supernatant and wash the pellet with 500 ul pre chilled 70% ethanol and spin for 5 min at 13,000 rpm at 4° C. Remove the ethanol and repeat. Air dry the DNA pellet and resuspend in 12 ul low TE buffer for at least 2 hrs on ice or overnight at 4° C.

Step 4: DNA Concentration

The DNA concentration of the purified DNA was measured using the Quant-iT dsDNA HS kit on the Qubit Fluorometer, according to the manufacturer's instructions. Typically recover 1500-2000 ng from 15 ug starting DNA.

Step 5: Confirm Size Selection

Cast a 1% Agarose (IBI scientific, IB70042) gel in 0.5×TBE using the CHEF-DRIII gel mold according to the manufacturer's instructions. Used the 30 well comb (Bio-Rad, 170-3628). Position the gel in the CHEF-DRIII gel box and add 2.1 L of 0.5×TBE. Set up the CHEF DR III System as follows:

| | Initial | Final | Run | Volts/ | Included |
|---|---|---|---|---|---|
| Block | SW Time | SW Time | Time | cm | Angle |
| 1 | 1 | 5 | 14 | 6 | 120 |

Pre-run the CHEF-DRIII system to cool to the buffer and gel to 14° C. 100 ng of size selected DNA, 300 ng CHEF 8-48 kb ladder (pre-heated to 65° C.) and 300 ng CHEF 5 kb ladders were loaded onto the pulse field gel using wide bore tips. Once the gel is loaded, run for 5 min without the pump then turn on the pump at a speed of 35 for 10 minutes then increase to 70. Run the gel overnight at 14° C. (14 hours total). Remove the gel from the CHEF-DRIII gel apparatus and stain using 10 ul Sybr Green I diluted in 100 mls water for 45 minutes. Visualize the gel using a gel imaging system to confirm size selected DNA is of the appropriate size.

### Example VII

### Part C: Fosmid Preparation

The next step is to make Fosmid libraries. Insert genomic DNA is ligated to the pfosIll(ΔKan) aims. This ligation is then packaged using phage lambda packaging extract and transformed into E. coli from which 40 kb DNA can be isolated.

### Material List:

Materials for Fosmid Prep

Stored at −80° C.
Lambda-Competent cells, E. coli GC10T1 (equivalent to E. coli DH10B-T1)
   (Grown 10 mM MgSO₄ and 0.2% Maltose)
Components from EpiFOS™ Fosmid Library Production Kit (Epicentre, FOS0901)
   MaxPlax™ Lambda Packaging Extracts

Stored at −20° C.
Components from EpiFOS™ Fosmid Library Production Kit (Epicentre, FOS0901)
   Fosmid Control DNA (100 ng/ul)
   pEpiFOS™-5 Fosmid Vector (500 ng/ul)
T4 Ligase 2000 U/ul (NEB M0202M)
10× T4 DNA ligase buffer (NEB, B0202S)

Stored at 4° C.
Size selected genomic DNA
LB agar plates (15 ug/ml chloramphenicol)
pfosIll(ΔKan) arm prep

Stored at room temperature
SM buffer with 0.01% Gelatin
Wide-Orifice LTS 250 ul pipette tips (Rainin, RT-L250WS)
Wide-Orifice LTS 1000 ul pipette tips (Rainin, RT-L1000WS)
70 ul DMSO (Sigma, D2650)
10 mM Magnesium Sulfate
LB media
2XYT media
QIAfilter Plasmid Purification Mega Kit (Qiagen, 12281)

Step 1: Ligation

Size selected genomic DNA was ligated to pfosIll(ΔKan) arms. The following controls were also set up:

Epicentre control arms and inserts (from Epicentre Packaging Kit), as a positive control for ligation and packaging.

pfosIll(ΔKan) anus ligated (with no insert DNA). This is set up the first time using a pfosIll(ΔKan) arms prep, to ensure there are no self ligations (which may cause a background).

pfosIll(ΔKan) arms ligated to the Epicentre control inserts to ensure the arms work well.

Typical ligations set up are shown in the table below.

| | | | | | | | 10× | |
|---|---|---|---|---|---|---|---|---|
| | Arms | Arms | | Insert | Insert | Water | Ligation | Ligase |
| Arms | (ng) | (ul) | Insert | (ng) | (ul) | (ul) | buffer (ul) | (2,000 U/ul) (ul) |
| Epicentre Control | 500 | 1 | Epicentre | 250 | 2.5 | 4.5 | 1 | 1 |
| (500 ng/ul) | | | Control (100 ng/ | | | | | |
| | | | ul) | | | | | |
| pFosill(ΔKan) | 500 | 1 | none | — | 0 | 7 | 1 | 1 |
| (500 ng/ul) | | | | | | | | |
| pFosill(ΔKan) | 500 | 1 | Epicentre | 250 | 2.5 | 4.5 | 1 | 1 |
| (500 ng/ul) | | | Control (100 ng/ | | | | | |
| | | | ul) | | | | | |
| pFosill(ΔKan) | 500 | 1 | Insert DNA | 250 | x | y | 1 | 1 |
| (500 ng/ul) | | | (×ng/ul) | | | | | |

Incubate at 25° C. overnight. Transfer the reaction to 70° C. for 10 min.

Step 2: Packaging Using Phage Lambda Extract

Each ligation was packaged as follows, essentially following the manufacturer's instructions but with some modifications.
1. Set the water bath to 30° C.
2. Cool the ligation reactions on ice and briefly spin using the microcentrifuge.
3. Split each 10 μl ligation into two 5 ul aliquots.
4. Remove 1 tube of the MaxPlax Lambda Packaging Extract from the −80° C. freezer, for each 2×5 ul ligation aliquots, and keep on dry ice.
5. Thaw quickly each packaging tube as needed by holding the tube between your fingers
6. Immediately after thawing, add 25 ul extract to each 5 ul ligation aliquot and mix by tapping.
7. Centrifuge briefly and incubate at 30° C. for 90 min.
8. Remove a 1 tube of the MaxPlax Lambda Packaging Extract from the −80° C. freezer, for each 2×5 ul ligation aliquots, and keep on dry ice.
9. Thaw the packaging extract quickly and add a second 25 ul of MaxPlax Lambda Packaging Extract to each 5 ul ligation/packaging mix and incubate for an additional 90 min at 30° C.
10. Add 940 ul Phage Dilution Buffer (SM buffer with 0.01% Gelatin) and mix gently.
11. Add 70 ul DMSO (Sigma) and mix gently by inversion. The packaged ligations can be stored short term at 4° C. or long term at −80° C.

Step 3: Transformation of E. coli

After phage lambda packaging, the 40 kb DNA is transferred into E. coli where the DNA undergoes in vivo circularization.
1. Remove lambda-competent GC10T1 cells from the −80° C. freezer and transfer to ice. To each 50 ul cell aliquot, add 200 ul 10 mM Magnesium Sulfate and mix by tapping.
2. Pipette 25 ul diluted cells into an Eppendorf tubes and keep on ice. Add 2.5 ul of the packaged ligations, mix by inversion and incubate for 30 min at 37° C. As a negative control, add 2.5 ul SM buffer to 25 ul diluted cells.
3. Add 780 ul of pre warmed LB media, and incubate at 37° C. for 45 min, mixing by inversion every 15 min.
4. Plate out cells onto prewarmed LB agar plates with 15 ug/ml chloramphenicol and incubate overnight at 37° C.
5. Count the colonies formed and calculate for the total packaging extract. The Epicentre positive control typically gives 3-5 million colonies. 250 ng of size selected genomic DNA typically gives 100,000-500,000 colonies. In our experience the quality of starting DNA dramatically affects the success of Fosmid production.

Step 4: Isolation of 40 kb DNA

Once the packaging reactions have been assessed to have the required number of colonies, a large scale transformation can be carried out using the remaining ˜1 ml packaged ligation. A large scale plasmid prep can then be used to isolate the 40 kb circular DNA.

Large Scale Transformation

Often, multiple packaging reactions are needed to obtain the required number of 40 kb inserts. For isolation of 40 kb DNA, up to 3 transformations are combined and grown as a single plasmid prep. The example below combines 3 packaging reactions for a single large scale 40 kb plasmid prep.
1. For each packaging reaction remove 1 ml lambda-competent cells from the −80° C. freezer and transfer to ice. Add 9 ml 10 mM Magnesium Sulphate.
2. Remove three ˜1 ml tubes of packaged ligation reactions (H-DMSO) from −80° C., place on ice and thaw quickly between fingers when ready to add to lambda-competent cells.
3. Add 1 ml packaged reaction to 10 ml diluted lambda-competent cells and incubate at room temperature for 20 min.
4. Add 40 mls LB media prewarmed to 37° C. and shake at 250 rpm for 45 min at 37° C.
5. Plate out 20 ul and 40 ul onto LB agar plates with 15 ug/ml chloramphenicol from the 51 ml culture and grow these overnight at 37° C.
6. Combine the three 51 ml cultures into a 2 L flask and add 600 ml 2XYT media supplemented with 15 ug/ml chloramphenicol.
7. Shake at 250 rpm overnight at 30° C. and grow to an OD₆₀₀ of ˜1-1.5.
8. Make two glycerol stocks from the culture by mixing 500 ul cells with 500 ul 50% glycerol. Transfer to two sterile cryotubes and store at −80° C.
9. Count colonies on the 20 ul and 40 ul LB agar plates to estimate the number of colonies from the packaging reactions.

Large Scale Plasmid Prep

Isolate 40 kb plasmid DNA using a Qiagen QIAfilter Plasmid Mega Purification kit according to the manufacturer's instructions. Briefly,
1. Harvest the bacterial cells by centrifugation at 6000×g for 15 min at 4° C. The cells can be stored at −20° C. or processed to make the 40 kb plasmid prep.
2. Screw the QIAfilter Mega-Giga Cartridge onto a 45 mm-neck glass bottle and connect it to a vacuum source.
3. Resuspend the bacterial pellet in 50 ml of Buffer P1.
4. Add 50 ml of Buffer P2, mix thoroughly by vigorously inverting 4-6 times, and incubate at room temperature for 5 min.
5. Add 50 ml 1 chilled Buffer P3 and mix thoroughly by vigorously inverting 4-6 times. Mix well until white, fluffy material has formed and the lysate is no longer viscous. Proceed directly to step 6. Do not incubate on ice.
6. Pour the lysate into the QIAfilter Mega-Giga Cartridge and incubate at room temperature for 10 min.
7. Switch on the vacuum source. After all liquid has been pulled through, switch off the vacuum source. Leave the QIAfilter Cartridge attached.
8. Add 50 ml Buffer FWB2 to the QIAfilter Cartridge and gently stir the precipitate using a sterile spatula. Switch on the vacuum source until the liquid has been pulled through completely.
9. Equilibrate a QIAGEN-tip 2500 by applying 35 ml Buffer QBT, and allow the column to empty by gravity flow.
10. Apply the filtered lysate from step 10 onto the QIAGEN-tip and allow it to enter the resin by gravity flow
11. Wash the QIAGEN-tip with a total of 200 ml Buffer QC
12. Elute DNA with 29 ml Buffer QF.
13. Precipitate DNA by adding 20.3 ml room-temperature isopropanol to the eluted DNA. Mix and centrifuge immediately at >15,000×g for 30 min at 4° C. Carefully decant the supernatant.
14. Wash DNA pellet with 7 ml of room-temperature 70% ethanol, and centrifuge at ≧15,000×g for 10 min. Carefully decant the supernatant without disturbing the pellet.
15. Air-dry the pellet for 5 min, and resuspend the DNA in 1 ml low TE (pH8.0). Leave overnight to resuspend.

DNA Concentration

The DNA concentration of the 40 kb DNA was measured using the Quant-iT dsDNA HS kit on the Qubit Fluorometer, according to the manufacturer's instructions. Typical DNA concentrations are 20-50 ng/ul. The plasmids are aliquoted and stored at 4° C. for near term use and at −80° C. for long term storage.

### Example VIII

### Part D: fosIlls—Illumina Library Prep

This part of the protocol outlines the steps needed to make an Illumina library from 40 kb fosmid DNA.

Materials
Lab Instruments
   Dark Reader Transilluminator (Clare Chemical Research)
   Qubit Fluorometer (Invitrogen, Q32857)
   GeneAmp PCR system 9700 (96 and 384 well) (Applied Biosystems)
   Pippen Prep (Sage Science)
Lab Supplies
Stored at −20° C.
   Nb.BbvCI (10 U/ul) (NEB, R0631S)
   DNA polymerase I (10 U/ul) (NEB, M209S)
   Invitrogen S1 nuclease (Invitrogen, 18001-016)
   10× S1 reaction buffer (Invitrogen, Y02292)
   S1 Dilution buffer (Invitrogen, Y02294)
   3M NaCl Invitrogen (Invitrogen, Y02293)
   T4 DNA polymerase (3 U/ul) (NEB, M0203L)
   T4 PNK (10 U/ul) (NEB, M0201L)
   Klenow DNA polymerase (5 U/ul) (NEB, M0210S)
   PE1.0/PE2.0 primers (25 uM)
   2× Phusion HF (Finnzymes, F-531)
   70% Ethanol
   T4 Ligase 2000 U/ul (NEB, M0202M)
   10× T4 DNA ligase buffer with 10 mM ATP (NEB, B0202S)
   10 mM dNTP mix (NEB, N0447S)
   Sybr Green I (Invitrogen, S-7563)
Stored at 4° C.
   AmPure XP beads (Beckman Coulter (Agencourt), A63881)
   2-Log DNA Ladder (0.1-10.0 kb) (NEB, N3200L)
Stored at room temperature
   Agarose (IBI scientific, IB70042)
   0.5M EDTA
   100% Ethanol
   5× loading dye (Bio-Rad, 161-0767)
   0.5×TBE
   low TE pH 8.0 (10 mM Tris, 0.1 mM EDTA)
   Quant-iT dsDNA HS assay kit (_{0.2-100ng}) (Invitrogen, Q32851)
   Qubit Assay Tubes 0.5 ml (Invitrogen, Q32856)
   MinElute Gel Extraction Kit (Qiagen, 28604)
   Eppendorf DNA LoBind Tube, 1.5 ml (Eppendorf, 022431021)
   1.5% Pippen Prep Cassette (Sage Science)
   QIAquick PCR Purification kit (Qiagen, 28104)

Step 1: Nick 40 kb Plasmid DNA using Nb.BbvCI

10 ug of the 40 kb Fosill plasmid was nicked as follows:

| Component | Volume (ul) |
|---|---|
| 40 kb Fosill plasmid (10 ug) | x |
| 10X Buffer NEB 2 | 45 |
| 100X BSA (NEB) | 4.5 |
| Water | y |
| Nb.BbvCI (10 U/ul) (NEB) | 5 |
| Final volume | 450 |

Pipette up and down to mix thoroughly. Digest at 37° C. for 1 hr. Clean up nicked DNA using Agencourt AMPure XP beads (Beckman Coulter) as follows:
1. Shake the AMPure XP bottle to resuspend any magnetic particles that may have settled. Add 1.8× vol (810 ul) of AMPure XP beads to the nicked DNA.
2. Pipette up and down 10 times.
3. Split into two 1.5 ml Eppendorf tubes and incubate for 5 minutes at room temperature.
4. Hold against a magnet for at least 2 minutes or until the supernatant is no longer cloudy.
5. Keep the tube against the magnet, carefully remove the supernatant and discard.
6. With the tube still against the magnet, wash with 1 ml of 70% ethanol, dribbling down the opposite side of the beads. Incubate 30 seconds room temperature. Carefully remove the 70% ethanol.
7. Repeat wash using a second 1 ml 70% ethanol making sure to remove any remaining ethanol.
8. Remove the tube from the magnet and air dry the beads, taking care not to over dry them.
9. Elute nicked DNA as follows: add 100 ul low TE (pH 8.0) to each tube and pipette up and down 10 times. Hold against the magnet for 1 min, remove the DNA in solution to a new tube. Combine eluates—final volume 200 ul.
Quantitate DNA using the Qubit Fluorometer (Invitrogen) with Quant-it dsDNA HS assay (Invitrogen) according to the manufacturer's instructions. Typically recover ˜4-6 ug nicked DNA.

Step 2: Nick Translation

Nick translation of Nb.BbvCI nicked 40 kb fosIll plasmid DNA and non-nicked Fosill plasmid DNA

Use LoBind DNA tubes for all subsequent steps.

| Sample | NON-NICKED | NICKED |
|---|---|---|
| 40 kb DNA (ng) | 800 | — |
| 40 kb DNA (ng) | — | 800 |
| Vol DNA (ul) | a | x |
| 10x NEB2 | 20 | 20 |
| 2.5 mM dNTP's (NEB) | 20 | 20 |
| Water | b | y |
| ul before DNA polymerase I | 195.00 | 195.00 |

Mix well by pipetting up and down, spin, incubate
on ice for 5 min before adding DNA polymerase I

| | | |
|---|---|---|
| DNA polymerase I (10 U/ul) (NEB) | 5 | 5 |
| Final Volume | 200 | 200 |

Incubate for 45 min on ice, for non-nicked and nicked fosIll plasmid NOTE: the length of the nick translation can vary depending on DNA polymerase I batch. 45 min should give ˜500-700 bp PCR product. Stop reaction by adding 20 ul 0.5M EDTA to each 200 ul nick translation reaction, Clean up nicked translated DNA using Agencourt AMPure XP beads (Beckman Coulter) as follows:
1. Shake the AMPure XP bottle to resuspend any magnetic particles that may have settled. Add 1.8× vol (360 ul) of AMPure XP beads to the nicked DNA.
2. Pipette up and down 10 times.
3. Incubate for 5 minutes at room temperature.
4. Hold against a magnet for at least 2 minutes or until the supernatant is no longer cloudy.
5. Keep the tube against the magnet, carefully remove the supernatant and discard.
6. With the tube still against the magnet, wash with 1 ml of 70% ethanol, dribbling down the opposite side of the beads. Incubate 30 sec room temperature. Carefully remove the 70% ethanol.
7. Repeat wash using a second 1 ml 70% ethanol making sure to remove any remaining ethanol.
8. Remove the tube from the magnet and air dry the beads, taking care not to over dry them.
9. Elute the nick translated DNA as follows: add 39 ul low TE (pH 8.0) to each tube and pipette up and down 10 times. Hold against the magnet for 1 min, remove the DNA in solution to a new tube.

Step 3: Nuclease S1 Digestion

The 40 kb plasmid DNA has been nicked using Nb.BbvCI. The nicks have moved out of the vector and into the ends of the insert DNA by the nick translation reaction. These nicks are now digested using nuclease S1 (Invitrogen) to release the vector with the ends of the 40 kb DNA from the majority of the insert DNA.

| Sample | NON-NICKED | NICKED |
|---|---|---|
| Nick translated DNA (ul) | 39 | 39 |
| 10x S1 buffer | 5 | 5 |
| 3M NaCl | 5 | 5 |
| Invitrogen 31 nuclease (200 U) | 1 | 1 |
| H2O | 0 | 0 |
| Final Volume | 50 | 50 |

S1 nuclease: mix 1 ul S1 nuclease and 4 ul of dilution buffer to give 200 U/ul. Incubate 37° C. for 15 min. Stop by adding 10 ul 0.5M EDTA. Clean up nuclease S1 digested DNA using Agencourt AMPure XP beads (Beckman Coulter) as follows:
1. Shake the AMPure XP bottle to resuspend any magnetic particles that may have settled. Add 1.8× vol (108 ul) of AMPure XP beads to the nicked DNA.
2. Pipette up and down 10 times.
3. Incubate for 5 minutes at room temperature.
4. Hold against a magnet for at least 2 minutes or until the supernatant is no longer cloudy.
5. Keep the tube against the magnet, carefully remove the supernatant and discard.
6. With the tube still against the magnet, wash with 1 ml of 70% ethanol, dribbling down the opposite side of the beads. Incubate 30 sec room temperature. Carefully remove the 70% ethanol.
7. Repeat wash using a second 1 ml 70% ethanol making sure to remove any remaining ethanol.
8. Remove the tube from the magnet and air dry the beads, taking care not to over dry them.
9. Elute the nuclease S1 digested DNA as follows: add 73 ul low TE (pH 8.0) to each tube and pipette up and down 10 times. Hold against the magnet for 1 min, remove the DNA in solution to a new tube.

Step 4: End Repair

End repair the ends of S1 nuclease digested DNA as follows

| Sample | NON-NICKED | NICKED |
|---|---|---|
| Nuclease S1 digested DNA (ul) | 73 | 73 |
| 10x Ligase buffer (NEB) | 10 | 10 |
| 2.5 mM dNTP (NEB) | 10 | 10 |
| H2O | 0 | 0 |
| T4 DNA polymerase (NEB) | 3 | 3 |
| T4 PNK (NEB) | 3 | 3 |
| Klenow (NEB) | 1 | 1 |
| Final Volume | 100 | 100 |

20° C. for 30 min, add 20 ul 0.5M EDTA. Clean up the end repaired DNA using Agencourt AMPure XP beads (Beckman Coulter) as follows:
1. Shake the AMPure XP bottle to resuspend any magnetic particles that may have settled. Add 1.0× vol (100 ul) of AMPure XP beads to the nicked DNA.
2. Pipette up and down 10 times.
3. Incubate for 5 minutes at room temperature.
4. Hold against a magnet for at least 2 minutes or until the supernatant is no longer cloudy.
5. Keep the tube against the magnet, carefully remove the supernatant and discard.
6. With the tube still against the magnet, wash with 1 ml of 70% ethanol, dribbling down the opposite side of the beads. Incubate 30 sec room temperature. Carefully remove the 70% ethanol
7. Repeat wash using a second 1 ml 70% ethanol making sure to remove any remaining ethanol.
8. Remove the tube from the magnet and air dry the beads, taking care not to over dry them.
9. Elute the end repaired DNA as follows: add 100 ul low TE (pH 8.0) to each tube and pipette up and down 10 times. Hold against the magnet for 1 min, remove the DNA in solution to a new tube.

Step 5: DNA Concentration

DNA concentration of DNA purified from Spri Beads measured using dsDNA HS Quant-iT Qubit assay kit according to the manufacturer's instructions. Typically have 100-150 ng of DNA.

Step 6: Circularization

The entire DNA goes into the circularization.

| Sample | NON-NICKED | NICKED |
|---|---|---|
| volume (ul) | 100.0 | 100.0 |
| 10x T4 ligase buffer (NEB) | 50 | 50 |
| Water | 348.0 | 348.0 |
| NEB ligase (2000 U/ul) | 2 | 2 |
| Final volume | 500 | 500 |

500 ul overnight ligation at 16° C. Clean up circularized DNA using a Qiagen PCR clean up column according to the manufacturer's instructions. Elute using 50 ul low TE.

Step 7: Trial PCR

Test the number of PCR cycles needed for library amplification using a range of PCR cycles

| SAMPLE | NON-NICKED | NICKED | control |
|---|---|---|---|
| DNA (ul) | 2.0 | 4.0 | 0 |
| PE1.0/PE2.0 (25 uM) | 1.0 | 1.0 | 1.0 |
| 2XPhusion (NEB) | 25.0 | 25.0 | 25.0 |
| dH2O | 22.0 | 20.0 | 24.0 |
| final volume | 50.0 | 50.0 | 50 |

Set up PCR reactions and divide each sample into one well of 4×384 well PCR plates. Samples were amplified using a GeneAmp PCR system 9700 (Applied Biosystems) as follows.

| | | | | |
|---|---|---|---|---|
| 98° C. | 30 | secs | | |
| 98° C. | 10 | sec | | |
| 65° C. | 30 | sec | {close oversize bracket} | CYCLE 15/18/21/24 |
| 72° C. | 30 | sec | | |
| 72° C. | 7 | mins | | |

| | |
|---|---|
| 4° C. | indefinite |

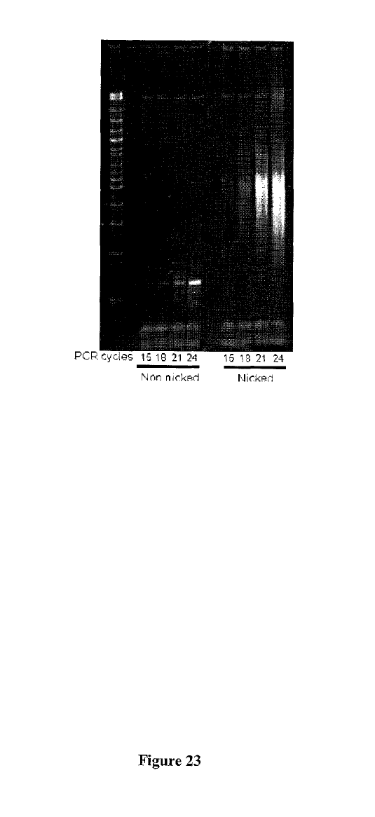

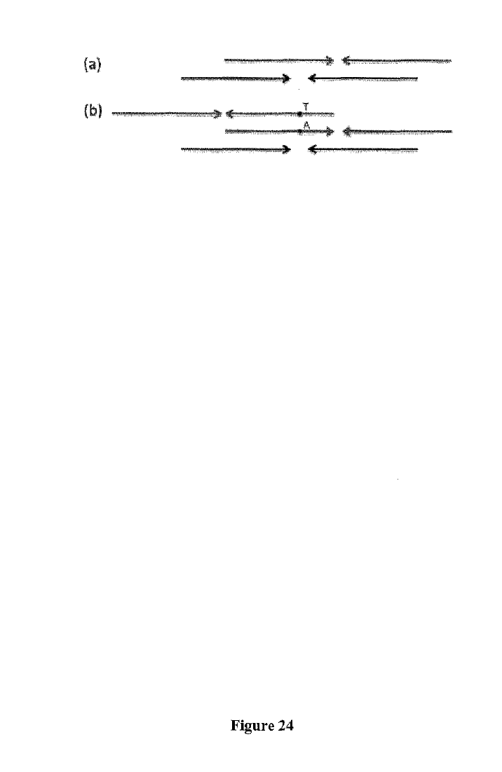

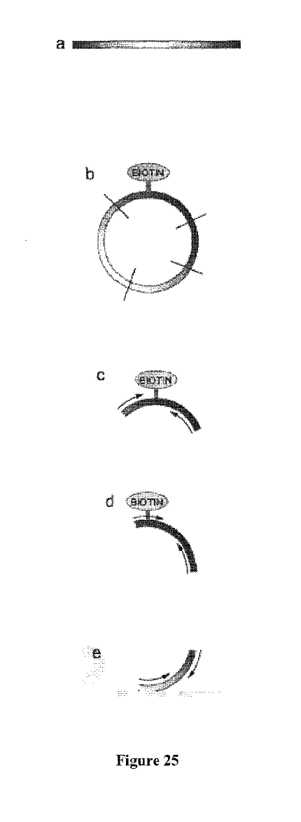

2 ul of 5× loading dye were added to each well and the samples were run on a Criterion 5% precast 1×TBE polyacrylamide gel (Bio-Rad, 345-0049) using the Criterion Cell Gel running system (Bio-Rad, 165-6001) at 120V. The gel was stained using SYBR Green I (10 ul in 100 mls water) for 10 min and viewed on a gel imager. See FIG. 23. The optimal PCR cycles for large scale Fosill library amplification were determined from this gel. In general, a minimal PCR cycle number that gives product is chosen. In this case, 18 PCR cycles were selected. The size of the PCR product depends on the amount of DNA polymerase I added as well as the length of time of the nick translation reaction.

Step 8: Fosill Library Amplification

Large scale PCR of the remaining 46 ul circularized DNA. Use appropriate cycle number as determined from the trial PCR

| SAMPLE | NON-NICKED | NICKED | control |
|---|---|---|---|
| library vol | 48.0 | 48.0 | 0.0 |
| PE1.0/PE2.0 (25 uM) | 4 | 4 | 1 |
| 2XPhusion | 300.0 | 300.0 | 25.0 |
| dH2O | 248.0 | 248.0 | 24.0 |
| final volume | 600.0 | 600.0 | 50.0 |

Each sample was divided into 12 wells of a 96 well PCR plate and was amplified using a GeneAmp PCR system 9700 (Applied Biosystems) as follows.

| | | | |
|---|---|---|---|
| 98° C. | | 3 | mins |
| 98° C. | | 120 | sec |
| 65° C. | {close oversize bracket} | 30 | sec |
| 72° C. | | 30 | sec |
| 72° C. | | 7 | mins |

| | |
|---|---|
| 4° C. | indefinite |

Pool the 12 wells for each sample into one tube. Rinse each set of 12 wells using 10 ul low TE (pH 8.0) and add to the pooled PCR product. Remove 1/200^{th} (3 ul) to check on a gel after size selection of the PCR product. Clean up amplified library using Agencourt AMPure XP beads (Beckman Coulter) as follows:
1. Shake the AMPure XP bottle to resuspend any magnetic particles that may have settled. Add 1.8× vol (900 ul) of AMPure XP beads to the nicked DNA.
2. Pipette up and down 10 times and split into 4 tubes.
3. Incubate for 5 minutes at room temperature.
4. Hold against a magnet for at least 2 minutes or until the supernatant is no longer cloudy.
5. Keep the tubes against the magnet, carefully remove the supernatant and discard.
6. With the tubes still against the magnet, wash with 1 ml of 70% ethanol, dribbling down the opposite side of the beads. Incubate 30 sec room temperature. Carefully remove the 70% ethanol.
7. Remove the tubes from the magnet and air dry the beads, taking care not to over dry them (a couple of minutes).
8. Elute nicked DNA as follows: add 30 ul low TE (pH 8.0) to the first tube. Pipette up and down 10 times and then add this to the next tube. Pipette up and down 10 times and repeat with the remaining 2 tubes. Hold against the magnet for 1 min, remove the DNA in solution to a new tube.

Step 9: Fosill Library Size Selection

Size selection of a 550-800 bp PCR product using the Pippen Prep as follows:
1. Set up a 1.5% Gel Cassette in the Pippen Prep apparatus according to the manufacturer's instructions.
2. Use the following size cut off settings to get the desired 550-8000 bp PCR product size selection.
   a. Lower size cut off: 550 bp
   b. Larger size cut off: 900 bp
   Using these settings we should get back approx 550-800 bp.
3. The 30 ul of PCR product was combined with 10 ul of Pippen prep loading dye and loaded onto the 1.5% Pippen Prep Cassette.
4. The Pippen Prep was run according to the manufacturer's instructions.
5. Remove the size selected PCR product (40 ul) from the cassette.
6. Clean up using Spri beads to remove Ethidium Bromide prior to submitting the library for Illumina sequencing.
   a. Shake the AMPure XP bottle to resuspend any magnetic particles that may have settled. Add 1.8× vol (72 ul) of AMPure XP beads to the nicked DNA.
   b. Pipette up and down 10 times.
   c. Incubate for 5 minutes at room temperature.
   d. Hold against a magnet for at least 2 minutes or until the supernatant is no longer cloudy.
   e. Keep the tubes against the magnet, carefully remove the supernatant and discard.
   f. With the tubes still against the magnet, wash with 1 ml of 70% ethanol, dribbling down the opposite side of the beads. Incubate 30 sec room temperature. Carefully remove the 70% ethanol.
   g. Remove the tubes from the magnet and air dry the beads, taking care not to over dry them (a couple of minutes).
   h. Elute nicked DNA as follows: add 22 ul low TE (pH 8.0). Pipette up and down 10 times and hold against the magnet for 1 min, remove the DNA in solution to a new tube.

Step 10: Fosill Library DNA Concentration

Measured DNA cone using Qubit Quant-iT HS dsDNA assay kit according to the manufacturer's instructions. Typically library concentrations are 0.5-5ng/ul.

Step 11: Confirm Library Size Selection

Check the aliquot of the large scale PCR (removed in Section 7: Fosill Library Amplification) and the aliquot of the gel recovered DNA (removed in Section 8: Fosill Library Size Selection) on a polyacrylamide gel. Both aliquots were made to 8 ul with low TE and had 2 W 5× loading dye added prior to running on a Criterion 5% precast 1×TBE polyacrylamide gel (Bio-Rad, 345-0049) at 120V. The gel was stained using Sybr Green I (10 W in 100 mls water) for 10 min and the gel was viewed on a gel imager,

Step 12: Submit Library for Illumina Sequencing

### Example VII

### pEpiFos-5 Vector Preparation

The example illustrates fosmid library construction using the pEpiFos-5 vector, phage packaging, and transfection techniques. After transfection, the fosmid-containing bacteria were plated on LB agar plates containing 25 mg/ml chloramphenicol and 5% sucrose. To achieve the desired 3-4 million clones, bacteria were plated on 80 plates to a target density of 50,000 colonies per 500 cm² plate. After 18 hour growth, the bacteria were scraped and collected. fosmids were purified by Plasmid Mega Kit (Qiagen) per manufacturer's specifications. The quantity of purified fosmids was determined via Pico Green quantification.

### Example VIII

### fosmid Hydroshearing

This example describes one embodiment of a method for a total of 100 μg of purified fosmid DNA to be sheared to the range of 8-10 kb via Hydroshear (Disposable Shearing Device). Initially, mouse fosmid samples (˜15 ug in 200 uL) were sheared at speed code 11 for 30 cycles using a 0.0020″ orifice assembly. Further experiments yielded a more robust protocol that utilizes 10 ug in 200 uL sheared with speed code 4 for 20 cycles using a 0.0035″ orifice assembly.

A preliminary list of materials and methods include, but not limited to:

### Disposable Shearing Device

Syringe, BD disp 1 ml Luer Lock (VWR BD-309628)
Assembly, Shear. Device Orifice, 0.0020″ (Bird Precision 82206)
Needle, blunt 18G 1.5″ (McMasterCarr 75165A762)
Tubing High Purity PFA, 0.062 In ID×⅛ IN Black (Idex HS 1640)
PEEK ferrule and SS lock ring (Kinesis Inc 008FK32)
M6 fitting Nut Click-N-Seal ⅛″ (Kinesis Inc 008NC32-CS6R)

### Female Leur to M6 Male (Idex HS P-686)

Genomic DNA (20 μg in 200 μl) was loaded into the disposable syringe via the blunt ended needle. The needle was removed and an assembly consisting of the M6 fitting Nut Click-N-Seal, the 0.0020″ orifice assembly, the female leur to M6 male, to the tubing (sealed by the PEEK ferrule and SS lock ring) was attached to the syringe. The entire shearing assembly, with syringe, was inserted into the Disposable Shearing Device. The device was run at Speed 11 for 30 cycles. When finished, the sample was unloaded and run and a portion was run on an 0.6% agarose gel for 2 hours to determine if the material was in the correct size range.

### Example IX

### Agarose Size Selection & End Polishing

Sheared DNA prepared in accordance with Example VIII was concentrated by Qiaquick columns (Qiagen) and post-shearing yield was determined via Pico Green quantification and was typically 75 μg. The DNA was size selected for precisely 8-10 kb by electrophoresis on a 23×14 cm sized 1% low melt agarose—TAE gel. The entire 75 μg sample was spread across 20 wells along side a 2-log DNA ladder (New England Biolabs) and run in a gel box circulating buffer cooled to 11° C. for 16 hours at 35 volts. The gel was stained with SYBR Safe (Invitrogen) for 30 minutes and a band of 8-10 kb was excised from each lane. DNA was extracted from the gel slices with Qiaquick columns according to manufacturer's protocol. Purified fragments were end polished and phosphorylated in a standard T4 DNA Polymerase and T4 Polynucleotide Kinase reaction, followed by enzymatic reaction clean up with Ampure SPRI XP beads (Beckman Genomics).

### Example X

### Re-Circularization and Exonuclease Treatment

The size distribution and quantity of blunted DNA was determined by a DNA 12,000 chip on a Bioanalyzer 2100 (Agilent). The typical yield at this step was 6-8 μg of DNA. A large scale circularization reaction was next performed to re-circularize the fragments.

Briefly, for a 6 μg sample, a 3 ml ligation reaction was set up containing 100 ul of T3 DNA Ligase (3000 U/ul, Enzymatics) in 1× Rapid Ligation Buffer. After incubation for 16 hours at 16° C., the ligation was split across four 1.5 ml tubes and each was cleaned up with 0.8 volumes of Ampure SPRI XP beads, eluting each in 30 ul of 10 mM Tris-HCl (pH 8.5). The four eluates were pooled for a final volume of 120 ul. The circularized DNA was exonuclease treated to remove uncircularized fragments with 15 units of ATP Dependent Plasmid Safe DNAse (Epicentre Bio) in the presence of 1 mM ATP and 1× reaction buffer in a final volume of 150 ul. After incubation for 40 minutes at 37° C., the reaction was quenched by the addition of 4 ul 0.5 M EDTA and was immediately cleaned up with 0.8 volumes of Ampure SPRI XP eluting in 30 ul of 10 mM Tris-HCl (pH 8.5).

### Example XI

### ShARC PCR Selection for Fosmid Ends

ShaRc fosmid fragments recircularized in accordance with Example X containing both fosmid ends were selected for by PCR with primers specific for the pEpiFos-5 vector sequences immediately adjacent to the flanking genomic insert. These primers were tailed with part of the Illumina sequencing paired end adapter (underlined bases) to remove the need for downstream adapter ligation:

ShaRc Tailed Forward:
5′ - ACACTCTTTCCCTACACGACGCTCTTCCGATCTG-
TACCCGGGGATCCCAC - 3′
ShaRc Tailed Reverse:
5′ - CTCGGCATTCCTGCTGAACCGCTCTTCCGATCTT-
CGACTCTAGAGGATCCCAC - 3′

The circularized ShaRe fosmid fragments were evenly split across three 50 μl PCR reactions each containing 1 unit of Phusion Hot Start High Fidelity DNA Polymerase (Finnzymes), 0.2 mM dNTPs, 0.125 mM of each ShaRc primer, and 1× Phusion HF buffer. The reaction was cycled at 98° C. for 30 seconds, 20 cycles of 98° C. for 10 seconds, 65° C. for 30 seconds, and 72° C. for 30 seconds, followed by a final extension at 72° C. for 5 minutes. The three PCR reactions were pooled after cycling and cleaned up with 0.8 volumes of Ampure SPRI XP eluting in 30 μl of 10 mM Tris-HCl (pH 8.5). PCR products were then analyzed on a DNA 12,000 chip on a Bioanalyzer 2100 (Agilent).

### Example XII

### Final Size Selection and Enrichment with Illumina Paired End Primers

To allow for proper cluster amplification downstream in accordance with Example XIII, the size distribution of ShaRc fosmid fragment amplicons were refined by an additional 1% agarose gel purification step excising a band between 500-1000 bp.

ShaRe fosmid fragment amplicons were extracted with Qiaquick columns and eluted in 30 μl of Buffer EB. The entire 30 μl volume of size-selected product was then PCR enriched with Illumina paired end enrichment primers according to the standard Illumina protocol with Phusion High Fidelity Mastermix (Finnzyme) and 10 cycles of amplification, breaking the sample up across 3 reactions each containing 10 μl of DNA. The final enriched libraries were pooled and cleaned up with 0.8 volumes of Ampure SPRI XP, eluting with in 30 μl of 10 mM Tris-HCl (pH 8.5).

### Example XIII

### Cluster Amplification & Sequencing

ShaRc fosmid fragment libraries were quantified by QPCR (KAPA Biosystems) to facilitate proper sequencing adapter sequence addition.

Quantified ShaRc fosmid fragment library samples were then normalized and denatured according to Illumina standard procedure. Because the ShaRc fosmid fragments contain a monotemplate from the pEpiFos vector at the start of both reads, the libraries were loaded at a lower target density under 350,000 clusters per mm² to avoid problems in generating the base calling matrix. All sequencing was done on Illumina GAIIx instruments with 76 base paired reads.

### Example IVX

### Illumina Sample Prep Modifications

Construction of fosmid fragment libraries followed standard protocols for shearing of genomic DNA, end repair, adapter ligation, and size selection. The enrichment PCR was performed using AccuPrime Taq DNA polymerase High Fidelity (Invitrogen) with the following cycling conditions: initial denaturation for 3 min at 98° C., followed by 10 cycles of denaturation for 80 s at 98° C., annealing and extension for 90 s at 65° C., and a final extension for 10 min at 65° C.

### Example XV

### Generic ShaRc Fosmid Fragment Library Construction Method

fosmid libraries were constructed as follows, using the pEpiFOS-5 fosmid vector (EpiCentre Biotechnologies).

Genomic DNA (˜20 μg) was sheared with a HydroShear device (Disposable Shearing Device) and end-repaired. Size-selected (˜35 to 45 kb) fragments were blunt-end ligated to into the pEpiFOS-5 vector. Primary packaged fosmid libraries were transfected into E. coli DH10B, spread at high density on LB plates containing 25 mg/mL chloramphenicol and 5% sucrose, and incubated at 37° C. for 18 h to select for the fosmid clones. Colonies were then scraped, collected, and purified via a Plasmid Mega Kit (Qiagen).

fosmids were then sheared by HydroShear to an average size of 9 kb to produce a subset of fragments containing the vector backbone and several hundred bases of the genomic insert on either side (i.e., a genomic flanking insert). The sheared fragments were concentrated with QIAquick columns, agarose gel size selected to 8-10 kb, purified, end-repaired, phosphorylated, and cleaned up using SPRI beads. The size of the remaining fragments was assayed using an Agilent 2100 Bioanalyzer.

Next, large-scale recircularization was carried out with 6 μg of fosmid fragments in a 3-mL reaction containing 100 μL of T3 DNA ligase in a 1× rapid ligation buffer (NEB). The cleaned-up, eluted ligation products (120 μL) were treated with 15 units of DNase (Epicentre) to remove linear DNA. Circularized fragments containing both fosmid ends were selected by PCR using primers derived from the pEpiFOS-5 sequences adjacent to the genomic flanking insert, which were tailed with Illumina paired-end adapter sequences. The PCR product was then gel size selected to 500-1,000 bp, enriched using standard Illumina paired-end primers, and sequenced.

### Example XVI

### fosIll-2 Gel Free Method

A gel free method of making fosmids would simplify and speed up the process of making Fosill libraries. Instead of using pulse filed gel electrophoresis to enable size selection, the packaging of the arms/insert DNA into lambda phage extract can be used as a size selection step.

Size selection of genomic DNA using pulse field gel electrophoresis is the standard way to make Fosmids. The size selected DNA is then ligated to vector “arms”. This step is necessary to reduce the formation of chimeras.

To eliminate the gel purification step, barcoded oligonucleotides with SapI overhangs can be ligated to insert DNA. This results in insert DNA with non-complementary ends.

These can be ligated to pfosIll-2(BlpI/BssHII) vector “arms” and eliminate chimeras forming due to ligation between insert DNA's. Packaging within phage lambda extract then serves as the size selection step as phage lambda heads can accept DNA sized between 38 kb and 52 kb.

1-16. (canceled)

17. A composition comprising a circular first nucleic acid vector sequence comprising a cloning site,
wherein said cloning site is flanked by at least one pair of adapter sequences and by at least one pair of nicking endonuclease sites.

18. (canceled)

19. The composition of claim 17, wherein said cloning site is further flanked by a pair of polymerase chain reaction enrichment primer binding sites.

20-22. (canceled)

23. The composition of claim 17, wherein said said each adapter sequence in the pair of adapter sequences comprises one or more of: a sequencing primer binding site, an enrichment primer binding site, a bridge amplification primer sequence, an emulsion amplification primer sequence, a universal primer sequence, a high-throughput sequencing adapter, a stuffer sequence and a barcoded adapter sequence.

24. The composition of claim 17, wherein said first nucleic acid vector sequence comprises a fosmid vector sequence.

25-27. (canceled)

28. The composition of claim 17, wherein said pair of adapter sequences is selected from a genome specific barcode or a species specific barcode.

29-34. (canceled)

35. The composition of claim 17, wherein said composition further comprises a ShaRc fosmid fragment.

36. A composition comprising a plurality of vectors of claim 17 contained within a plurality of microbial clones, wherein each of said vectors comprises a first nucleic acid sequence comprising a cloning site, wherein said cloning site comprises at least two restriction enzyme recognition site clusters and is flanked by a universal primer sequence pair and a nicking endonuclease site pair, wherein the plurality of vectors further comprise a plurality of inserted second nucleic acid sequences.

37-43. (canceled)

44. The composition of claim 36, wherein said universal primer sequence pair comprises a primer sequence selected from a bridge amplification primer sequence and an emulsion amplification primer sequence.

45. (canceled)

46. The composition of claim 17, wherein said nicking endonuclease site pair comprises a Nb/BbvC1 endonuclease site pair.

47-64. (canceled)

65. A method comprising:
(a) incorporating genomic nucleic acid insert ranging between approximately 10-1000 kb into a cloning site of an F-plasmid-derived vector, wherein said cloning site comprises at least two polylinkers and is flanked by at least one adapter sequence pair and a nicking endonuclease site pair, thereby creating a fosmid;
(b) transfecting said fosmid into a microbe capable of being transfected by said F-plasmid-derived vector, thereby forming a fosmid clone library;
(c) amplifying said fosmid library in vivo by growing said library of transfected microbes in a suitable culture medium,
(d) extracting and purifying said circular fosmids;
(e) cleaving said cloned inserts to create a first portion and a second portion, wherein said first portion is less than said second portion, and said first portion remains attached to said F-plasmid-derived cloning vector, and said second portion is released from said cloning vector;
(f) recircularizing said cloning vector by co-ligating the terminal ends of said first portion; and
(g) amplifying said first portion by inverse polymerase chain reaction using said universal primer pair, thereby creating a plurality of linear amplicons configured for at least one next-generation sequencing platform.

66-69. (canceled)

70. The method of claim 65, wherein said method further comprises nicking said endonuclease site under conditions that allow the nick to move by nick translation of several hundred base pairs into said cloned insert.

71. The method of claim 65, wherein said endonuclease site comprises a Nb/BbvC1 endonuclease site.

72. The method of claim 70, wherein double-strand breaks at said translated nicks are generated by S1 nuclease.

73. The method of claim 65, wherein said fosmid cloning library comprises a fosIll cloning library.

74. The method of claim 65, wherein said adapter sequence comprises one or more of: a sequencing primer binding site, an enrichment primer binding site, a bridge amplification primer sequence, an emulsion amplification primer sequence, a universal primer sequence, a high-throughput sequencing adapter, a stuffer sequence and a barcoded adapter sequence.

75-77. (canceled)

78. A method of claim 65, wherein
(h) said recircularized cloning vectors of step (f) of claim 65 comprise a genomic nucleic acid insert ranging between approximately 2-1000 kb in the cloning site of said vector, wherein said cloning site is flanked by an Illumina adapter sequence pair binding site and a polymerase chain reaction enrichment primer binding site, thereby creating a plurality of clones;
(i) transfecting said plurality of clones into a microbe capable of being transfected by said vector, thereby forming a clone genomic library;
(h) amplifying said genomic library in vivo by growing said library of transfected microbes in a suitable culture medium,
(k) extracting and purifying said amplified clone deoxyribonucleic acid;
(l) hydroshearing said amplified clone deoxyribonucleic acid to create a first portion and a second portion, wherein said first portion comprises said vector flanked by a first genomic nucleic acid sequence and second genomic nucleic acid sequence and said second portion is released from said cloning vector;
(m) re-circularizing said first portion by co-ligating the terminal ends of said first genomic nucleic acid sequence and second genomic nucleic acid sequence, thereby creating a circularized first portion; and
(n) amplifying said circularized first portion with an enrichment primer specific for said PCR enrichment primer binding site, thereby creating a plurality of linear amplicons comprising a mate read pair configured for at least one next-generation sequencing platform.

79-90. (canceled)

91. A method comprising:
(a) sequencing short read genomic inserts from a ShaRc fosmid fragment library, wherein said library comprises said short read genomic inserts and a fosmid plasmid derived cloning vector sequence, with a next-generation sequencing platform; and
(b) assembling said short read genomic inserts into a complete genome using a microprocessor comprising a genome assembly algorithm,
wherein said genome assembly algorithm comprises a step for trimming said fosmid plasmid derived cloning vector sequence from said short read genomic inserts.

92-95. (canceled)

96. The method of claim 91, wherein said ShaRc fosmid fragment library is a pooled ShaRc fosmid fragment library.

97.-112. (canceled)

113. The composition of claim 17, wherein the cloning site comprises a PmII restriction site.

114. The composition of claim 17, wherein the at least one pair of nicking endonuclease sites are Nb/BbvC1 endonuclease sites.
